(19) United States
(12) Patent Application Publication
(10) Pub. No.: US 2019/0083521 A1
(43) Pub. Date: Mar. 21, 2019

(54) COMBINATION THERAPY FOR TREATING CANCER

(71) Applicant: Epizyme, Inc., Cambridge, MA (US)

(72) Inventors: Christine Klaus, Waban, MA (US); Maria Alejandra Raimondi, Jamaica Plain, MA (US); Scott Richard Daigle, Newburyport, MA (US); Roy MacFarlane Pollock, Medford, MA (US)

(21) Appl. No.: 15/512,527

(22) PCT Filed: Aug. 12, 2015
(86) PCT No.: PCT/US2015/044907

§ 371 Date: Mar. 17, 2017

Related U.S. Application Data
(60) Provisional application No. 62/051,890, filed on Sep. 17, 2014.
(60) Provisional application No. 62/088,498, filed on Dec. 5, 2014.
(60) Provisional application No. 62/112,086, filed on Feb. 4, 2015.
(60) Provisional application No. 62/165,169, filed on May 21, 2015.
(60) Provisional application No. 62/203,285, filed on Aug. 10, 2015.

(51) Int. Cl. A61K 31/7076; A61K 31/7068; A61K 31/704; A61K 31/706; A61K 31/519; A61K 45/06; A61P 35/02; A61K 31/136; A61K 31/135; A61K 31/635; A61K 31/5377

(52) U.S. Cl. CPC A61K 31/7076; A61K 31/7068; A61K 31/704; A61K 31/706; A61K 31/519; A61K 31/5377; A61P 35/02; A61K 31/136; A61K 31/135; A61K 31/635; A61K 45/06

(57) ABSTRACT

The disclosure relates to combinations comprising inhibitors of human histone methyltransferase DOT1L and one or more therapeutic agents, particularly anticancer agents, and methods of combination therapy for administering to subjects in need thereof for the treatment of cancer.

### RELATED APPLICATIONS

This application is a U.S. National Phase application, filed under 35 U.S.C. § 371, of International Application No. PCT/US2015/044907, filed Aug. 12, 2015, which claims priority to, and the benefit of U.S. Provisional Application No. 62/051,890, filed Sep. 17, 2014; 62/088,498, filed Dec. 5, 2014; 62/112,086, filed Feb. 4, 2015; 62/165,169, filed May 21, 2015; and 62/203,285, filed Aug. 10, 2015. The entire contents of each of these applications are incorporated herein by reference in their entireties.

### FIELD

This disclosure relates to compositions or combinations comprising inhibitors of human histone methyltransferase DOT1L and one or more other therapeutic agents, particularly anticancer agents, and methods of combination therapy for treating cancer.

### BACKGROUND

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., DOT1L), many of which are associated with specific genetic alterations that can cause human disease.

Disease-associated chromatin-modifying enzymes (e.g., DOT1L) play a role in diseases such as proliferative disorders, metabolic disorders, and blood disorders. Thus, there is a need for the development of compositions or combination therapies that are capable of modulating the activity of DOT1L.

### SUMMARY

In one aspect, this present invention features a combination of a DOT1L inhibitor or pharmaceutically acceptable salts thereof, and one or more therapeutic agents.

In another aspect, this present invention features a combination comprising a compound of Formula (I):

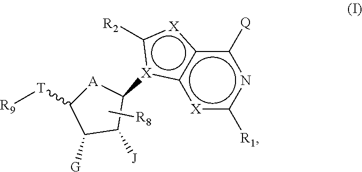

or pharmaceutically acceptable salts thereof, and one or more therapeutic agents, wherein,

T is a linker group of a 6-10 carbon atoms, in which one or more carbon atoms are optionally replaced with a heteroatom and T is optionally substituted;

R₉ comprises a C₆-C₁₀ aryl or 5 to 10-membered heteroaryl optionally substituted with one or more substituents selected from the group consisting of unsubstituted or substituted t-butyl, CF₃, cyclohexyl, C₆-C₁₀ aryl, and 5 to 10-membered heteroaryl;

A is 0 or CH₂;

each of G and J, independently, is H, halo, C(O)OH, C(O)O—C₁-C₆ alkyl or ORₐ, Rₐ being H, C₁-C₆ alkyl, C(O)—C₁-C₆ alkyl, or silyl, wherein C(O)O—C₁-C₆ alkyl, C₁-C₆ alkyl or C(O)—C₁-C₆ alkyl is optionally substituted with one or more substituents selected from the group consisting of halo, cyano hydroxyl, carboxyl, C₁-C₆ alkoxyl, amino, mono-C₁-C₆ alkylamino, di-C₁-C₆ alkylamino, and C₃-C₈ cycloalkyl;

each X independently is N or CRₓ, in which Rₓ is H, halo, hydroxyl, carboxyl, cyano, or R_{S1}, R_{S1} being amino, C₁-C₆ alkoxyl, C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{S1} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;

each of R₁ and R₂, independently is H, halo, hydroxyl, carboxyl, cyano, or R_{S2}, R_{S2} being amino, C₁-C₆ alkoxyl, C₁-C₆ alkyl, C₂-C₆ alkenyl, C₂-C₆ alkynyl, or C₃-C₈ cycloalkyl, and each R_{S2} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;

R₈ is H, halo or R_{S3}, R_{S3} being C₁-C₆ alkyl, C₂-C₆ alkenyl, or C₂-C₆ alkynyl, and R_{S3} being optionally substituted with one or more substituents selected from the group consisting of halo, hydroxyl, carboxyl, cyano amino, C₁-C₆ alkoxyl, mono-C₁-C₆ alkylamino, di-C₁-C₆ alkylamino, and C₃-C₈ cycloalkyl; and

Q is H, NH₂, NHR_{b}, NR_{b}R_{c}, R_{b}, ═O, OH, or OR_{b}, in which each of R_{b} and R_{c} independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, 5 to 10-membered heteroaryl, or -M₁-T₁ in which M₁ is a bond or C₁-C₆ alkyl linker optionally substituted with halo, cyano, hydroxyl or C₁-C₆ alkoxyl and T₁ is C₃-C₈ cycloalkyl, C₆-C₁₀ aryl, 4 to 6-membered heterocycloalkyl, or 5 to 10-membered heteroaryl, or R_{b} and R_{c}, together with the N atom to which they attach, form 4 to 7-membered heterocycloalkyl having 0 or 1 additional heteroatoms to the N atom optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl, and each of R_{b}, R_{c}, and T₁ is optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl.

As used herein, a DOT1L inhibitor is an inhibitor of DOT1L-mediated protein methylation (e.g., an inhibitor of histone methylation). A DOT1L inhibitor may be a small molecule inhibitor of DOT1L.

In some embodiments, the DOT1L inhibitor and the one or more therapeutic agents of the combination of the disclosure are formulated in the same formulation. In other embodiments, the DOT1L inhibitor and the one or more therapeutic agents of the combination of the disclosure are formulated in separate formulations and are administered simultaneously, sequentially or in alternation.

In some embodiments, the combination comprises Compound A2, a DOT1L inhibitor, having the formula:

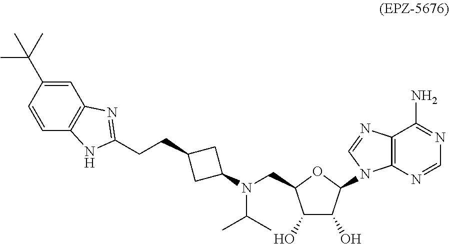 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, polymorph, solvate, or stereoisomer thereof.

In some embodiments, the combination comprises Compound D16, a DOT1L inhibitor, having the formula:

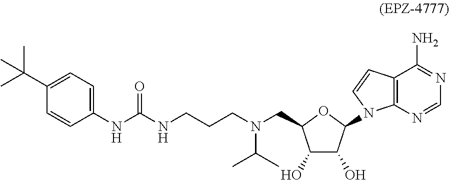 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or a pharmaceutically acceptable salt, polymorph, solvate, or stereoisomer thereof.

“Compound A2” (also known as “EPZ-5676” or pinometostat) and Compound D16 (also known as “EPZ-4777” and “Compound T”) are examples of a compound of Formula (I).

Other DOT1L inhibitors suitable for use according to methods described herein are provided in WO2012/075381, WO2012/075492, WO2012/082436, WO2012/75500, WO2014/026198, WO2014/035140, US2014/0100184, and in J. Med Chem. (2013), 56: p. 8972-8983, the contents of each of which are hereby incorporated by reference in their entireties.

In some embodiments, the one or more therapeutic agents are anti-cancer agents. The one or more therapeutic agents can be selected from Ara-C, Daunorubicin, Azacitidine, Decitabine, Panobinostat, Vidaza, Mitoxantrone, Methotrexate, Mafosfamide, Prednisolone, Vincristine, Lenalidomide, Hydroxyurea, Menin-MLL inhibitor MI-2, JQ1, IBET151, Vorinostat, Quizartinib, Midostaurin, Tranylcypromine, LSD1 inhibitor II, Navitoclax, Velcade, SRT-1720, Furazolidone, Fludarabine, Mercaptopurine, Obatoclax, ABT-199, Trametinib, Clofarabine, Ibrutinib, Palbociclib, AZ20, MK2206, BEZ235, T0070907, Romidepsin, Tipifarnib, Volasertib, Compound E10, 10-Hydroxycamptothecin, ABT-737, Alitretinoin, AT7867, Auranofin, AZD 8055, AZD6244, Baricitinib, BEP800, Bexarotene, BIX01294, Bleomycin Sulfate, BMN 673, BMS 345541, BMS-754807, BX-912, C 646, CAL-101, CAPE, Cerivastatin Sodium, Chlorambucil, Cisplatin, CPI-203, Dabrafenib, GSK-LSD1, Erlotinib Hydrochloride, Etoposide, Everolimus, Fostamatinib disodium, GDC-0941, Go 6976, GSK2656157, IKK-2 Inhibitor VIII, Irinotecan Hydrochloride, JNJ 26854165, KU 0063794, Lapatinib, LB42708, LDN 57444, LEE011, LY2603618, Melphalan, Menadione, Methylprednisolone, Mitomycin C, MK-2206, MLN2238, MS 436, MS-275, NKH 477, NU 7441, Nutlin-3, Olaparib, OTX015, Oxaliplatin, Papaverine Hydrochloride, Parthenolide, PHA-793887, Pomalidomide, Raloxifene Hydrochloride, SB-505124, SCH772984, SGC-CBP30, SMER 3, Sorafenib, SRT1720, TANSHINONE IIA, Temsirolimus, Thiostrepton, Thiotepa, Topotecan Hydrochloride, Tretinoin, Triciribine, UNC 0646, VE-821, XL147, or functional analogs, derivatives, prodrugs, and metabolites thereof. The one or more therapeutic agents can be selected from Ara-C, Daunorubicin, Decitabine, Vidaza, Mitoxantrone, JQ1, IBET151, Panobinostat, Vorinostat, Quizartinib, Midostaurin, Tranylcypromine, LSD1 inhibitor II, Navitoclax, and analogs, derivatives, or combinations thereof. Preferably, the therapeutic agent is Ara-C or Daunorubicin, or an analog or derivative thereof.

In certain embodiments, the one or more therapeutic agents are selected from inhibitors in the RAS-RAF-MEK-ERK pathway, for example, inhibitors that target any one or more of active, inactive, or mutated forms of RAS (small G protein), BRAF (MAPKKK), MEK (MAPKK), and ERK (MAPK).

In certain embodiments, the one or more therapeutic agents are selected from PPAR antagonists, e.g., a PPARγ antagonist such as T0070907 or GW9662.

In one aspect, the disclosure provides a pharmaceutical composition comprising a therapeutically effective amount of any combination described herein and a pharmaceutically acceptable carrier.

In one aspect, the disclosure provides a method of treating or alleviating a symptom of a disease by administering to a subject in need thereof a therapeutically effective amount of a combination described herein. The disease is cancer or a precancerous condition. Alternatively, the disease can be influenced by modulating the methylation status of histones or other proteins. The methylation status is mediated at least in part by the activity of DOT1L.

In one aspect, the disclosure provides a method of treating or alleviating a symptom of cancer by administering to a subject in need thereof a therapeutically effective dose of a compound of Formula (I) (e.g., EPZ-5676 or EPZ-4777) or a pharmaceutically acceptable salt, polymorph, solvate, or stereoisomer thereof and one or more therapeutic agents, where a compound of Formula (I) (e.g., EPZ-5676 or EPZ-4777) or a pharmaceutically acceptable salt, polymorph, solvate, or stereoisomer thereof and the one or more therapeutic agents are administered simultaneously or sequentially. Alternatively, a compound of Formula (I) (e.g., EPZ-5676 or EPZ-4777) or a pharmaceutically acceptable salt, polymorph, solvate, or stereoisomer thereof is administered prior to administration of the one or more therapeutic agents. Alternatively, one or more therapeutic agents are administered/delivered prior to administration of a compound of Formula (I) (e.g., EPZ-5676 or EPZ-4777) or a pharmaceutically acceptable salt, polymorph, solvate, or stereoisomer thereof.

In one aspect, the disclosure provides a method of treating or alleviating a symptom of cancer by administering to a subject in need thereof a therapeutically effective dose of a compound of Formula (I) (e.g., EPZ-5676 or EPZ-4777) or a pharmaceutically acceptable salt, polymorph, solvate, or stereoisomer thereof, prior to administering a therapeutically effective dose of a combination described herein.

In one aspect, the disclosure provides a method of treating or alleviating a symptom of cancer by administering to a subject in need thereof a therapeutically effective dose of one or more therapeutic agents prior to administering a therapeutically effective dose of a combination described herein.

In some embodiments, the combination or composition described herein is administered to the subject in need thereof at a dosage of 0.01 mg/kg per day to about 1000 mg/kg per day.

In some embodiments, the compound of Formula (I) (e.g., EPZ-5676 or EPZ-4777) or a pharmaceutically acceptable salt, polymorph, solvate, or stereoisomer thereof is administered at a dosage of 0.01 mg/kg per day to about 1000 mg/kg per day.

In some embodiments, each of the one or more therapeutic agents is administered at a dosage of 0.01 mg/kg per day to about 1000 mg/kg per day.

In some embodiments, the compound of Formula (I) (e.g., EPZ-5676 or EPZ-4777) or a pharmaceutically acceptable salt, polymorph, solvate, or stereoisomer thereof is administered at a dose of at least 36 mg/m²/day.

In some embodiments, the compound of Formula (I) or a pharmaceutically acceptable salt, polymorph, solvate, or stereoisomer thereof is administered at a dose of at least 45 mg/m²/day.

In some embodiments, the compound of Formula (I) (e.g., EPZ-5676 or EPZ-4777) or a pharmaceutically acceptable salt, polymorph, solvate, or stereoisomer thereof is administered at a dose of at least 54 mg/m²/day.

In some embodiments, the compound of Formula (I) or a pharmaceutically acceptable salt, polymorph, solvate, or stereoisomer thereof is administered at a dose of at least 70 mg/m²/day.

In some embodiments, the compound of Formula (I) (e.g., EPZ-5676 or EPZ-4777) or a pharmaceutically acceptable salt, polymorph, solvate, or stereoisomer thereof is administered at a dose of at least 80 mg/m²/day.

In some embodiments, the compound of Formula (I) or a pharmaceutically acceptable salt, polymorph, solvate, or stereoisomer thereof is administered at a dose of at least 90 mg/m²/day.

In some embodiments, the compound of Formula (I) (e.g., EPZ-5676 or EPZ-4777) or a pharmaceutically acceptable salt, polymorph, solvate, or stereoisomer thereof is administered continuously for at least 7, 14, 21, 28, 35, 42, 47, 56, or 64 days.

In some embodiments, continuous administration comprises administration without a drug holiday.

In some embodiments, the administration results in maturation or differentiation of leukemic blast cells. For example, at least 20% of leukemic blast cells have undergone maturation or differentiation. For example, at least 50% of leukemic blast cells have undergone maturation or differentiation. For example, at least 80% of leukemic blast cells have undergone maturation or differentiation.

In some embodiments, administration results in reduction of H3K79 methyl mark to at least 90%, 80%, 70%, 60%, 50%, 40%, 30%, 20%, 10% or less of untreated control levels.

In some embodiments, administration results in the suppression of H3K79 methyl mark rebound.

In some embodiments, administration results in at least 20%, 30%, 40%, 50%, 60%, 70%, 80%, or 90% of leukemic blast cells undergoing cell death or apoptosis.

In some embodiments, the method of treatment includes resolution of fevers, resolution of cachexia or resolution of leukemia cutis.

In some embodiments, the method of treatment includes restoration of normal haematopoiesis.

In some embodiments, the subject has demonstrated resistance to any one of the components of a combination described herein when administered as a single agent.

In some embodiments, the subject has a mutation in the RAS-RAF-MEK-ERK pathway (e.g., one or more mutations in RAS, one or more mutations RAF, one or more mutations in MEK, and/or one or more mutations in ERK). For example, the subject has a Ras mutation (e.g., H-Ras or HRAS mutation, K-Ras or KRAS mutation, or N-Ras or NRAS mutation).

In some embodiments, the KRAS mutation is at A146. In some embodiments, the KRAS mutation is KRAS A146T. In some embodiments, the KRAS mutation is heterozygous. In some embodiments, the KRAS mutation is heterozygous KRAS A146T or KRAS A146T (het). In some embodiments, the KRAS mutation is at K117. In some embodiments, the KRAS mutation is KRAS K117N. In some embodiments, the KRAS mutation is homozygous. In some embodiments, the KRAS mutation is homozygous KRAS K117N or KRAS K117N (homo).

In some embodiments, the NRAS mutation is at Q61. In some embodiments, the NRAS mutation is NRAS Q61R. In some embodiments, the NRAS mutation is heterozygous. In some embodiments, the NRAS mutation is heterozygous NRAS Q61R or NRAS Q61R (het). In some embodiments, the NRAS mutation is at G12. In some embodiments, the NRAS mutation is NRAS G12D. In some embodiments, the NRAS mutation is homozygous. In some embodiments, the NRAS mutation is heterozygous.

In some embodiments, the subject has an activating mutation in the RAS-RAF-MEK-ERK pathway (e.g., one or more activating mutations in RAS, one or more activating mutations RAF, one or more activating mutations in MEK, and/or one or more activating mutations in ERK).

In some embodiments, the mutation in the RAS-RAF-MEK-ERK pathway results in an upregulation of the RAS-RAF-MEK-ERK pathway.

In some embodiments, the subject is a pediatric patient aged 3 months to 18 years.

In one aspect, the disclosure provides a method of inhibiting cancer cell proliferation by contacting a cancer cell with a combination described herein.

In one aspect, the disclosure provides a method of inhibiting cancer cell proliferation by contacting a cancer cell with a compound of Formula (I) (e.g., EPZ-5676 or EPZ-4777) or a pharmaceutically acceptable salt, polymorph, solvate, or stereoisomer thereof and one or more therapeutic agents, where the compound of Formula (I) (e.g., EPZ-5676 or EPZ-4777) or a pharmaceutically acceptable salt, polymorph, solvate, or stereoisomer thereof and the therapeutic agents are delivered simultaneously or sequentially. Alternatively, a compound of Formula (I) (e.g., EPZ-5676 or EPZ-4777) or a pharmaceutically acceptable salt, polymorph, solvate, or stereoisomer thereof is administered/delivered prior to administration of the therapeutic agents. Alternatively, one or more therapeutic agents are administered/delivered prior to administration of a compound of Formula (I) (e.g., EPZ-5676 or EPZ-4777) or a pharmaceutically acceptable salt, polymorph, solvate, or stereoisomer thereof.

In one aspect, the disclosure provides a method of inhibiting cancer cell proliferation by contacting a cancer cell a therapeutically effective dose of a compound of Formula (I) (e.g., EPZ-5676 or EPZ-4777) or a pharmaceutically acceptable salt, polymorph, solvate, or stereoisomer thereof prior to administering/contacting a therapeutically effective dose of a combination described herein. Alternatively, one or more therapeutic agents are administered/delivered prior to administration of a combination described herein.

The disclosure further provides a method of treating or alleviating a symptom of a disease by administering to a subject in need thereof a therapeutically effective amount of a compound of Formula (I) (e.g., EPZ-5676 or EPZ-4777) or a pharmaceutically acceptable salt, polymorph, solvate, or stereoisomer thereof, where the therapeutically effective amount is an amount sufficient to sensitize the subject to subsequent treatment with a therapeutic agent. The method may further include a step of administering to the sensitized subject a therapeutically effective amount of a therapeutic agent.

The disclosure further provides a method of treating or alleviating a symptom of a disease by administering to a subject in need thereof a therapeutically effective amount of one or more therapeutic agents, where the therapeutically effective amount is an amount sufficient to sensitize the subject to subsequent treatment with a compound of Formula (I) (e.g., EPZ-5676 or EPZ-4777) or a combination that includes one or more therapeutic agents and a compound of Formula (I) (e.g., EPZ-5676 or EPZ-4777) or a pharmaceutically acceptable salt, polymorph, solvate, or stereoisomer thereof. The method may further include a step of administering to the sensitized subject a therapeutically effective amount of a compound of Formula (I) (e.g., EPZ-5676 or EPZ-4777) or a combination that includes one or more therapeutic agents and a compound of Formula (I) (e.g., EPZ-5676 or EPZ-4777) or a pharmaceutically acceptable salt, polymorph, solvate, or stereoisomer thereof.

In certain embodiments, the therapeutic agent is administered at least one, two, three or more hours following the administration of compound of Formula (I) (e.g., EPZ-5676 or EPZ-4777) or a pharmaceutically acceptable salt, polymorph, solvate, or stereoisomer thereof.

In certain embodiments, the therapeutic agent is administered at least one, two, three or more hours prior to the administration of compound of Formula (I) (e.g., EPZ-5676 or EPZ-4777) or a pharmaceutically acceptable salt, polymorph, solvate, or stereoisomer thereof.

In certain embodiments, the therapeutic agent is administered at least one, two, three or more days following the administration of compound of Formula (I) or a pharmaceutically acceptable salt, polymorph, solvate, or stereoisomer thereof.

In certain embodiments, the therapeutic agent is administered at least one, two, three or more days prior to the administration of compound of Formula (I) (e.g., EPZ-5676 or EPZ-4777) or a pharmaceutically acceptable salt, polymorph, solvate, or stereoisomer thereof.

For example, the compound of Formula (I) has the formula

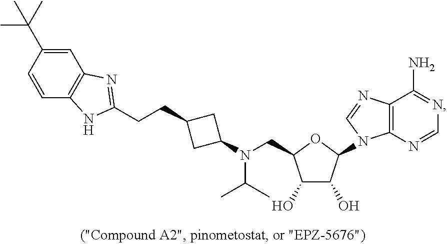 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, polymorph, solvate, or stereoisomer thereof.

For example, the compound of Formula (I) has the formula

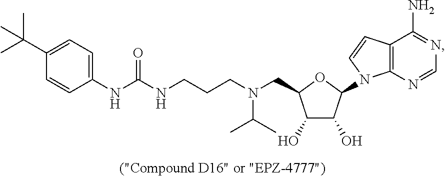 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or a pharmaceutically acceptable salt, polymorph, solvate, or stereoisomer thereof.

In certain embodiments, the sensitization is determined by the methylation status of histones or other proteins.

In certain embodiments, the sensitization is determined by a decreased level of methylation of histones of other proteins, wherein the level is decreased compared to a non-sensitized subject.

In certain embodiments, the sensitization is determined by decreased level of methylation of H3K79.

In certain embodiments, the therapeutically effective amount of the therapeutic agent is lowered due to the sensitizing effect of compound of Formula (I) (e.g., EPZ-5676 or EPZ-4777) or a pharmaceutically acceptable salt, polymorph, solvate, or stereoisomer thereof.

In any methods described herein, the therapeutic agent may be Ara-C or Daunorubicin, or an analog or derivative thereof. Alternatively, the therapeutic agent is a standard of care agent.

In certain embodiments, the therapeutic agent is cytarabine.

In certain embodiments, the therapeutic agent is trametinib.

The subject may have leukemia. The leukemia may be characterized by a chromosomal rearrangement. The chromosomal rearrangement is chimeric fusion of mixed lineage leukemia gene (MLL) or partial tandem duplication of MLL (MLL-PTD).

The subject may have an increased level of HOXA9, Fms-like tyrosine kinase 3 (FLT3), MEIS1, MEIS2, TBP, BCL, and/or DOT1L.

The subject may have a Ras mutation (e.g., H-Ras or HRAS mutation, K-Ras or KRAS mutation, or N-Ras or NRAS mutation).

In some embodiments, the KRAS mutation is at A146. In some embodiments, the KRAS mutation is KRAS A146T. In some embodiments, the KRAS mutation is heterozygous. In some embodiments, the KRAS mutation is heterozygous KRAS A146T or KRAS A146T (het). In some embodiments, the KRAS mutation is at K117. In some embodiments, the KRAS mutation is KRAS K117N. In some embodiments, the KRAS mutation is homozygous. In some embodiments, the KRAS mutation is homozygous KRAS K117N or KRAS K117N (homo).

In some embodiments, the NRAS mutation is at Q61. In some embodiments, the NRAS mutation is NRAS Q61R. In some embodiments, the NRAS mutation is heterozygous. In some embodiments, the NRAS mutation is heterozygous NRAS Q61R or NRAS Q61R (het). In some embodiments, the NRAS mutation is at G12. In some embodiments, the NRAS mutation is NRAS G12D. In some embodiments, the NRAS mutation is homozygous. In some embodiments, the NRAS mutation is heterozygous.

In some embodiments of any methods described herein, the compound of Formula (I) is Compound A2 or Compound D16. In some embodiments, the compound is a pharmaceutically acceptable salt, polymorph, solvate, or stereoisomer of Compound A2 or Compound D16.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Unless specifically stated or obvious from context, as used herein, the terms “a,” “an,” and “the” are understood to be singular or plural. Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Any of the above aspects and embodiments can be combined with any other aspect or embodiment.

Other features and advantages of the disclosure will be apparent from the following detailed description and claims.

### BRIEF DESCRIPTION OF DRAWINGS

FIG. 1 is a diagram showing the overall experimental design and data analysis.

FIGS. 2A-2B are diagrams showing the steps of experimental design. FIG. 2A shows 4-day+3-day (“4+3”) treatment experimental design and FIG. 2B shows 7-day treatment experimental design.

FIG. 3 is diagram showing the experimental design about dosing of the compounds.

FIGS. 4A-4B are graphs showing combination index (CI) values for combinations of Compound A2 and Ara-C. FIG. 4A shows 4+3 treatment and FIG. 4B shows 7-day treatment experiments in MOLM-13 cell line.

FIGS. 5A-5B are graphs showing combination index (CI) values for combinations of Compound A2 and Daunorubicin. FIG. 5A shows 4+3 treatment and FIG. 5B shows 7-day treatment experiments in MOLM-13 cell line.

FIGS. 6A-6B are graphs showing combination index (CI) values for combinations of Compound A2 and hypomethylating agents. FIG. 6A shows combination of Compound A2 and Decitabine and FIG. 6B shows combination of Compound A2 and Vidaza in a 7-day treatment experiment in MOLM-13 cell line.

FIG. 7 is a graph showing combination index (CI) values for combinations of Compound A2 and topoisomerase inhibitor, Mitoxantrone, in MOLM-13 cell line.

FIG. 8 is a graph showing combination index (CI) values for combinations of Compound A2 and Bromodomain inhibitor, IBET-151, in a 7-day treatment experiment in MOLM-13 cell line.

FIGS. 9A-9B are graphs showing combination index (CI) values for combinations of Compound A2 and Ara-C. FIG. 9A shows 4+3 and FIG. 9B shows 7-day treatment experiments in MV4-11 cell line.

FIGS. 10A-10B are graphs showing combination index (CI) values for combinations of Compound A2 and Daunorubicin. FIG. 10A shows 4+3 and FIG. 10B shows 7-day treatment experiments in MV4-11 cell line.

FIG. 11 is a graph showing combination index (CI) values for combinations of Compound A2 and Vidaza in MV4-11 cell line.

FIG. 12 is a graph showing combination index (CI) values for combinations of Compound A2 and topoisomerase inhibitor, Mitoxantrone, in MV4-11 cell line.

FIG. 13 is a graph showing combination index (CI) values for combinations of Compound A2 and HDAC inhibitor, Panobinostat, in MV4-11 cell line.

FIGS. 14A-14B are graphs showing combination index (CI) values for combinations of Compound A2 and IBET-151. FIG. 14A shows 4+3 and FIG. 14B shows 7-day treatment experiments in MV4-11 cell line.

FIGS. 15A-15B are graphs showing combination index (CI) values for combinations of Compound A2 and Tranylcypromine in a 7-day treatment experiment. FIG. 15A shows MOLM-13 cell line and FIG. 15B shows MV4-11 cell line.

FIGS. 16A-16C are graphs showing combination index (CI) values for combinations of Compound A2 and Bcl-2 inhibitor, Navitoclax. FIG. 16A shows a 7-day treatment experiment in MOLM-13 cell line; FIG. 16B shows a 4+3 treatment experiment in MV4-11 cell line; and FIG. 16C shows a 7-day treatment experiment MV4-11 cell line.

FIG. 17 is a graph showing combination index (CI) values for combinations of Compound A2 and FLT inhibitor, Quizartinib, in a 7-day treatment experiment in MV4-11 cell line.

FIGS. 18A-18B are Fa-CI plots showing that Compound A2 and cytarabine act synergistically to induce an antiproliferative effect in the Molm-13 cell line in a pre-treatment model. FIG. 18A shows ten-day continuous dosing of Compound A2 with addition of cytarabine at day 7 showed a range of fractional effects with CI values <1 denoting synergy. FIG. 18B shows that Compound A2 was removed at day 7 prior to the addition of cytarabine showing durable combination benefit.

FIG. 19 shows three treatment models (A, B and C) for the study presented herein.

FIGS. 20A-20D show the data analysis using Chou-Talalay method. Synergy quantification is performed using the Chou-Talalay method for drug combination. An Exemplary combination experiment is shown in FIG. 20A. The Combination Index (CI) equation offers a quantitative definition for additivity (CI=1), synergism (CI<1), and antagonism (CI>1). This equation (shown in FIG. 20B) used Fa values from a constant ratio of drug combination to determine CI values. The resulting plot (Fa-CI) plot (as shown in FIG. 20C) shows the resultant CI values bracketed by 95% confidence intervals. These Fa-CI plots are generated using the Calcusyn software. Statistically significant CI values for synergy are for example those CI value<1 with the confidence interval lines also below 1. FIG. 20D shows an exemplary combination experiment result using this data analysis.

FIGS. 21A-21B are plots demonstrating synergistic and durable response with combination of Compound A2 and AML standard of care drugs in MLL-r leukemia cell lines. FIG. 21A shows that Compound A2 demonstrates synergistic antiproliferative activity in combination with standard of care (SOC) drugs for AML in MLL-rearranged leukemia cell lines MOLM-13 (panels a and b) and MV4-11 (panels c and d). Cells were treated according to the pre-treatment model described in the Methods Section A (no Compound A2 washout). Synergistic anti-proliferative activity of Compound A2 in combination with AML SOC agents was also observed when cells were treated according to the co-treatment model described in the Methods Section B (data not shown). FIG. 21B shows synergistic anti-proliferative activity between Compound A2 and AML SOC agents is maintained in MOLM-13 (panels a and b) and MV4-11 (panels c and d) MLL-rearranged cells following Compound A2 washout prior to the addition of the SOC agent. Cells were treated according to the pre-treatment model described in the Methods Section A (with Compound A2 washout).

FIGS. 22A-22D are plots showing that cotreatment of Compound A2 with standard of care agent Ara-C demonstrates increased fraction of apoptotic cells in a time and dose dependent manner. FIG. 22A shows that Compound A2 as a single agent induces a dose dependent increase in apoptotic cells after 7 days of treatment. FIG. 22B shows that Compound A2 and Ara-C act synergistically to enhance apoptosis in MLL-rearranged MOLM-13 cells. Compound treatments were performed as described in the Methods section under treatment for mechanism of cell death studies. In A and B, data represent mean of percentage of gated cells in each stage of apoptosis. **Day 14 resulted in fewer cell events. Green stacks represent percentages of cells in early stage apoptosis (means+/−S.D., n=3). ****P<0.0001 (ANOVA plus Bonferroni's post-test) Combination of Compound A2 with Ara-C compared with Compound A2 alone, #### P<0.0001 (ANOVA plus Bonferroni's post-test) combination of Compound A2 with Ara-C compared with Ara-C alone. FIG. 22C shows representative apoptosis dot plots of MOLM-13 cells on Day 10. Cells were treated with DMSO (panel a), Compound A2 (panel b), Ara-C (panel d) or the combination of Ara-C and Compound A2 (panel d). FIG. 22D shows a synergistic increase in apoptosis was detected by an increase in the percent of cells in sub-G1 phase of the cell cycle and an increase in the percentage of cells staining positive for annexin-V. Similar results were observed when Compound A2 was combined with Daunorubicin (data not shown).

FIGS. 23A-23B are plots demonstrating that Compound A2 increases expression of differentiation markers as single agent and in combination with Ara-C in the MOLM-13 cells. FIG. 23A shows that Compound A2 and Ara-C as single agents and in combination promote time and concentration dependent up-regulation of the differentiation markers CD11b and CD14 (data not shown) in MLL-rearranged MOLM-13 cells. FIG. 23B shows that IgG was utilized as a control. Cells were harvested at day 10 (panels a, b, and c) or day 14 (panels d, e, and f) for measuring the markers. Cells were treated with Compound A2 (panels a and d), Ara-C (panels b and e) or the combination (panels c and f). Cultures treated as described in the Methods section for mechanism of cell death studies.

FIGS. 24A-24B are plots showing that Compound A2 does not enhance anti-proliferative effect of standard of care drugs in non-MLL rearranged SKM-1 cells. Compound A2 has no single agent activity in non-MLL rearranged cell line SKM-1 and no augmentation of antileukemic activity was observed upon treatment with a combination of standard of care drugs and Compound A2 according to the co-treatment model described in the Methods section. FIG. 24A shows combination of Compound A2 and Ara-C and FIG. 24B shows combination of Compound A2 and Daunorubicin.

FIGS. 25A-25C are plots showing that Compound A2 demonstrates strong synergy with DNMT inhibitor Azacytidine in MLL-rearranged cell lines. Compound A2 and azacytidine synergistically induce an anti-proliferative effect in co-treatment models of MLL-rearranged leukemia. FIG. 25A shows MOLM-13 cell line and FIG. 25B shows MV4-11 cell line. FIG. 25C shows that Azacytidine single agent activity was not potentiated by Compound A2 in the non-rearranged SKM-1 cell line.

FIGS. 26A-26D are treatment schemes for the study presented herein. FIG. 26A shows a pre-treatment model. FIG. 26B shows a co-treatment model. FIG. 26C shows a treatment model for mechanism of action studies. FIG. 26D shows a pre-treatment model for reverse order of addition.

FIGS. 27A-27B are graphs showing combination therapy of Ara-C and Compound A2. Synergy is observed when cells are pretreated with Ara-C followed by cotreatment with Compound A2. Combination benefit is maintained when Ara-C is washed out prior to treatment with compound A2. FIG. 27A shows Ara-C Treatment for 3 Days followed by Compound A2 and Ara-C co-treatment for 7 Days. FIG. 27B shows Ara-C Treatment for 3 Days followed by Compound A2 Treatment for 7 Days (washout Ara-C).

FIGS. 28A-28D are graphs demonstrating that Compound A2 induces a synergistic and durable antiproliferative effect in combination with AML Standard of Care Drugs in MLL-rearranged leukemia cell lines. Cells were treated with Compound A2 continuously. FIG. 28A shows the combination of Compound A2 and Ara-C in MOLM-13 cells. FIG. 28B shows the combination of Compound A2 and Daunorubicin in MoLM-13 cells. FIG. 28C shows the combination of Compound A2 and Ara-C in MV4-11 cells. FIG. 28D shows the combination of Compound A2 and Daunorubicin in MV4-11 cells.

FIGS. 29A-29D are graphs showing that Compound A2 induces a synergistic and durable antiproliferative effect in combination with AML Standard of Care Drugs in MLL-rearranged leukemia cell lines. Compound A2 was washed out. FIG. 29A shows the combination of Compound A2 and Ara-C in MOLM-13 cells. FIG. 29B shows the combination of Compound A2 and Daunorubicin in MoLM-13 cells. FIG. 29C shows the combination of Compound A2 and Ara-C in MV4-11 cells. FIG. 29D shows the combination of Compound A2 and Daunorubicin in MV4-11 cells.

FIGS. 30A-30B are graphs showing that combination benefit is maintained when cells are pretreated with Ara-C prior to cotreatment with Compound A2 and durable upon removal of Ara-C after pretreatment in the MOLM-13 cell line. FIG. 30A shows Ara-C and Compound A2 co-treatment and FIG. 30B shows Ara-C washout before Compound A2 treatment.

FIGS. 31A-31B are graphs showing that Compound A2 (also called EPZ-5676 or 5676 in all the experiments described herein) does not enhance anti-proliferative effect of standard of care drugs in non-MLL rearranged SKM-1 cells. FIGS. 31A shows the combination of Compound A2 and Ara-C and FIG. 31B shows the combination of Compound A2 and Daunorubicin.

FIGS. 32A-32D are graphs showing that Compound A2 increases expression of differentiation markers and apoptosis as single agent and in combination with standard of care drugs in the MOLM-13 cell line. FIG. 32A shows percent change of viable cells, early stage apoptosis, late stage apoptosis and nuclear debris in cells treated with DMSO or different dosage of Compound A2 alone. FIG. 32B show percent change of viable cells, early stage apoptosis, late stage apoptosis and nuclear debris in cells treated with DMSO or different combination of Compound A2 with standard care of drugs. FIG. 32C shows the distribution of cell cycle stages at various time points for MOLM-13 cells treated with DMSO (control), 156 nM Compound A2, 63 nM Ara-C or a combination of Compound A2 and Ara-C. FIG. 32D is a kinetic plot for the sub-G1 cell population.

FIGS. 33A-33D are graphs showing the same results of FIGS. 32A-32D in a different format. FIGS. 33A and 33B show the late and early apoptosis progress curves of cells treated with Compound A2 alone, Ara-C alone, or combination of Compound A2 and Ara-C. Cells in FIG. 33B received a pretreatment. FIGS. 33C and 33D show the cell cycle progress curves of cells treated with Compound A2 alone, Ara-C alone, or combination of Compound A2 and Ara-C. Cells in FIG. 33D received a pretreatment.

FIGS. 34A-34C are panels showing that Compound A2 increase expression of differentiation marker and apoptosis as single agent and in combination with standard of care drugs in the MOLM-13 cell line. FIG. 34A shows marker CD11 b, FIG. 34B shows marker CD14 and FIG. 34C shows control marker IgG. Each small panel in each figure corresponds to a treatment regimen: cells in panel a were treated with Compound A2 alone and harvested at day 10; cells in panel b were treated with Compound A2 alone and harvested at day 14; cells in panel c were treated with Ara-C alone and harvested at day 10; cells in panel d were treated with Ara-C alone and harvested at day 14; cells in panel e were treated with Compound A2 and Ara-C and harvested at day 10; cells in panel f were treated with Compound A2 and Ara-C and harvested at day 14.

FIGS. 35A-35C are graphs showing that Compound A2 demonstrates strong synergy with DNMT inhibitor Azacytidine in MLL-rearranged cell lines and other chromatin modifying agents. FIG. 35A shows MOLM-13 cells. FIG. 35B shows MV4-11 cells. FIG. 35C shows SKM-1 cells.

FIGS. 36A-36B are graphs showing the effects from Compound A2 and Rosiglitazone co-treatment of MOLM-13 cells (FIG. 36A) and Compound A2 and T0070709 co-treatment of MOLM-13 cells (FIG. 36B).

### DETAILED DESCRIPTION

The disclosure is based upon the discovery that DOT1L histone methyltransferase inhibitors and anti-cancer agents can be used in combination to treat tumors and with superior results than those achieved by treating tumors with DOT1L histone methyltransferase inhibitors alone or anti-cancer agents alone.

Accordingly, the disclosure provides a combination of a DOT1L histone methyltransferase inhibitor and one or more therapeutic agents, and methods for their use to treat diseases the course of which can be influenced by modulating the methylation status of histones or other proteins, e.g., cancer. In particular, the disclosure features a composition or combination comprising Formula (I), e.g., Compound A2 or Compound D16, and Ara-C, Azacitidine, or Daunorubicin.

In some embodiments, the present disclosure provides a composition or combination comprising Formula (I), e.g., Compound A2 or Compound D16, and an inhibitor of the RAS-RAF-MEK-ERK pathway. In some embodiments, the inhibitor of the RAS-RAF-MEK-ERK pathway is a MEK inhibitor. In some embodiments, the inhibitor is trametinib.

The disclosure also includes methods for combination therapies comprising DOT1L histone methyltransferase inhibitor and one or more therapeutic agents, such as a compound of Formula (I), e.g., EPZ-5676 or EPZ-4777, and Ara-C, Azacitidine, or Daunorubicin, to treat cancer, e.g., leukemia. Specifically, the methods of the disclosure are useful for treating or inhibiting cancer cell proliferation.

The disclosure further provides uses of any composition or combination described herein in the manufacture of medicament for treating diseases. Such diseases include, for example, cancer, a precancerous condition, or a disease influenced by modulating the methylation status of histones or other proteins.

Any compound (e.g., DOT1L inhibitor) disclosed herein can be used for the compositions or combination therapy of the disclosure. As used herein, a DOT1L inhibitor is an inhibitor of DOT1L-mediated protein methylation (e.g., an inhibitor of histone methylation). In some embodiments, a DOT1L inhibitor is a small molecule inhibitor of DOT1L.

In one aspect, a composition or combination of the disclosure comprises a compound of Formula (I) (e.g., EPZ-5676 or EPZ-4777) or a pharmaceutically acceptable salt, polymorph, solvate, or stereoisomer thereof, and one or more therapeutic agents. The compounds of Formula (I) (e.g., EPZ-5676 or EPZ-4777) are suitable for administration as part of a combination therapy with one or more therapeutic agents or treatment modality, suitable to be administered together, sequentially, or in alternation.

In some embodiments, the DOT1L inhibitor and the one or more therapeutic agents of the combination of the disclosure are formulated in the same formulation. In other embodiments, the DOT1L inhibitor and the one or more therapeutic agents of the combination of the disclosure are formulated in separate formulations and are administered simultaneously, sequentially or in alternation.

The disclosure provides the compounds of Formula (I):

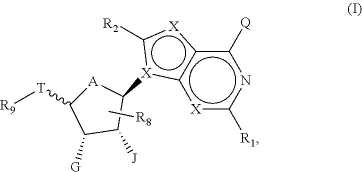

or a pharmaceutically acceptable salt, polymorph, solvate, or stereoisomer thereof, wherein,
T is a linker group of a 6-10 carbon atoms, in which one or more carbon atoms are optionally replaced with a heteroatom and T is optionally substituted;
R₉ comprises a C₆-C₁₀ aryl or 5 to 10-membered heteroaryl optionally substituted with one or more substituents selected from the group consisting of unsubstituted or substituted t-butyl, CF₃, cyclohexyl, C₆-C₁₀ aryl, and 5 to 10-membered heteroaryl;
A is 0 or CH₂;
each of G and J, independently, is H, halo, C(O)OH , C(O)O—C₁-C₆ alkyl or ORₐ, Rₐ being H, C₁-C₆ alkyl, C(O)—C₁-C₆ alkyl, or silyl, wherein C(O)O—C₁-C₆ alkyl, C₁-C₆ alkyl or C(O)—C₁-C₆ alkyl is optionally substituted with one or more substituents selected from the group consisting of halo, cyano hydroxyl, carboxyl, C₁-C₆ alkoxyl, amino, mono-C₁-C₆ alkylamino, di-C₁-C₆ alkylamino, and C₃-C₈ cycloalkyl;
each X independently is N or CRₓ, in which Rₓ is H, halo, hydroxyl, carboxyl, cyano, or R_{S1}, R_{S1} being amino, C₁-C₆ alkoxyl, C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{S1} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
each of R₁ and R₂, independently is H, halo, hydroxyl, carboxyl, cyano, or R_{S2}, R_{S2} being amino, C₁-C₆ alkoxyl, C₁-C₆ alkyl, C₂-C₆ alkenyl, C₂-C₆ alkynyl, or C₃-C₈ cycloalkyl, and each R_{S2} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
R₈ is H, halo or R_{S3}, R_{S3} being C₁-C₆ alkyl, C₂-C₆ alkenyl, or C₂-C₆ alkynyl, and R_{S3} being optionally substituted with one or more substituents selected from the group consisting of halo, hydroxyl, carboxyl, cyano amino, C₁-C₆ alkoxyl, mono-C₁-C₆ alkylamino, di-C₁-C₆ alkylamino, and C₃-C₈ cycloalkyl; and
Q is H, NH₂, NHR_{b}, NR_{b}R_{c}, R_{b}, ═O, OH, or OR_{b}, in which each of R_{b} and R_{c} independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, 5 to 10-membered heteroaryl, or -M₁-T₁ in which M₁ is a bond or C₁-C₆ alkyl linker optionally substituted with halo, cyano, hydroxyl or C₁-C₆ alkoxyl and T₁ is C₃-C₈ cycloalkyl, C₆-C₁₀ aryl, 4 to 6-membered heterocycloalkyl, or 5 to 10-membered heteroaryl, or R_{b} and R_{c}, together with the N atom to which they attach, form 4 to 7-membered heterocycloalkyl having 0 or 1 additional heteroatoms to the N atom optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl, and each of R_{b}, R_{c}, and T₁ is optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl.

The disclosure relates to a composition comprising one or more therapeutic agents and (i) a compound selected from Compound A2 and Compound D16; (ii) a salt, polymorph, solvate, or stereoisomer of a compound selected from Compound A2 and Compound D16; (iii) an N-oxide of a compound selected from Compound A2 and Compound D16; or (iv) a salt, polymorph, solvate, or stereoisomer of an N-oxide of a compound selected from Compound A2 and Compound D16. For example, the disclosure relates to a composition comprising one or more therapeutic agents and a compound selected from Compound A2 and Compound D16.

In one embodiment, a composition comprises one or more therapeutic agents and the DOT1L inhibitor Compound A2 (also called “Cpd A2”, or pinometostat, or “5676”, or “EPZ-5676”) having the formula:

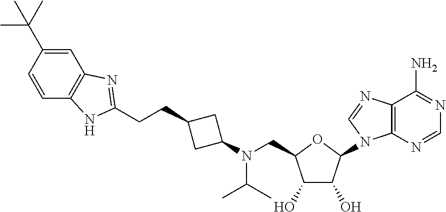 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, polymorph, solvate, or stereoisomer thereof.

In one embodiment, a composition comprises one or more therapeutic agents and the DOT1L inhibitor Compound D16 (also called “Compound T” or “EPZ-4777”) having the formula:

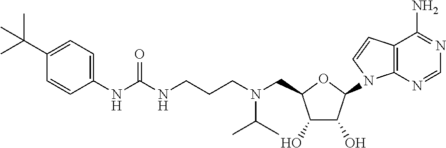 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or a pharmaceutically acceptable salt, polymorph, solvate, or stereoisomer thereof.

Other DOT1L inhibitors suitable for use according to methods described herein are provided in WO2012/075381, WO2012/075492, WO2012/082436, WO2012/75500, WO2014/026198, WO2014/035140, US2014/0100184, and in J. Med Chem. (2013), 56: p. 8972-8983, the contents of each of which are hereby incorporated by reference in their entireties.

The disclosure also relates to a pharmaceutical composition of a therapeutically effective amount of any combination described herein and a pharmaceutically acceptable carrier.

The disclosure also relates to a pharmaceutical composition of a therapeutically effective amount of one or more therapeutic agents and a compound of any of the Formulae disclosed herein and a pharmaceutically acceptable carrier.

The disclosure also relates to a pharmaceutical composition of a therapeutically effective amount of one or more therapeutic agents and a salt of a compound of any of the Formulae disclosed herein and a pharmaceutically acceptable carrier.

The disclosure also relates to a pharmaceutical composition of a therapeutically effective amount of one or more therapeutic agents and a hydrate of a compound of any of the Formulae disclosed herein and a pharmaceutically acceptable carrier.

The disclosure also relates to a pharmaceutical composition of a therapeutically effective amount of one or more therapeutic agents and a polymorph of a compound of any of the Formulae disclosed herein and a pharmaceutically acceptable carrier.

The disclosure also relates to a pharmaceutical composition of a therapeutically effective amount of one or more therapeutic agents and a solvate of a compound of any of the Formulae disclosed herein and a pharmaceutically acceptable carrier.

The disclosure also relates to a pharmaceutical composition of a therapeutically effective amount of one or more therapeutic agents and a stereoisomer of a compound of any of the Formulae disclosed herein and a pharmaceutically acceptable carrier.

The disclosure also relates to a pharmaceutical composition of a therapeutically effective amount of one or more therapeutic agents and a compound selected from Compound A2 and Compound D16 and a pharmaceutically acceptable carrier. The disclosure also relates to a pharmaceutical composition of a therapeutically effective amount of one or more therapeutic agents and a salt of a compound selected from Compound A2 and Compound D16 and a pharmaceutically acceptable carrier. The disclosure also relates to a pharmaceutical composition of a therapeutically effective amount of one or more therapeutic agents and an N-oxide of a compound selected from Compound A2 and Compound D16 and a pharmaceutically acceptable carrier. The disclosure also relates to a pharmaceutical composition of a therapeutically effective amount of one or more therapeutic agents and an N-oxide of salt of a compound selected from Compound A2 and Compound D16 and a pharmaceutically acceptable carrier. The disclosure also relates to a pharmaceutical composition of a therapeutically effective amount of one or more therapeutic agents and a hydrate of a compound selected from Compound A2 and Compound D16 and a pharmaceutically acceptable carrier. The disclosure also relates to a pharmaceutical composition of a therapeutically effective amount of one or more therapeutic agents and a polymorph of a compound selected from Compound A2 and Compound D16 and a pharmaceutically acceptable carrier. The disclosure also relates to a pharmaceutical composition of a therapeutically effective amount of one or more therapeutic agents and a solvate of a compound selected from Compound A2 and Compound D16 and a pharmaceutically acceptable carrier. The disclosure also relates to a pharmaceutical composition of a therapeutically effective amount of one or more therapeutic agents and a stereoisomer of a compound selected from Compound A2 and Compound D16 and a pharmaceutically acceptable carrier.

In the formulae presented herein, the variables can be selected from the respective groups of chemical moieties later defined in the detailed description.

In addition, the disclosure provides methods of synthesizing the foregoing compounds. Following synthesis, a therapeutically effective amount of one or more of the compounds can be formulated with a pharmaceutically acceptable carrier for administration to a mammal, particularly humans, for use in modulating an epigenetic enzyme. In certain embodiments, the compounds of the disclosure are useful for treating, preventing, or reducing the risk of cancer or for the manufacture of a medicament for treating, preventing, or reducing the risk of cancer. Accordingly, the compounds, compositions, or the formulations can be administered, for example, via oral, parenteral, otic, ophthalmic, nasal, or topical routes, to provide an effective amount of the compound to the mammal.

In the present specification, the structural formula of the compound represents a certain isomer for convenience in some cases, but the disclosure includes all isomers, such as geometrical isomers, optical isomers based on an asymmetrical carbon, stereoisomers, tautomers, and the like. In addition, a crystal polymorphism may be present for the compounds represented by the formula. It is noted that any crystal form, crystal form mixture, or anhydride or hydrate thereof is included in the scope of the disclosure. Furthermore, so-called metabolite which is produced by degradation of the present compound in vivo is included in the scope of the disclosure.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the disclosure may be depicted as different chiral isomers or geometric isomers. It should also be understood that when compounds have chiral isomeric or geometric isomeric forms, all isomeric forms are intended to be included in the scope of the disclosure, and the naming of the compounds does not exclude any isomeric forms.

For example, compounds of Formula (I) include those of the following chiral isomers and geometric isomers.

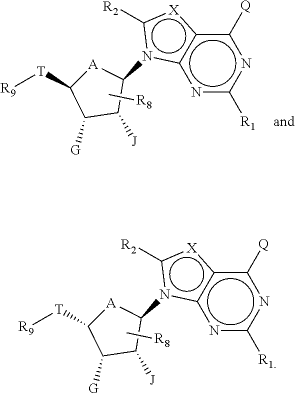

Furthermore, the structures and other compounds discussed in this disclosure include all atropic isomers thereof “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine. Benzimidazoles also exhibit tautomerism, when the benzimidazole contains one or more substituents in the 4, 5, 6 or 7 positions, the possibility of different isomers arises. For example, 2,5-dimethyl-1H-benzo[d]imidazole can exist in equilibrium with its isomer 2,6-dimethyl-1H-benzo[d]imidazole via tautomerization.

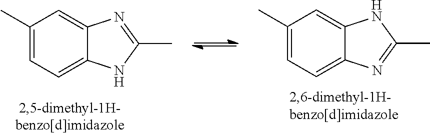

Another example of tautomerism is shown below.

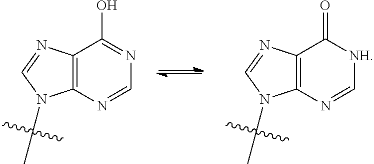 [SMILES: *n1cnc2c(O)ncnc21 ; *n1cnc2c(=O)[nH]cnc21]

It is to be understood that the compounds of the disclosure may be depicted as different tautomers. It should also be understood that when compounds have tautomeric forms, all tautomeric forms are intended to be included in the scope of the disclosure, and the naming of the compounds does not exclude any tautomer form.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Compounds of the disclosure may be crystalline, semi-crystalline, non-crystalline, amorphous, and mesomorphous.

The compounds of any of the Formulae disclosed herein include the compounds themselves, as well as their N-oxides, salts, their solvates, their polymorphs, and their stereoisomers, if applicable. A salt, for example, can be formed between an anion and a positively charged group (e.g., amino) on the compound or inhibitor (e.g., a substituted nucleoside compound such as a substituted purine or 7-deazapurine compound). Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate. Likewise, a salt can also be formed between a cation and a negatively charged group (e.g., carboxylate) on the compound or inhibitor (e.g., a substituted nucleoside compound such as a substituted purine or 7-deazapurine compound). Suitable cations include sodium ion, potassium ion, magnesium ion, calcium ion, and an ammonium cation such as tetramethylammonium ion. The compound or inhibitor (e.g., a substituted nucleoside compound such as a substituted purine or 7-deazapurine compound) also include those salts containing quaternary nitrogen atoms.

Additionally, the compounds of the disclosure, for example, the salts of the compounds, can exist in either hydrated or unhydrated (the anhydrous) form or as solvates with other solvent molecules. Nonlimiting examples of hydrates include hemihydrates, monohydrates, dihydrates, trihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. A hemihydrate is formed by the combination of one molecule of water with more than one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein. For example, all of the compounds represented by Formula (I) are substituted purine compounds or substituted 7-deazapurine compounds, and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The disclosure also provides methods for the synthesis of the compounds of any of the Formulae disclosed herein. The disclosure also provides detailed methods for the synthesis of various disclosed compounds according to the schemes and the Examples described in WO2012/075381, WO2012/075492, WO2012/082436, WO2012/75500, WO2014/026198, WO2014/035140, US2014/0100184, and in J. Med Chem. (2013), 56: p. 8972-8983, the contents of which are hereby incorporated by reference in their entireties.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial unless otherwise specified so long as the invention remains operable. Moreover, two or more steps or actions can be conducted simultaneously.

Throughout the description, where compositions are described as having, including, or comprising specific components, or where processes are described as having, including, or comprising specific process steps, it is contemplated that compositions of the disclosure also consist essentially of, or consist of, the recited components, and that the processes of the disclosure also consist essentially of, or consist of, the recited processing steps. Further, it should be understood that the order of steps or order for performing certain actions are immaterial so long as the invention remains operable. Moreover, two or more steps or actions can be conducted simultaneously.

Compounds suitable for the methods of the disclosure, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described herein.

To further assess a compound's drug-like properties, measurements of inhibition of cytochrome P450 enzymes and phase II metabolizing enzyme activity can also be measured either using recombinant human enzyme systems or more complex systems like human liver microsomes. Further, compounds can be assessed as substrates of these metabolic enzyme activities as well. These activities are useful in determining the potential of a compound to cause drug-drug interactions or generate metabolites that retain or have no useful antimicrobial activity.

To get an estimate of the potential of the compound to be orally bioavailable, one can also perform solubility and Caco-2 assays. The latter is a cell line from human epithelium that allows measurement of drug uptake and passage through a Caco-2 cell monolayer often growing within wells of a 24-well microtiter plate equipped with a 1 micron membrane. Free drug concentrations can be measured on the basolateral side of the monolayer, assessing the amount of drug that can pass through the intestinal monolayer. Appropriate controls to ensure monolayer integrity and tightness of gap junctions are needed. Using this same system one can get an estimate of P-glycoprotein mediated efflux. P-glycoprotein is a pump that localizes to the apical membrane of cells, forming polarized monolayers. This pump can abrogate the active or passive uptake across the Caco-2 cell membrane, resulting in less drug passing through the intestinal epithelial layer. These results are often done in conjunction with solubility measurements and both of these factors are known to contribute to oral bioavailability in mammals. Measurements of oral bioavailability in animals and ultimately in man using traditional pharmacokinetic experiments will determine the absolute oral bioavailability.

Experimental results can also be used to build models that help predict physical-chemical parameters that contribute to drug-like properties. When such a model is verified, experimental methodology can be reduced, with increased reliance on the model predictability.

A composition or combination of the disclosure comprises a compound of Formula (I) (e.g., EPZ-5676 or EPZ-4777), or a pharmaceutically acceptable salt, polymorph, solvate, or stereoisomer thereof, and one or more therapeutic agents. The disclosure provides for the administration of a compound of Formula (I) (e.g., EPZ-5676 or EPZ-4777) or a pharmaceutically acceptable salt, polymorph, solvate, or stereoisomer thereof, and one or more therapeutic agents as a co-formulation or separate formulations, wherein the administration of formulations is simultaneous, sequential, or in alternation. In one embodiment, the one or more therapeutic agents can be an agent that is recognized in the art as being useful to treat the disease or condition being treated by the composition of the disclosure. In another embodiment, the one or more therapeutic agents can be an agent that is not recognized in the art as being useful to treat the disease or condition being treated by the composition of the disclosure. In one aspect, the other therapeutic agents can be an agent that imparts a beneficial attribute to the composition of the disclosure (e.g., an agent that affects the viscosity of the composition). The beneficial attribute to the composition of the disclosure includes, but is not limited to, pharmacokinetic or pharmacodynamic co-action resulting from the combination of a compound of Formula (I) (e.g., EPZ-5676 or EPZ-4777) and one or more therapeutic agents.

In some embodiments, the one or more therapeutic agents can be anticancer agents or chemotherapeutic agents. For example, the one or more therapeutic agents can be selected from Ara-C, Daunorubicin, Azacitidine, Decitabine, Panobinostat, Vidaza, Mitoxantrone, Methotrexate, Mafosfamide, Prednisolone, Vincristine, Lenalidomide, Hydroxyurea, Menin-MLL inhibitor MI-2, JQ1, IBET151, Vorinostat, Quizartinib, Midostaurin, Tranylcypromine, LSD1 inhibitor II, Navitoclax, Velcade, SRT-1720, Furazolidone, Fludarabine, Mercaptopurine, Obatoclax, ABT-199, Trametinib, Clofarabine, Ibrutinib, Palbociclib, AZ20, MK2206, BEZ235, T0070907, Romidepsin, Tipifarnib, Volasertib, Compound E10, 10-Hydroxycamptothecin, ABT-737, Alitretinoin, AT7867, Auranofin, AZD 8055, AZD6244, Baricitinib, BEP800, Bexarotene, BIX01294, Bleomycin Sulfate, BMN 673, BMS 345541, BMS-754807, BX-912, C 646, CAL-101, CAPE, Cerivastatin Sodium, Chlorambucil, Cisplatin, CPI-203, Dabrafenib, GSK-LSD1, Erlotinib Hydrochloride, Etoposide, Everolimus, Fostamatinib disodium, GDC-0941, Go 6976, GSK2656157, IKK-2 Inhibitor VIII, Irinotecan Hydrochloride, JNJ 26854165, KU 0063794, Lapatinib, LB42708, LDN 57444, LEE011, LY2603618, Melphalan, Menadione, Methylprednisolone, Mitomycin C, MK-2206, MLN2238, MS 436, MS-275, NKH 477, NU 7441, Nutlin-3, Olaparib, OTX015, Oxaliplatin, Papaverine Hydrochloride, Parthenolide, PHA-793887, Pomalidomide, Raloxifene Hydrochloride, SB-505124, SCH772984, SGC-CBP30, SMER 3, Sorafenib, SRT1720, TANSHINONE IIA, Temsirolimus, Thiostrepton, Thiotepa, Topotecan Hydrochloride, Tretinoin, Triciribine, UNC 0646, VE-821, XL147, or functional analogs, derivatives, prodrugs, and metabolites thereof. Preferably, the therapeutic agent is Ara-C, Azacitidine, or Daunorubicin or functional analogs, derivatives, prodrugs, and metabolites thereof. Alternatively, the therapeutic agent is a standard of care agent. See, e.g., Klaus et al., J Pharmacol Exp Ther 350:1-11, (September 2014), the content of which are hereby incorporated by reference in its entirety.

In some embodiments, the one or more therapeutic agents include an immunomodulatory drug such as Lenalidomide.

In some embodiments, the one or more therapeutic agents include a SIRT1 activator such as SRT-1720.

In some embodiments, the one or more therapeutic agents include an antibiotic such as Furazolidone.

In some embodiments, the one or more therapeutic agents include a topoisomerase inhibitor (e.g., Mitoxantrone), a hypomethylating agent (e.g., Decitabine or Vidaza), a Menin inhibitor (e.g., MI-2), a Bromodomain inhibitor (e.g., IBET-151 and JQ1), an HDAC inhibitor (e.g., Panobinostat and Vorinostat), a Bcl-2 inhibitor (e.g., Navitoclax, Obatoclax, or ABT-199), a MEK1/2 inhibitor (e.g., Trametinib), a BTK inhibitor (e.g., Ibrutinib), a CDK4/6 inhibitor (e.g., Palbociclib), a FLT inhibitor (e.g., Quizartinib or Midostaurin), an HDM inhibitor (e.g., Tranylcypromine and LSD1 inhibitor II) an AML standard of care drug (such as Ara-C, Daunorubicin, and Mercaptopurine), an ALL standard of care drug (such as Mitoxantrone, Methotrexate, Mafosfamide, Prednisolone, and Vincristine), an ALL/AML standard of care drug (e.g., Fludarabine), a DNMT inhibitor (such as Azacitidine and Decitabine), an immunomodulatory drug (e.g., Lenalidomide), a proteasome inhibitor (e.g., Velcade), an antimetabolite (e.g., Hydroxyurea and Clofarabine), a SIRT1 activator (e.g., SRT-1720), an antibiotic (e.g., a nitrofuran such as Furazolidone), an ATR inhibitor (e.g., AZ20 and VE-821), an AKT inhibitor, such as an AKT1 inhibitor or a pan-AKT allosteric inhibitor (e.g., MK2206), a dual PI3K/MTOR inhibitor (e.g., BEZ235), a PPAR antagonist (e.g., GW9662, or T0070907), an EZH2 (enhancer of zeste 2 polycomb repressive complex 2 subunit) inhibitor (e.g.,

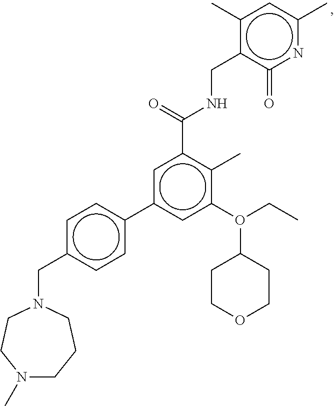

herein referred to as Compound E10), a Farnesyl Transferase inhibitor (e.g., Tipifarnib), a PLK1 inhibitor (e.g., Volasertib), or a combination of any of the therapeutic agents disclosed herein.

In some embodiments, the one or more therapeutic agents include a Bromodomain inhibitor (e.g., IBET-151), a Menin inhibitor (e.g., MI-2).

In some embodiments, the one or more therapeutic agents include an HDM inhibitor (e.g., Tranylcypromine).

In some embodiments, the one or more therapeutic agents include Mafosfamide.

In some embodiments, the one or more therapeutic agents include a CDK4/6 inhibitor (e.g., Palbociclib).

In some embodiments, the one or more therapeutic agents include one or more compounds included in Tables 4-8 (e.g., those showing an additive or synergistic effect in combination with Compound A2 in Molm13 and/or MV4-11 cells).

In some embodiments, the one or more therapeutic agents include one or more compounds included in Tables 4-8 which show a synergistic effect in combination with Compound A2 in Molm13 and/or MV4-11 cells.

The therapeutic agents set forth below are for illustrative purposes and not intended to be limiting. The disclosure includes at least one therapeutic agent selected from the lists below. The disclosure can include more than one therapeutic agent, e.g., two, three, four, or five therapeutic agents such that the composition of the disclosure can perform its intended function.

In one embodiment, the other therapeutic agent is an anticancer agent. In one embodiment, the anticancer agent is a compound that affects histone modifications, such as an HDAC inhibitor. In certain embodiments, an anticancer agent is selected from the group consisting of chemotherapeutics (such as 2CdA, 5-FU, 6-Mercaptopurine, 6-TG, Abraxane™, Accutane®, Actinomycin-D, Adriamycin®, Alimta®, all-trans retinoic acid, amethopterin, Ara-C, Azacitidine, BCNU, Blenoxane®, Camptosar®, CeeNU®, Clofarabine, Clolar™, Cytoxan®, daunorubicin hydrochloride, DaunoXome®, Dacogen®, DIC, Ellence®, Eloxatin®, Emcyt®, etoposide phosphate, Fludara®, FUDR®, Gemzar®, Gleevec®, hexamethylmelamine, Hycamtin®, Hydrea®, Idamycin®, Ifex®, ixabepilone, Ixempra®, L-asparaginase, Leukeran®, liposomal Ara-C, L-PAM, Lysodren, Matulane®, mithracin, Mitomycin-C, Myleran®, Navelbine®, Neutrexin®, nilotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Trisenox®, Valstar®, Velban®, Vidaza™, vincristine sulfate, VM 26, Xeloda® and Zanosar®); biologics (such as Alpha Interferon, Bacillus Calmette-Guerin, Bexxar®, Campath®, Ergamisol®, Erlotinib, Herceptin®, Interleukin-2, Iressa®, lenalidomide, Mylotarg®, Ontak®, Pegasys®, Revlimid®, Rituxan®, Tarceva™, Thalomid®, Tykerb®, Velcade® and Zevalin™); corticosteroids, (such as dexamethasone sodium phosphate, DeltaSone® and Delta-Cortef®); hormonal therapies (such as Arimidex®, Aromasin®, Casodex®, Cytadren®, Eligard®, Eulexin®, Evista®, Faslodex®, Femara®, Halotestin®, Megace®, Nilandron®, Nolvadex®, Plenaxis™ and Zoladex®); and radiopharmaceuticals (such as Iodotope®, Metastron®, Phosphocol® and Samarium SM-153).

In another embodiment, the other therapeutic agent is a chemotherapeutic agent (also referred to as an anti-neoplastic agent or anti-proliferative agent), selected from the group including an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug or any chemotherapeutic, anti-neoplastic or anti-proliferative agent listed at the World Wide Web (www) cancer.org/docroot/cdg/cdg_0.asp.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine131 tositumomab (Bexxar); alemtuzumab (Campath); ibritumomab (Zevalin; In-111; Y-90 Zevalin); gemtuzumab (Mylotarg); eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569.

Exemplary HER2 inhibitors include, but are not limited to, trastuzumab (Herceptin); lapatinib (Tykerb) or AC-480.

Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur) ; fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus Torisel); rapamune, ridaforolimus; or AP23573.

Exemplary multi-kinase inhibitors include, but are not limited to, sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/easudil hydrochloride; flavopiridol; Pkc412; bryostatin; KAI-9803; SF1126; or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, paclitaxel, docetaxel, vincristine, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur—0.4 M 5-chloro-2,4-dihydroxypyrimidine—1 M potassium oxonate), or lovastatin.

In another aspect, the other therapeutic agent is a chemotherapeutic agent or a cytokine such as G-CSF (granulocyte colony stimulating factor).

In yet another aspect, the other therapeutic agents can be standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™), CHOP (cyclophosphamide, hydroxydaunorubicin, oncovin, and prednisone or prednisolone), R-CHOP (rituximab, cyclophosphamide, hydroxydaunorubicin, oncovin, prednisone or prednisolone), or CMFP (cyclophosphamide, methotrexate, 5-fluorouracil and prednisone).

In another aspect, the other therapeutic agents can be an inhibitor of an enzyme, such as a receptor or non-receptor kinase. Receptor and non-receptor kinases are, for example, tyrosine kinases or serine/threonine kinases. Kinase inhibitors described herein are small molecules, polynucleic acids, polypeptides, or antibodies.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-β, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

Exemplary serine/threonine kinase inhibitors include, but are not limited to, Rapamune (targets mTOR/FRAP1), Deforolimus (targets mTOR), Certican/Everolimus (targets mTOR/FRAP1), AP23573 (targets mTOR/FRAP1), Eril/Fasudil hydrochloride (targets RHO), Flavopiridol (targets CDK), Seliciclib/CYC202/Roscovitrine (targets CDK), SNS-032/BMS-387032 (targets CDK), Ruboxistaurin (targets PKC), Pkc412 (targets PKC), Bryostatin (targets PKC), KAI-9803 (targets PKC), SF1126 (targets PI3K), VX-680 (targets Aurora kinase), Azd1152 (targets Aurora kinase), Any-142886/AZD-6244 (targets MAP/MEK), SCIO-469 (targets MAP/MEK), GW681323 (targets MAP/MEK), CC-401 (targets JNK), CEP-1347 (targets JNK), and PD 332991 (targets CDK).

In one embodiment, a composition of the disclosure includes a compound of Formula (I) (e.g., EPZ-5676 or EPZ-4777) or a pharmaceutically acceptable salt thereof, and one or more anticancer agents. Anticancer agents include, for example, Ara-C, Daunorubicin, Decitabine, Vidaza, Mitoxantrone, JQ1, IBET151, Panobinostat, Vorinostat, Quizartinib, Midostaurin, Tranylcypromine, LSD1 inhibitor II, Navitoclax, or functional analogs, derivatives, prodrugs, and metabolites thereof.

In certain embodiments, the one or more other therapeutic agents are selected from inhibitors in the RAS-RAF-MEK-ERK pathway (also known as the MAPK (ERK) pathway). The MAPK (ERK) pathway involves several proteins that can be targeted by inhibitors. For example, inhibitors that target any one or more of active, inactive, or mutated forms of RAS (small G protein), BRAF (MAPKKK), MEK (MAPKK), and ERK (MAPK) can be used in combination with any one or more DOT1L inhibitors disclosed herein. Examples of inhibitors in the MAPK (ERK) pathway include but are not limited to, MEK1 and/or MEK2 inhibitors (e.g., MEK162, Selumetinib, Trametinib, cobimetinib, CI-1040, PD035901, AZD6244, R05126766, GDC-0623, or PD0325901); ERK inhibitors (e.g., SCH772984, GDC0994, Ulixertinib, VTX11e); and RAF inhibitors (sorafenib, RAF265, GDC-0879, PLX-4032, dabrafenib, SB590885, PLX4720, XL281, encorafenib, vemurafenib, MLN2480, or TAK-632). For example, RAS-RAF-MEK-ERK inhibitors suitable for the combinations and methods disclosed herein include those that target specific MAPK (ERK) pathway mutants, such as inhibitors that target the BRAF V600E mutant (e.g., Dabrafenib, LGX818, or Vemurafenib). More examples of inhibitors in the RAS-RAF-MEK-ERK pathway are described in, e.g., Nature Reviews Drug Discovery (2014) 13, 928-942, Leukemia (2003) 17, 1263-1293; and Pharmacy and Therapeutics (2013) 38(2): 96-98, 105-108; the contents of each of which are incorporated herein by reference in their entireties.

In certain embodiments, the one or more inhibitors in the RAS-RAF-MEK-ERK pathway suitable to be used in combination with any one or more DOT1L inhibitors (e.g., EPZ-5676 or EPZ-4777) disclosed herein are selected from MEK162, Selumetinib, Trametinib, SCH772984, GDC0994, Ulixertinib, Sorafenib and RAF265.

The disclosure provides methods for combination therapy in which a composition comprising a compound of Formula (I) (e.g., EPZ-5676 or EPZ-4777) or a pharmaceutically acceptable salt, polymorph, solvate, or stereoisomer thereof, and one or more other therapeutic agents are administered to a subject in need for treatment of a disease or cancer. The combination therapy can also be administered to cancer cells to inhibit proliferation or induce cell death.

The disclosure includes the combination therapy of administering a compound of Formula (I) (e.g., EPZ-5676 or EPZ-4777) or a pharmaceutically acceptable salt, polymorph, solvate, or stereoisomer thereof, and anticancer agents, where the anticancer agents are selected from Ara-C, Daunorubicin, Decitabine, Vidaza, Mitoxantrone, JQ1, IBET151, Panobinostat, Vorinostat, Quizartinib, Midostaurin, Tranylcypromine, LSD1 inhibitor II, trametinib, and Navitoclax, or functional analogs, derivatives, prodrugs, and metabolites thereof.

In one aspect, a compound of Formula (I) (e.g. EPZ-5676 or EPZ-4777) or a pharmaceutically acceptable salt, polymorph, solvate, or stereoisomer thereof and one or more therapeutic agents are administered simultaneously or sequentially.

In one aspect, a compound of Formula (I) (e.g. EPZ-5676 or EPZ-4777) or a pharmaceutically acceptable salt, polymorph, solvate, or stereoisomer thereof is administered prior to administration of the composition of the disclosure comprising a compound of Formula (I) (e.g. EPZ-5676 or EPZ-4777) or a pharmaceutically acceptable salt, polymorph, solvate, or stereoisomer thereof, and one or more therapeutic agents.

In one aspect, one or more therapeutic agents are administered prior to administration of a composition of the disclosure comprising a compound of Formula (I) (e.g. EPZ-5676 or EPZ-4777) or a pharmaceutically acceptable salt, polymorph, solvate, or stereoisomer thereof and one or more therapeutic agents. The one or more therapeutic agents are administered either in a single composition or in two or more compositions, e.g. administered simultaneously, sequentially, or in alternation.

In one aspect, a compound of Formula (I) (e.g. EPZ-5676 or EPZ-4777) or a pharmaceutically acceptable salt, polymorph, solvate, or stereoisomer thereof is administered prior to administration of one or more therapeutic agents, such that the one or more therapeutic agents are administered either in a single composition or in two or more compositions, e.g. administered simultaneously, sequentially, or in alternation.

In one aspect, one or more therapeutic agents are administered prior to administration of a compound of Formula (I) (e.g. EPZ-5676 or EPZ-4777) or a pharmaceutically acceptable salt, polymorph, solvate, or stereoisomer thereof. The one or more therapeutic agents are administered either in a single composition or in two or more compositions, e.g. administered simultaneously, sequentially, or in alternation.

In one aspect, a compound of Formula (I) (e.g. EPZ-5676 or EPZ-4777) or a pharmaceutically acceptable salt, polymorph, solvate, or stereoisomer thereof and the one or more therapeutic agents are administered sequentially. It should be appreciated that the one or more therapeutic agents can be administered one or more hours, or one or more days after a compound of Formula (I) (e.g. EPZ-5676 or EPZ-4777) or a pharmaceutically acceptable salt, polymorph, solvate, or stereoisomer thereof is administered. Alternatively, the one or more therapeutic agents can be administered one or more hours, or one or more days prior to a compound of Formula (I) (e.g. EPZ-5676 or EPZ-4777) or a pharmaceutically acceptable salt, polymorph, solvate, or stereoisomer thereof is administered.

In some embodiments, the one or more therapeutic agents are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of a compound of Formula (I) (e.g. EPZ-5676 or EPZ-4777) or a pharmaceutically acceptable salt, polymorph, solvate, or stereoisomer thereof. In some embodiments, the one or more therapeutic agents are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more prior to the administration of a compound of Formula (I) (e.g. EPZ-5676 or EPZ-4777) or a pharmaceutically acceptable salt, polymorph, solvate, or stereoisomer thereof.

For example, a MEK inhibitor (e.g., trametinib) and EPZ-5676 are administered simultaneously or sequentially. For example, a MEK inhibitor (e.g., trametinib) is administered 1-21 days (e.g., 3-14 days, 4-10 days, 7-8 days, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days) after the administration of EPZ-5676 or a pharmaceutically acceptable salt, polymorph, solvate, or stereoisomer thereof. As another example, a MEK inhibitor (e.g., trametinib) is administered 1-21 days (e.g., 3-14 days, 4-10 days, 7-8 days, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days) prior to the administration of EPZ-5676 or a pharmaceutically acceptable salt, polymorph, solvate, or stereoisomer thereof.

For example, an ERK inhibitor (e.g., SCH772984) and EPZ-5676 are administered simultaneously or sequentially. For example, an ERK inhibitor (e.g., SCH772984) is administered 1-21 days (e.g., 3-14 days, 4-10 days, 7-8 days, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days) after the administration of EPZ-5676 or a pharmaceutically acceptable salt, polymorph, solvate, or stereoisomer thereof. As another example, an ERK inhibitor (e.g., SCH772984) is administered 1-21 days (e.g., 3-14 days, 4-10 days, 7-8 days, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days) prior to the administration of EPZ-5676 or a pharmaceutically acceptable salt, polymorph, solvate, or stereoisomer thereof.

In some embodiments, the composition comprising a compound of Formula (I) or a pharmaceutically acceptable salt, polymorph, solvate, or stereoisomer thereof and one or more therapeutic agents are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of a compound of Formula (I) (e.g. EPZ-5676 or EPZ-4777) or a pharmaceutically acceptable salt, polymorph, solvate, or stereoisomer thereof.

In some embodiments, the composition comprising a compound of Formula (I) (e.g. EPZ-5676 or EPZ-4777) or a pharmaceutically acceptable salt, polymorph, solvate, or stereoisomer thereof and one or more therapeutic agents are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of the one or more therapeutic agents.

In some embodiments, the one or more therapeutic agents are administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or more after the administration of a compound of Formula (I) (e.g. EPZ-5676 or EPZ-4777) or a pharmaceutically acceptable salt, polymorph, solvate, or stereoisomer thereof. In some embodiments, the one or more therapeutic agents are administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or more prior to the administration of a compound of Formula (I) (e.g. EPZ-5676 or EPZ-4777) or a pharmaceutically acceptable salt, polymorph, solvate, or stereoisomer thereof. In some embodiments, the composition comprising a compound of Formula (I) (e.g. EPZ-5676 or EPZ-4777) or a pharmaceutically acceptable salt, polymorph, solvate, or stereoisomer thereof and one or more therapeutic agents are administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or more after the administration of a compound of Formula (I) (e.g. EPZ-5676 or EPZ-4777) or a pharmaceutically acceptable salt, polymorph, solvate, or stereoisomer thereof.

In some embodiments, the composition comprising a compound of Formula (I) (e.g. EPZ-5676 or EPZ-4777) or a pharmaceutically acceptable salt, polymorph, solvate, or stereoisomer thereof and one or more therapeutic agents are administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or more after the administration of the one or more therapeutic agents.

It should be appreciated that the one or more therapeutic agents or the composition comprising a compound of Formula (I) (e.g. EPZ-5676 or EPZ-4777) or a pharmaceutically acceptable salt, polymorph, solvate, or stereoisomer thereof and one or more therapeutic agents, can be administered to a subject after the level in a subject of a compound of Formula (I) (e.g. EPZ-5676 or EPZ-4777) or a pharmaceutically acceptable salt, polymorph, solvate, or stereoisomer thereof that has been administered to the subject has decreased. Thus, for instance, a compound of Formula (I) (e.g. EPZ-5676 or EPZ-4777) or a pharmaceutically acceptable salt, polymorph, solvate, or stereoisomer thereof is administered to a subject and the one or more therapeutic agents are administered after the level of administered compound of Formula (I) (e.g. EPZ-5676 or EPZ-4777) or a pharmaceutically acceptable salt, polymorph, solvate, or stereoisomer thereof is less than 90% of the initial level, less than 80% of the initial level, less than 70% of the initial level, less than 60% of the initial level, less than 50% of the initial level, less than 40% of the initial level, less than 30% of the initial level, less than 20% of the initial level or less than 10% of the initial level. In some embodiments, a compound of Formula (I) (e.g. EPZ-5676 or EPZ-4777) or a pharmaceutically acceptable salt, polymorph, solvate, or stereoisomer thereof that has been administered to a subject can no longer be detected in a subject prior to administration of the one or more therapeutic agents.

It should be appreciated that a compound of Formula (I) (e.g. EPZ-5676 or EPZ-4777) or a pharmaceutically acceptable salt, polymorph, solvate, or stereoisomer thereof or the composition comprising a compound of Formula (I) (e.g. EPZ-5676 or EPZ-4777) or a pharmaceutically acceptable salt, polymorph, solvate, or stereoisomer thereof and one or more therapeutic agents, can be administered to a subject after the level(s) in a subject one or more therapeutic agents that have been administered to the subject has decreased. For example, one or more therapeutic agents are administered to a subject and a compound of Formula (I) (e.g. EPZ-5676 or EPZ-4777) or a pharmaceutically acceptable salt, polymorph, solvate, or stereoisomer thereof is administered after the level of administered one or more therapeutic agents is less than 90% of the initial level, less than 80% of the initial level, less than 70% of the initial level, less than 60% of the initial level, less than 50% of the initial level, less than 40% of the initial level, less than 30% of the initial level, less than 20% of the initial level or less than 10% of the initial level. In some embodiments, one or more therapeutic agents that have been administered to a subject can no longer be detected in a subject prior to administration of a compound of Formula (I) (e.g., EPZ-5676 or EPZ-4777) or a pharmaceutically acceptable salt, polymorph, solvate, or stereoisomer thereof.

For example, the compound of Formula (I) has the formula

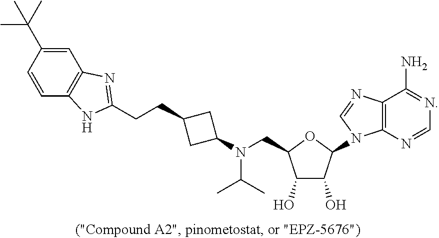 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

For example, the compound of Formula (I) has the formula

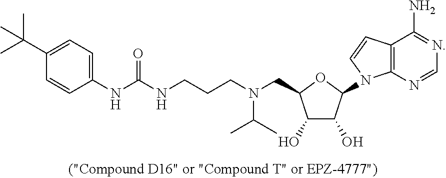 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

Any of the above compounds include its pharmaceutically acceptable salt, polymorph, solvate, or stereoisomer thereof.

In one aspect, the disclosure provides methods for sensitizing or priming a subject to administration of one or more therapeutic agents (e.g., anti-cancer agents). In some embodiments, a subject is sensitized or primed to one or more therapeutic agents (e.g., anti-cancer agents) by administering a compound of Formula (I) (e.g., EPZ-5676 or EPZ-4777) or a pharmaceutically acceptable salt, polymorph, solvate, or stereoisomer thereof. Thus, in one aspect, a compound of Formula (I) (e.g., EPZ-5676 or EPZ-4777) or a pharmaceutically acceptable salt, polymorph, solvate, or stereoisomer thereof is administered to a subject resulting in the sensitization or priming of the subject after which the one or more therapeutic agents (e.g., anti-cancer agents) or the composition comprising a compound of Formula (I) (e.g., EPZ-5676 or EPZ-4777) or a pharmaceutically acceptable salt, polymorph, solvate, or stereoisomer thereof and one or more therapeutic agents, are administered. While not being limited to a specific mechanism it is thought that a subject is sensitized by the administration of a compound of Formula (I) (e.g., EPZ-5676 or EPZ-4777) or a pharmaceutically acceptable salt, polymorph, solvate, or stereoisomer thereof, through a durable altered chromatin state caused by the administration of administering a compound of Formula (I) (e.g., EPZ-5676 or EPZ-4777) or a pharmaceutically acceptable salt, polymorph, solvate, or stereoisomer thereof. In some embodiments, the durable altered chromatin state is decreased histone methylation. In some embodiments the decreased chromatin methylation is decreased methylation of H3K79. In some embodiments, the durable altered chromatin state is present at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of a compound of Formula (I) (e.g., EPZ-5676 or EPZ-4777) or a pharmaceutically acceptable salt, polymorph, solvate, or stereoisomer thereof.

In one aspect, the disclosure provides methods for sensitizing or priming a subject to administration of a compound of Formula (I) (e.g., EPZ-5676 or EPZ-4777) or a pharmaceutically acceptable salt, polymorph, solvate, or stereoisomer thereof. In some embodiments, a subject is sensitized or primed for responding to a compound of Formula (I) (e.g., EPZ-5676 or EPZ-4777) or a pharmaceutically acceptable salt, polymorph, solvate, or stereoisomer thereof by administering one or more therapeutic agents (e.g., anti-cancer agents). Thus, in one aspect, one or more therapeutic agents or the composition comprising a compound of Formula (I) (e.g., EPZ-5676 or EPZ-4777) or a pharmaceutically acceptable salt, polymorph, solvate, or stereoisomer thereof and one or more therapeutic agents, are administered to a subject prior to the administration of a compound of Formula (I) (e.g., EPZ-5676 or EPZ-4777) or a pharmaceutically acceptable salt, polymorph, solvate, or stereoisomer thereof, resulting in the sensitization or priming of the subject. Consequently the subject is more sensitive to a compound of Formula (I) or a pharmaceutically acceptable salt, polymorph, solvate, or stereoisomer thereof.

In some embodiments, the administration of a compound of Formula (I) (e.g., EPZ-5676 or EPZ-4777) or a pharmaceutically acceptable salt, polymorph, solvate, or stereoisomer thereof results in a biological effect prior to the administration of the one or more therapeutic agents (e.g., anti-cancer agents) or the composition comprising a compound of Formula (I) (e.g., EPZ-5676 or EPZ-4777) or a pharmaceutically acceptable salt, polymorph, solvate, or stereoisomer thereof and one or more therapeutic agents. In some embodiments, the one or more therapeutic agents (e.g., anti-cancer agents) are not administered until 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of a compound of Formula (I) (e.g., EPZ-5676 or EPZ-4777) or a pharmaceutically acceptable salt, polymorph, solvate, or stereoisomer thereof has resulted in a biological effect. In some embodiments, the biological effect is a reduction of H3K79 methyl mark, maturation or induction of blast cells, apoptosis of leukemic blast cells, resolution of fevers, cachexia or leukemia cutis and/or restoration of normal haemoatopoiesis. It should be appreciated that more than one biological effect may result from the administration of a compound of Formula (I) (e.g., EPZ-5676 or EPZ-4777) or a pharmaceutically acceptable salt, polymorph, solvate, or stereoisomer thereof. In some embodiments, the biological effect is a reduction of H3K79 methyl mark. In some embodiments, the biological effect is a reduction of H3K79 methyl mark to at least 90%, 80%, 70%, 60%, 50%, 40%, 30%, 20%, 10% or less compared to untreated control levels. In some embodiments, the H3K79 methyl mark must be at least 90%, 80%, 70%, 60%, 50%, 40%, 30%, 20%, 10% or less compared to untreated control levels prior to the addition of the one or more therapeutic agents. In some embodiments, the biological effect is the maturation or differentiation of leukemic blast cells. In some embodiments, at least 20% of leukemic blast cells have undergone maturation or differentiation, at least 50% of leukemic blast cells have undergone maturation or differentiation, or at least 80% of leukemic blast cells have undergone maturation or differentiation prior to the addition of the one or more therapeutic agents. In some embodiments, the biological effect is the apoptosis of leukemic blast cells. In some embodiments, at least 20%, 30%, 40%, 50%, 60%, 70%, 80%, or 90% of the leukemic blast cells undergo cell death or apoptosis prior to administration of the one or more therapeutic agents. In some embodiments, the biological effect is the resolution of fever, resolution of cachexia and/or resolution of leukemia cutis. In some embodiments, fever, cachexia and/or leukemia cutis is resolved prior to administration of the one or more therapeutic agents. In some embodiments, the biological effect is the restoration of normal haematopoiesis. In some embodiments, normal haematopoiesis is restored prior to administration of the one or more therapeutic agents.

In some embodiments, the administration of one or more therapeutic agents (e.g., anti-cancer agents) results in a biological effect prior to the administration of a compound of Formula (I) (e.g., EPZ-5676 or EPZ-4777) or a pharmaceutically acceptable salt, polymorph, solvate, or stereoisomer thereof or the composition comprising a compound of Formula (I) (e.g., EPZ-5676 or EPZ-4777) or a pharmaceutically acceptable salt, polymorph, solvate, or stereoisomer thereof and one or more therapeutic agents. In some embodiments, a compound of Formula (I) (e.g., EPZ-5676 or EPZ-4777) or a pharmaceutically acceptable salt, polymorph, solvate, or stereoisomer thereof is not administered until 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of one or more therapeutic agents have resulted in a biological effect. In some embodiments, the biological effect is a reduction of H3K79 methylmark, maturation or induction of blast cells, apoptosis of leukemic blast cells, resolution of fevers, cachexia or leukemia cutis and/or restoration of normal haemoatopoiesis. It should be appreciated that more than one biological effect may result from the administration of one or more therapeutic agents. In some embodiments, the biological effect is a reduction of H3K79 methyl mark. In some embodiments, the biological effect is a reduction of H3K79 methyl mark to at least 90%, 80%, 70%, 60%, 50%, 40%, 30%, 20%, 10% or less compared to untreated control levels. In some embodiments, the H3K79 methyl mark must be at least 90%, 80%, 70%, 60%, 50%, 40%, 30%, 20%, 10% or less compared to untreated control levels prior to the addition of a compound of Formula (I) (e.g., EPZ-5676 or EPZ-4777) or a pharmaceutically acceptable salt, polymorph, solvate, or stereoisomer thereof.

In some embodiments, the biological effect is the maturation or differentiation of leukemic blast cells. In some embodiments, at least 20% of leukemic blast cells have undergone maturation or differentiation, at least 50% of leukemic blast cells have undergone maturation or differentiation, or at least 80% of leukemic blast cells have undergone maturation or differentiation prior to the addition of a compound of Formula (I) (e.g., EPZ-5676 or EPZ-4777) or a pharmaceutically acceptable salt, polymorph, solvate, or stereoisomer thereof.

In some embodiments, the biological effect is the apoptosis of leukemic blast cells. In some embodiments, at least 20%, 30%, 40%, 50%, 60%, 70%, 80%, or 90% of the leukemic blast cells undergo cell death or apoptosis prior to administration of a compound of Formula (I) (e.g., EPZ-5676 or EPZ-4777) or a pharmaceutically acceptable salt, polymorph, solvate, or stereoisomer thereof. In some embodiments, the biological effect is the resolution of fever, resolution of cachexia and/or resolution of leukemia cutis. In some embodiments, fever, cachexia and/or leukemia cutis is resolved prior to administration of a compound of Formula (I) (e.g., EPZ-5676 or EPZ-4777) or a pharmaceutically acceptable salt, polymorph, solvate, or stereoisomer thereof. In some embodiments, the biological effect is the restoration of normal haematopoiesis. In some embodiments, normal haematopoiesis is restored prior to administration of a compound of Formula (I) (e.g., EPZ-5676 or EPZ-4777) or a pharmaceutically acceptable salt, polymorph, solvate, or stereoisomer thereof.

In some embodiments, a subject is evaluated after the administration of a compound of Formula (I) (e.g., EPZ-5676 or EPZ-4777) or a pharmaceutically acceptable salt, polymorph, solvate, or stereoisomer thereof for any biological effects prior to administration of one or more therapeutic agents (e.g., anti-cancer agents) or the composition comprising a compound of Formula (I) (e.g., EPZ-5676 or EPZ-4777) or a pharmaceutically acceptable salt, polymorph, solvate, or stereoisomer thereof and one or more therapeutic agents. In some embodiments, the one or more therapeutic agents are administered only if the evaluated biological effect has reached a certain predetermined level or activity. In some embodiments, the biological effect is maturation or induction of blast cells, apoptosis of leukemic blast cells, resolution of fever, cachexia or leukemia cutis and/or restoration of normal haemoatopoiesis. In some embodiments, the biological effect is a durable altered chromatin state. In some embodiments, the durable altered chromatin state is decreased histone methylation. In some embodiments the decreased chromatin methylation is decreased methylation of H3K79. In some embodiments, the durable altered chromatin state is present at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of a compound of Formula (I) (e.g., EPZ-5676 or EPZ-4777) or a pharmaceutically acceptable salt, polymorph, solvate, or stereoisomer thereof.

In some embodiments, a subject is evaluated after the administration of one or more therapeutic agents (e.g., anti-cancer agents) for any biological effects prior to administration of a compound of Formula (I) (e.g., EPZ-5676 or EPZ-4777) or a pharmaceutically acceptable salt, polymorph, solvate, or stereoisomer thereof or the composition comprising a compound of Formula (I) (e.g., EPZ-5676 or EPZ-4777) or a pharmaceutically acceptable salt, polymorph, solvate, or stereoisomer thereof and one or more therapeutic agents, In some embodiments, a compound of Formula (I) (e.g., EPZ-5676 or EPZ-4777) or a pharmaceutically acceptable salt, polymorph, solvate, or stereoisomer thereof is administered only if the evaluated biological effect has reached a certain predetermined level or activity. In some embodiments, the biological effect is maturation or induction of blast cells, apoptosis of leukemic blast cells, resolution of fever, cachexia or leukemia cutis and/or restoration of normal haemoatopoiesis. In some embodiments, the biological effect is a durable altered chromatin state. In some embodiments, the durable altered chromatin state is decreased histone methylation. In some embodiments the decreased chromatin methylation is decreased methylation of H3K79. In some embodiments, the durable altered chromatin state is present at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of one or more therapeutic agents.

In certain aspects of the invention, the sensitization or priming by a compound of Formula (I) (e.g., EPZ-5676 or EPZ-4777) results in the need for lower therapeutically effective amounts of the sequential therapeutic agent. It should be appreciated that in certain embodiments the sensitization would result in a synergistic effect as described herein between the compound of Formula (I) and the therapeutic agent, such as a standard of care agent.

In certain aspects of the invention, the sensitization or priming by one or more therapeutic agents results in the need for lower therapeutically effective amounts of the sequential administration of a compound of Formula (I) (e.g., EPZ-5676 or EPZ-4777) or a pharmaceutically acceptable salt, polymorph, solvate, or stereoisomer thereof or a composition of the disclosure. It should be appreciated that in certain embodiments the sensitization would result in a synergistic effect as described herein between the compound of Formula (I) and the therapeutic agent, such as a standard of care agent.

In one aspect, a compound of Formula (I) (e.g., EPZ-5676 or EPZ-4777) or a pharmaceutically acceptable salt, polymorph, solvate, or stereoisomer thereof is administered continuously. In some embodiments, the compound of Formula (I) or a pharmaceutically acceptable salt, polymorph, solvate, or stereoisomer thereof is administered continuously for at least 7, 14, 21, 28, 35, 42, 47, 56 or 64 days. In some embodiments, the compound of Formula (I) or a pharmaceutically acceptable salt, polymorph, solvate, or stereoisomer thereof is administered without a drug holiday.

In one aspect, a compound of Formula (I) (e.g., EPZ-5676 or EPZ-4777) or a pharmaceutically acceptable salt, polymorph, solvate, or stereoisomer thereof and one or more therapeutic agents are administered simultaneously or sequentially. In some embodiments, a compound of Formula (I) (e.g., EPZ-5676 or EPZ-4777) or a pharmaceutically acceptable salt, polymorph, solvate, or stereoisomer thereof and the one or more therapeutic agents are administered continuously. In some embodiments, the compound of Formula (I) or a pharmaceutically acceptable salt, polymorph, solvate, or stereoisomer thereof and the one or more therapeutic agents are administered continuously for at least 7, 14, 21, 28, 35, 42, 47, 56 or 64 days. In some embodiments, the compound of Formula (I) or a pharmaceutically acceptable salt, polymorph, solvate, or stereoisomer thereof and the one or more therapeutic agents are administered without a drug holiday.

In one aspect, a compound of Formula (I) (e.g., EPZ-5676 or EPZ-4777) or a pharmaceutically acceptable salt, polymorph, solvate, or stereoisomer thereof is administered continuously while the one or more therapeutic agents are not administered continuously. In some embodiments, the compound of Formula (I) or a pharmaceutically acceptable salt, polymorph, solvate, or stereoisomer thereof is administered continuously for at least 7, 14, 21, 28, 35, 42, 47, 56 or 64 days while the one or more therapeutic agents is not administered continuously for at least 7, 14, 21, 28, 35, 42, 47, 56 or 64 days. In some embodiments, the compound of Formula (I) or a pharmaceutically acceptable salt, polymorph, solvate, or stereoisomer thereof is administered without a drug holiday while the one or more therapeutic agents are administered with a drug holiday. It should be appreciated that the compound of Formula (I) or a pharmaceutically acceptable salt, polymorph, solvate, or stereoisomer thereof and the one or more therapeutic agents can be administered using different regimens. Thus, for instance, the compound of Formula (I) or a pharmaceutically acceptable salt, polymorph, solvate, or stereoisomer thereof may be administered continuously while the one or more therapeutic agents may be administered as one dose or a defined number of multiple doses. The administration regimen of the one or more therapeutic agents may be as indicated on a label (e.g., if the therapeutic agent is a regulated drug) and/or may be modified to optimize the biological effect of the one or more therapeutic agents and/or the biological effect of the combination of the one or more therapeutic agents and the compound of Formula (I) or a pharmaceutically acceptable salt, polymorph, solvate, or stereoisomer thereof.

In one aspect, a compound of Formula (I) (e.g., EPZ-5676 or EPZ-4777) or a pharmaceutically acceptable salt, polymorph, solvate, or stereoisomer thereof and one or more therapeutic agents are administered sequentially (either compound first or agent first). It should be appreciated that the compound of Formula (I) or a pharmaceutically acceptable salt, polymorph, solvate, or stereoisomer thereof may be administered according to any of the methods described herein, such as by continuous administration, and/or administration without a drug holiday, prior to or after the administration of the one or more therapeutic agents. As also described above, a subject may be sensitized or primed by the administration of the compound of Formula (I) or a pharmaceutically acceptable salt, polymorph, solvate, or stereoisomer thereof by any of the administration regimes described herein such as by continuous administration, and/or administration without a drug holiday, prior to the administration of the one or more therapeutic agents. Alternatively, a subject may be sensitized or primed by the administration of one or more therapeutic agents. In some embodiments, the compound of Formula (I) or a pharmaceutically acceptable salt, polymorph, solvate, or stereoisomer thereof is administered with continuous administration, and/or administration without a drug holiday and the one or more therapeutic agents are administered one or more days after or prior to the administration of the compound of Formula (I) or a pharmaceutically acceptable salt, polymorph, solvate, or stereoisomer thereof.

In some embodiments, the compound of Formula (I) or a pharmaceutically acceptable salt, polymorph, solvate, or stereoisomer thereof is administered with continuous administration, and/or administration without a drug holiday until a desirable biological effect is achieved (e.g., altered chromatin state, reduction of H3K79 methyl mark, and/or cell differentiation) prior to administration of the one or more therapeutic agents.

In some embodiments, one or more therapeutic agents are administered as indicated on label until a desirable biological effect is achieved (e.g., altered chromatin state, reduction of H3K79 methyl mark, and/or cell differentiation) prior to administration of the compound of Formula (I) or a pharmaceutically acceptable salt, polymorph, solvate, or stereoisomer thereof or the composition comprising a compound of Formula (I) (e.g., EPZ-5676 or EPZ-4777) or a pharmaceutically acceptable salt, polymorph, solvate, or stereoisomer thereof and one or more therapeutic agents.

In some embodiments, a subject is evaluated after one treatment regimen described herein for any biological effects. In some embodiments, no further treatment is required if the evaluated biological effect has reached a certain predetermined level or activity. In some embodiments, the biological effect is maturation or induction of blast cells, apoptosis of leukemic blast cells, resolution of fever, cachexia or leukemia cutis, restoration of normal haemoatopoiesis, and/or complete remission. In some embodiments, the biological effect is a durable altered chromatin state. In some embodiments, the durable altered chromatin state is decreased histone methylation. In some embodiments the decreased chromatin methylation is decreased methylation of H3K79. In some embodiments, the durable altered chromatin state is present at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the treatment.

“Combination therapy” is intended to embrace administration of these therapeutic agents in a sequential manner, wherein each therapeutic agent is administered at a different time, as well as administration of these therapeutic agents, or at least two of the therapeutic agents concurrently, or in a substantially simultaneous manner. Simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. Therapeutic agents may also be administered in alternation.

The combination therapies featured in the disclosure can result in a synergistic effect in the treatment of a disease or cancer. A “synergistic effect” is defined as where the efficacy of a combination of therapeutic agents is greater than the sum of the effects of any of the agents given alone. A synergistic effect may also be an effect that cannot be achieved by administration of any of the compounds or other therapeutic agents as single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

As provided herein, the administration of the combination of a compound of Formula (I) (e.g., EPZ-5676 or EPZ-4777) and one or more therapeutic agents provides synergistic effects. As provided herein, the combination of a compound of Formula (I) (e.g., EPZ-5676 or EPZ-4777) and therapeutic agents result in a synergistic antiproliferative response, a synergistic induction of apoptosis in leukemic cells and a synergistic induction of differentiation of leukemic cells. As provided herein synergistic effects also result when leukemic cells are sensitized by the administration of a compound of Formula (I) (e.g., EPZ-5676 or EPZ-4777) prior to the administration of therapeutic agents.

“Combination therapy” also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In another aspect, a composition of the disclosure may be administered in combination with radiation therapy. Radiation therapy can also be administered in combination with a composition of the disclosure and another chemotherapeutic agent described herein as part of a multiple agent therapy.

The disclosure also provides pharmaceutical compositions comprising a compound of Formula (I) (e.g., EPZ-5676 or EPZ-4777) or pharmaceutically acceptable salts thereof, and one or more other therapeutic agent disclosed herein, mixed with pharmaceutically suitable carriers or excipient(s) at doses to treat or prevent a disease or condition as described herein.

In one aspect, the disclosure also provides pharmaceutical compositions comprising any compound of Compound A2 and Compound D16 or pharmaceutically acceptable salts thereof, and one or more therapeutic agents, mixed with pharmaceutically suitable carriers or excipient(s) at doses to treat or prevent a disease or condition as described herein.

In another aspect, the disclosure also provides pharmaceutical compositions comprising Compound A2 (also known as EPZ-5676) which has the formula:

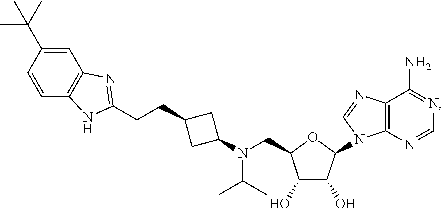 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, polymorph, solvate, or stereoisomer thereof, and one or more therapeutic agents, mixed with pharmaceutically suitable carriers or excipient(s) at doses to treat or prevent a disease or condition as described herein.

In another aspect, the disclosure also provides pharmaceutical compositions comprising Compound D16 (also known as Compound T and EPZ-4777) which has the formula:

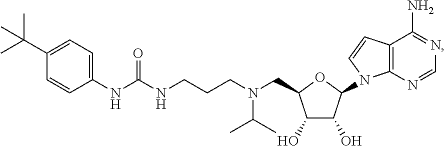 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or a pharmaceutically acceptable salt, polymorph, solvate, or stereoisomer thereof, and one or more therapeutic agents, mixed with pharmaceutically suitable carriers or excipient(s) at doses to treat or prevent a disease or condition as described herein.

The pharmaceutical compositions of the disclosure can also be administered in combination with other therapeutic agents or therapeutic modalities simultaneously, sequentially, or in alternation.

Mixtures of compositions of the disclosure can also be administered to the patient as a simple mixture or in suitable formulated pharmaceutical compositions.

A “pharmaceutical composition” is a formulation containing the compounds of the disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the disclosure can be administered to a subject in many of the well-known methods currently used for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug interaction(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

In some embodiments, the compound of Formula (I) or a pharmaceutically acceptable salt, polymorph, solvate, or stereoisomer thereof is administered continuously for at least 7, 14, 21, 28, 35, 42, 47, 56, or 64 days. In some embodiments, the compound of Formula (I) or a pharmaceutically acceptable salt, polymorph, solvate, or stereoisomer thereof is administered continuously for at least 7, 14, 21, 28, 35, 42, 47, 56, or 64 days without a drug holiday.

In some embodiments, the compound of Formula (I) or a pharmaceutically acceptable salt, polymorph, solvate, or stereoisomer thereof is administered at a dose of at least 36, 45, 54, 70, 80, or 90 mg/m²/day. In some embodiments, the compound of Formula (I) or a pharmaceutically acceptable salt, polymorph, solvate, or stereoisomer thereof is administered at a dose of at least 36, 45, 54, 70, 80, or 90 mg/m²/day continuously for at least 7, 14, 21, 28, 35, 42, 47, 56, or 64 days. In some embodiments, the compound of Formula (I) or a pharmaceutically acceptable salt, polymorph, solvate, or stereoisomer thereof is administered at a dose of at least 36, 45, 54, 70, 80, or 90 mg/m²/day continuously without a drug holiday. In some embodiments, the compound of Formula (I) or a pharmaceutically acceptable salt, polymorph, solvate, or stereoisomer thereof is administered at a dose of at least 36, 45, 54, 70, 80, or 90 mg/m²/day continuously for at least 7, 14, 21, 28, 35, 42, 47, 56, or 64 days without a drug holiday.

In some embodiments, the compound of Formula (I) or a pharmaceutically acceptable salt, polymorph, solvate, or stereoisomer thereof is administered continuously for at least 7, 14, 21, 28, 35, 42, 47, 56, or 64 days in combination with one or more therapeutic agents. In some embodiments, the compound of Formula (I) or a pharmaceutically acceptable salt, polymorph, solvate, or stereoisomer thereof is administered continuously for at least 7, 14, 21, 28, 35, 42, 47, 56, or 64 days without a drug holiday in combination with one or more therapeutic agents.

In some embodiments, the compound of Formula (I) or a pharmaceutically acceptable salt, polymorph, solvate, or stereoisomer thereof is administered at a dose of at least 36, 45, 54, 70, 80, or 90 mg/m²/day in combination with one or more therapeutic agents. In some embodiments, the compound of Formula (I) or a pharmaceutically acceptable salt, polymorph, solvate, or stereoisomer thereof is administered at a dose of at least 36, 45, 54, 70, 80, or 90 mg/m²/day continuously for at least 7, 14, 21, 28, 35, 42, 47, 56, or 64 days in combination with the one or more therapeutic agents. In some embodiments, the compound of Formula (I) or a pharmaceutically acceptable salt, polymorph, solvate, or stereoisomer thereof is administered at a dose of at least 36, 45, 54, 70, 80, or 90 mg/m²/day continuously without a drug holiday in combination with one or more therapeutic agents. In some embodiments, the compound of Formula (I) or a pharmaceutically acceptable salt, polymorph, solvate, or stereoisomer thereof is administered at a dose of at least 36, 45, 54, 70, 80, or 90 mg/m²/day continuously for at least 7, 14, 21, 28, 35, 42, 47, 56, or 64 days without a drug holiday in combination with one or more therapeutic agents.

The pharmaceutical compositions can be included in a container, pack, or dispenser together with instructions for administration.

The compounds of the disclosure are capable of further forming salts. All of these forms are also contemplated within the scope of the claimed invention.

As used herein, “pharmaceutically acceptable salts” refer to derivatives of the compounds of the disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The compounds of the disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds of the disclosure can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the disclosure can be delivered in prodrug form. Thus, the disclosure is intended to cover prodrugs of the presently disclosed compounds, methods of delivering the same and compositions containing the same. “Prodrugs” are intended to include any covalently bonded carriers that release an active parent drug of the disclosure in vivo when such prodrug is administered to a subject. Prodrugs in the disclosure are prepared by modifying functional groups present in the compound in such a way that the modifications are cleaved, either in routine manipulation or in vivo, to the parent compound. Prodrugs include compounds of the disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p1-92, Elesevier, N.Y.-Oxford (1985).

The compounds, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds can be found in Remington: the Science and Practice of Pharmacy, 19^{th} edition, Mack Publishing Co., Easton, Penn. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers.

Compounds described herein are assayed for modulation of activity, for example, histone methylation, modulation of cell growth and/or IC₅₀, described in the examples below. IC₅₀ values for DOT1L inhibition for select DOT1L inhibitors were determined as described in Example 1 and are listed below.

| | DOT1L |
|---|---|
| | IC₅₀ |
| Compound | (μM) |
| A2 | 0.00074 |

Diseases such as cancers and neurological disease can be treated by administration of modulators of protein (e.g., histone) methylation, e.g., modulators of histone methyltransferase, or histone demethylase enzyme activity. Histone methylation has been reported to be involved in aberrant expression of certain genes in cancers, and in silencing of neuronal genes in non-neuronal cells. The composition of this disclosure, e.g. a composition comprising any compound of Formula (I) or a pharmaceutically acceptable salt, polymorph, solvate, or stereoisomer thereof and one or more therapeutic agents described herein can be used to treat such diseases, i.e., to decrease or inhibit methylation of histones in affected cells or restore methylation to roughly its level in counterpart normal cells.

The disclosure provides compositions and methods for treating or alleviating a symptom of conditions and diseases the course of which can be influenced by modulating the methylation status of histones or other proteins, wherein said methylation status is mediated at least in part by the activity of DOT1L. Modulation of the methylation status of histones can in turn influence the level of expression of target genes activated by methylation, and/or target genes suppressed by methylation. The method includes administering to a subject in need of such treatment, a therapeutically effective amount of a composition of the disclosure or a pharmaceutically acceptable salt, polymorph or solvate thereof, to a subject in need of such treatment.

Modulators of methylation can be used for modulating cell proliferation, generally. For example, in some cases excessive proliferation may be reduced with agents that decrease methylation, whereas insufficient proliferation may be stimulated with agents that increase methylation. Accordingly, diseases that may be treated include hyperproliferative diseases, such as benign cell growth and malignant cell growth (cancer).

The disorder in which DOT1L-mediated protein methylation plays a part can be cancer, a cell proliferative disorder, or a precancerous condition. Exemplary cancers that may be treated include brain and CNS cancer, kidney cancer, ovarian cancer, pancreatic cancer, lung cancer, breast cancer, colon cancer, prostate cancer, or a hematological cancer. For example, the hematological cancer is leukemia or lymphoma. Preferably the cancer is leukemia. The leukemia can be acute or chronic leukemia. In some embodiments, the leukemia is acute myeloid leukemia or acute lymphocytic leukemia. In some embodiments, leukemia that may be treated is leukemia characterized by a chromosomal rearrangement on chromosome 11q23, including chimeric fusion of mixed lineage leukemia gene (MLL) or partial tandem duplication of MLL (MLL-PTD). In some embodiments, leukemia that may be treated is leukemia characterized by the presence of a genetic lesion of MLL. Such genetic lesions include chromosomal rearrangements, such as translocations, deletions, and/or duplications of the MLL gene. MLL has been categorized or characterized as having a chimeric fusion of MLL, partial tandem duplication of the MLL gene (MLL-PTD), or non-rearranged MLL.

The disorder that can be treated by the combination therapy described herein can be a disorder medicated by translocation, deletion and/or duplication of a gene on chromosome 11q23.

In general, compounds that are methylation modulators can be used for modulating cell proliferation. For example, in some cases excessive proliferation may be reduced with agents that decrease methylation, whereas insufficient proliferation may be stimulated with agents that increase methylation. Accordingly, diseases that may be treated by the compounds of the disclosure include hyperproliferative diseases, such as benign cell growth and malignant cell growth.

As used herein, a “subject in need thereof” is a subject having a disorder in which DOT1L-mediated protein methylation plays a part, or a subject having an increased risk of developing such disorder relative to the population at large. A subject in need thereof can have a precancerous condition. Preferably, a subject in need thereof has cancer. A “subject” includes a mammal. The mammal can be e.g., any mammal, e.g., a human, primate, bird, mouse, rat, fowl, dog, cat, cow, horse, goat, camel, sheep or pig. Preferably, the mammal is a human.

In some embodiments, the subject is child. In some embodiments, the subject is younger than 18 years of age. In some embodiments, the subject is younger than 18, 17, 16, 15, 14, 13, 12, 11, 10, 9, 8, 7, 6, 5, 4, 3, 2, or 1 year of age. In some embodiments, the subject is between 3 months and 18 years of age.

In some embodiments, the subject has a mutation in the RAS-RAF-MEK-ERK pathway (e.g., one or more mutations in RAS, one or more mutations RAF, one or more mutations in MEK, and/or one or more mutations in ERK). For example, the subject has a Ras mutation (e.g., H-Ras or HRAS mutation, K-Ras or KRAS mutation, or N-Ras or NRAS mutation). In some embodiments, the KRAS mutation is at A146. In some embodiments, the KRAS mutation is KRAS A146T. In some embodiments, the KRAS mutation is heterozygous. In some embodiments, the KRAS mutation is heterozygous KRAS A146T or KRAS A146T (het). In some embodiments, the KRAS mutation is at K117. In some embodiments, the KRAS mutation is KRAS K117N. In some embodiments, the KRAS mutation is homozygous. In some embodiments, the KRAS mutation is homozygous KRAS K117N or KRAS K117N (homo). In some embodiments, the NRAS mutation is at Q61. In some embodiments, the NRAS mutation is NRAS Q61R. In some embodiments, the NRAS mutation is heterozygous. In some embodiments, the NRAS mutation is heterozygous NRAS Q61R or NRAS Q61R (het). In some embodiments, the NRAS mutation is at G12. In some embodiments, the NRAS mutation is NRAS G12D. In some embodiments, the NRAS mutation is homozygous. In some embodiments, the NRAS mutation is heterozygous.

In some embodiments, the subject has an activating mutation in the RAS-RAF-MEK-ERK pathway (e.g., one or more activating mutations in RAS, one or more activating mutations RAF, one or more activating mutations in MEK, and/or one or more activating mutations in ERK). In some embodiments, the mutation in the RAS-RAF-MEK-ERK pathway results in an upregulation of the RAS-RAF-MEK-ERK pathway.

Activating Ras mutations are frequently found in many types of cancer. Activating mutations in three Ras isoforms, K-Ras, H-Ras, and N-Ras have been previously described. Activating Ras mutations are often present at codons 12, 13, or 61. See Prior et al., Cancer Res. 2012, 72(10:2457-2467), the content of which is incorporated herein by reference in its entirety. These mutations at codons 12, 13, or 61 are found among the three Ras isoforms. While these mutations are found in the Ras isoforms, certain mutated Ras isoforms are more frequently found in certain kinds of cancers. For example, mutated K-Ras is present in approximately 60% of pancreatic cancers, while hematopoietic tumors more frequently have N-Ras mutations in comparison to K-Ras mutations. According to the data present in the Catalogue of Somatic Mutations in Cancer (COSMIC) v52 Release, activating mutations of K-Ras is the most frequently found Ras isoform present in tumors (22%), followed by N-Ras (8%), and H-Ras (3%). Ibid.

Activating Ras mutations at codons 12, 13, and 61 occur more frequently in certain Ras isoforms. For example, approximately 80% of activating K-Ras mutations found in tumors occur at codon 12, whereas approximately 35% of N-Ras mutations found in tumors occur at codon 12. H-Ras activating mutations found in tumors occur approximately 50% and 40% at codons 12 and 61, respectively. See Prior et al., Cancer Res. 2012, 72(10:2457-2467). These data provide support for unique roles of mutations at codons 12, 13, and 61 in the Ras isoforms present in various cancers. Certain kinds of point mutations within codons 12, 13, and 61 are present more often in certain Ras isoforms. For example, 43% of the K-Ras mutations found in tumors had a G12D or G13D mutation, whereas tumors having an H-Ras activating mutation more frequently had a G12V mutation.

Mutations affecting the Ras-Raf-MEK-ERK pathway, including point mutations, gene deletions, and chromosomal translocations are frequently found in childhood acute lymphoblastic leukemia (ALL). See Knight and Irving, Frontiers in Oncology, 2014, 4:160, pages 1-12, the content of which is incorporated herein by reference in its entirety. Biological samples from ALL patients have indicated that many ALL cancers are associated with both a MLL H3K4 histone methyltransferase gene rearrangement and mutations affecting the RAS pathway. See Grossman et al. Leukemia, 2013, 27(9):1933-1936, the content of which are incorporated herein by reference in its entirety.

Specific cell lines have been isolated having MLL gene rearrangements which are useful to study the effect of various compounds on cancer phenotype. These cell lines include the MOLM-13 (MLL-AF9) (see Matsuo et al, Leukemia, 1997, 11, 1469-1477), OCI-AML-4 (MLL-ENL) (see Koistinen et al, Leukemia, 1991, 5(8): 704-711), THP-1 (MLL-AF9) (see Odero et al, Genes Chromosomes Cancer, 2000, 29(4):333-338, ML-2 (MLL-AF6) (see Deshpande et al, Blood, 2013, 121(13):2533-2541), and RS4-11 (MLL-AF4) (see Xia et al, PNAS, 2005, 102(39):14028-33), the contents of each of which are incorporated herein in their entireties.

The subject of the disclosure includes any human subject who has been diagnosed with, has symptoms of, or is at risk of developing a cancer or a precancerous condition.

A subject in need thereof may be a subject having a disorder associated DOT1L. A subject in need thereof can have a precancerous condition. Preferably, a subject in need thereof has cancer. A subject in need thereof can have cancer associated with DOT1L. In a preferred aspect, a subject in need thereof has one or more cancers selected from the group consisting of brain and central nervous system (CNS) cancer, head and neck cancer, kidney cancer, ovarian cancer, pancreatic cancer, leukemia, lung cancer, lymphoma, myeloma, sarcoma, breast cancer, prostate cancer and a hematological cancer. Preferably, a subject in need thereof has a hematologic cancer, wherein the hematologic cancer is leukemia or lymphoma. Exemplary leukemia is MLL. Other hematologic cancers of the disclosure can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A subject in need thereof can be one who has been previously diagnosed or identified as having cancer or a precancerous condition. A subject in need thereof can also be one who is having (suffering from) cancer or a precancerous condition. Alternatively, a subject in need thereof can be one who is having an increased risk of developing such disorder relative to the population at large (i.e., a subject who is predisposed to developing such disorder relative to the population at large).

A subject in need thereof can have cancer associated with increased expression (mRNA or protein) and/or activity level of at least one protein selected from the group consisting of HOXA9, FLT3, MEIS1, MEIS2, TBP, BCL, and DOT1L. A subject in need thereof may have increased mRNA, protein, and/or activity level of at least of at least one signaling component downstream of at least one protein selected from the group consisting of HOXA9, FLT3, MEIS1, MEIS2, TBP, BCL, and DOT1L. Such downstream components are readily known in the art, and can include other transcription factors, or signaling proteins. As used herein, the term “increase in activity” refers to increased or a gain of function of a gene product/protein compared to the wild type. Accordingly, an increase in mRNA or protein expression and/or activity levels can be detected using any suitable method available in the art.

Optionally a subject in need thereof has already undergone, is undergoing or will undergo, at least one therapeutic intervention for the cancer or precancerous condition.

A subject in need thereof may have refractory cancer on most recent therapy. “Refractory cancer” means cancer that does not respond to treatment. The cancer may be resistant at the beginning of treatment or it may become resistant during treatment. Refractory cancer is also called resistant cancer. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy.

In some embodiments, a subject in need thereof may have a secondary cancer as a result of a previous therapy. “Secondary cancer” means cancer that arises due to or as a result from previous carcinogenic therapies, such as chemotherapy. In some embodiments, the secondary cancer is a hematologic cancer, such as leukemia.

The subject may exhibit resistance to DOT1L histone methyltransferase inhibitors or any other therapeutic agent.

The disclosure also features a method of selecting a combination therapy for a subject having leukemia. The method includes the steps of: detecting the level of HOXA9, FLT3, MEIS1, MEIS2, TBP, BCL, and/or DOT1L in a sample from the subject; and selecting, based on the presence of the increased level of HOXA9, FLT3, MEIS1, MEIS2, TBP, BCL, and/or DOT1L, a combination therapy for treating leukemia. In one embodiment, the therapy includes administering to the subject a composition of the disclosure. In one embodiment, the method further includes administrating to the subject a therapeutically effective amount of a composition of the disclosure. In one embodiment, the leukemia is characterized by partial tandem duplication of the MLL gene (MLL-PTD)n. In another embodiment, the leukemia is characterized by overexpression of HOXA9, FLT3, MEIS1, MEIS2, TBP, BCL, and/or DOT1L.

The methods and uses described herein may include steps of detecting the mRNA, protein and/or activity (function) level of HOXA9, FLT3, MEIS1, MEIS2, TBP, BCL, and/or DOT1L in a sample from a subject in need thereof prior to and/or after the administration of a composition of the disclosure (e.g., a composition comprising a compound of Formula (I) (e.g., EPZ-5676 or EPZ-4777) or pharmaceutically acceptable salts thereof, and one or more therapeutic agents) to the subject. The presence of the increased level of HOXA9, FLT3, MEIS1, MEIS2, TBP, BCL, and/or DOT1L in the tested sample indicates the subject is responsive to the combination therapy described herein.

The disclosure provides personalized medicine, treatment and/or cancer management for a subject by genetic screening of increased gene expression (mRNA or protein), and/or increased function or activity level of at least one protein selected from the group consisting of HOXA9, FLT3, MEIS1, MEIS2, TBP, BCL, and DOT1L in the subject. For example, the disclosure provides methods for treating or alleviating a symptom of cancer or a precancerous condition in a subject in need thereof by determining responsiveness of the subject to a combination therapy and when the subject is responsive to the combination therapy, administering to the subject a composition of the disclosure. The responsiveness is determined by obtaining a sample from the subject and detecting increased mRNA or protein, and/or increased activity level of at least one protein selected from the group consisting of HOXA9, FLT3, MEIS1, MEIS2, TBP, BCL, and DOT1L, and the presence of such gain of expression and/or function indicates that the subject is responsive to the composition of the disclosure. Once the responsiveness of a subject is determined, a therapeutically effective amount of a composition, for example, a composition comprising a compound of Formula (I) (e.g., EPZ-5676 or EPZ-4777) or a pharmaceutically acceptable salt, polymorph, solvate, or stereoisomer thereof, and one or more therapeutic agents, can be administered. The therapeutically effective amount of a composition can be determined by one of ordinary skill in the art.

As used herein, the term “responsiveness” is interchangeable with terms “responsive”, “sensitive”, and “sensitivity”, and it is meant that a subject is showing therapeutic responses when administered a composition of the disclosure, e.g., tumor cells or tumor tissues of the subject undergo apoptosis and/or necrosis, and/or display reduced growing, dividing, or proliferation. This term is also meant that a subject will or has a higher probability, relative to the population at large, of showing therapeutic responses when administered a composition of the disclosure, e.g., tumor cells or tumor tissues of the subject undergo apoptosis and/or necrosis, and/or display reduced growing, dividing, or proliferation.

By “sample” it means any biological sample derived from the subject, includes but is not limited to, cells, tissues samples, body fluids (including, but not limited to, mucus, blood, plasma, serum, urine, saliva, and semen), tumor cells, and tumor tissues. Preferably, the sample is selected from bone marrow, peripheral blood cells, blood, plasma and serum. Samples can be provided by the subject under treatment or testing. Alternatively samples can be obtained by the physician according to routine practice in the art.

An increase in mRNA or protein expression and/or activity levels can be detected using any suitable method available in the art. For example, an increase in activity level can be detected by measuring the biological function of a gene product, such as the histone methyltransferase activity of DOT1L (i.e., methylation of histone substrates such as H3K79 by immunoblot); transcriptional activity of HOXA9, MEIS2 or MEIS1 (i.e., expression levels of HOXA9, MEIS2 or MEIS1 target genes by RT-PCR); or phosphorylation activity of FLT3 (i.e., phosphorylation status of FLT3 targets by immunoblot or radioimmunoassay). Alternatively, a gain of function mutation can be determined by detecting any alternation in a nucleic acid sequence encoding a protein selected from the group consisting of HOXA9, FLT3, MEIS1, MEIS2, TBP, BCL, and DOT1L. For example, a nucleic acid sequence encoding HOXA9, FLT3, MEIS1, MEIS2, TBP, BCL, and/or DOT1L having a gain of function mutation can be detected by whole-genome resequencing or target region resequencing (the latter also known as targeted resequencing) using suitably selected sources of DNA and polymerase chain reaction (PCR) primers in accordance with methods well known in the art. The method typically and generally entails the steps of genomic DNA purification, PCR amplification to amplify the region of interest, cycle sequencing, sequencing reaction cleanup, capillary electrophoresis, and/or data analysis. Alternatively or in addition, the method may include the use of microarray-based targeted region genomic DNA capture and/or sequencing. Kits, reagents, and methods for selecting appropriate PCR primers and performing resequencing are commercially available, for example, from Applied Biosystems, Agilent, and NimbleGen (Roche Diagnostics GmbH). Detection of mRNA expression can be detected by methods known in the art, such as Northern blot, nucleic acid PCR, and quantitative RT-PCR. Detection of polypeptide expression (i.e., wild-type or mutant) can be carried out with any suitable immunoassay in the art, such as Western blot analysis.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the disclosure encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue.

A cell proliferative disorder includes a precancer or a precancerous condition. A cell proliferative disorder includes cancer. Preferably, the methods provided herein are used to treat or alleviate a symptom of cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute lymphocytic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. Preferably, compositions of the disclosure may be used to treat a cancer selected from the group consisting of a hematologic cancer of the disclosure or a hematologic cell proliferative disorder of the disclosure. A hematologic cancer of the disclosure can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Preferably, compositions of the disclosure may be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma,” bronchioalveolar carcinoma, giant cell carcinoma, spindle cell carcinoma, and large cell neuroendocrine carcinoma. Lung cancer can include lung neoplasms having histologic and ultrastructual heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “cell proliferative disorder of the colon” is a cell proliferative disorder involving cells of the colon. Preferably, the cell proliferative disorder of the colon is colon cancer. Preferably, compositions of the disclosure may be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A “cell proliferative disorder of the pancreas” is a cell proliferative disorder involving cells of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructual heterogeneity (e.g., mixed cell types).

A “cell proliferative disorder of the prostate” is a cell proliferative disorder involving cells of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate can include hyperplasia, metaplasia, and dysplasia of the prostate.

A “cell proliferative disorder of the skin” is a cell proliferative disorder involving cells of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A “cell proliferative disorder of the ovary” is a cell proliferative disorder involving cells of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the skin can include hyperplasia, metaplasia, and dysplasia of cells of the ovary.

A “cell proliferative disorder of the breast” is a cell proliferative disorder involving cells of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

A cell proliferative disorder of the breast can be a precancerous condition of the breast. Compositions of the disclosure may be used to treat a precancerous condition of the breast. A precancerous condition of the breast can include atypical hyperplasia of the breast, ductal carcinoma in situ (DCIS), intraductal carcinoma, lobular carcinoma in situ (LCIS), lobular neoplasia, and stage 0 or grade 0 growth or lesion of the breast (e.g., stage 0 or grade 0 breast cancer, or carcinoma in situ). A precancerous condition of the breast can be staged according to the TNM classification scheme as accepted by the American Joint Committee on Cancer (AJCC), where the primary tumor (T) has been assigned a stage of T0 or Tis; and where the regional lymph nodes (N) have been assigned a stage of N0; and where distant metastasis (M) has been assigned a stage of M0.

The cell proliferative disorder of the breast can be breast cancer. Preferably, compositions of the disclosure may be used to treat breast cancer. Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphcytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructual heterogeneity (e.g., mixed cell types).

Preferably, compound of the disclosure, or a pharmaceutically acceptable salt, polymorph, or solvate thereof, may be used to treat breast cancer. A breast cancer that is to be treated can include familial breast cancer. A breast cancer that is to be treated can include sporadic breast cancer. A breast cancer that is to be treated can arise in a male subject. A breast cancer that is to be treated can arise in a female subject. A breast cancer that is to be treated can arise in a premenopausal female subject or a postmenopausal female subject. A breast cancer that is to be treated can arise in a subject equal to or older than 30 years old, or a subject younger than 30 years old. A breast cancer that is to be treated has arisen in a subject equal to or older than 50 years old, or a subject younger than 50 years old. A breast cancer that is to be treated can arise in a subject equal to or older than 70 years old, or a subject younger than 70 years old.

A breast cancer that is to be treated can be typed to identify a familial or spontaneous mutation in BRCA1, BRCA2, or p53. A breast cancer that is to be treated can be typed as having a HER2/neu gene amplification, as overexpressing HER2/neu, or as having a low, intermediate or high level of HER2/neu expression. A breast cancer that is to be treated can be typed for a marker selected from the group consisting of estrogen receptor (ER), progesterone receptor (PR), human epidermal growth factor receptor-2, Ki-67, CA15-3, CA 27-29, and c-Met. A breast cancer that is to be treated can be typed as ER-unknown, ER-rich or ER-poor. A breast cancer that is to be treated can be typed as ER-negative or ER-positive. ER-typing of a breast cancer may be performed by any reproducible means. ER-typing of a breast cancer may be performed as set forth in Onkologie 27: 175-179 (2004). A breast cancer that is to be treated can be typed as PR-unknown, PR-rich, or PR-poor. A breast cancer that is to be treated can be typed as PR-negative or PR-positive. A breast cancer that is to be treated can be typed as receptor positive or receptor negative. A breast cancer that is to be treated can be typed as being associated with elevated blood levels of CA 15-3, or CA 27-29, or both.

A breast cancer that is to be treated can include a localized tumor of the breast. A breast cancer that is to be treated can include a tumor of the breast that is associated with a negative sentinel lymph node (SLN) biopsy. A breast cancer that is to be treated can include a tumor of the breast that is associated with a positive sentinel lymph node (SLN) biopsy. A breast cancer that is to be treated can include a tumor of the breast that is associated with one or more positive axillary lymph nodes, where the axillary lymph nodes have been staged by any applicable method. A breast cancer that is to be treated can include a tumor of the breast that has been typed as having nodal negative status (e.g., node-negative) or nodal positive status (e.g., node-positive). A breast cancer that is to be treated can include a tumor of the breast that has metastasized to other locations in the body. A breast cancer that is to be treated can be classified as having metastasized to a location selected from the group consisting of bone, lung, liver, or brain. A breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

A compound of the disclosure, or a pharmaceutically acceptable salt, polymorph or solvate thereof, may be used to treat or prevent a cell proliferative disorder of the breast, or to treat or prevent breast cancer, in a subject having an increased risk of developing breast cancer relative to the population at large. A subject with an increased risk of developing breast cancer relative to the population at large is a female subject with a family history or personal history of breast cancer. A subject with an increased risk of developing breast cancer relative to the population at large is a female subject having a germ-line or spontaneous mutation in BRCA1 or BRCA2, or both. A subject with an increased risk of developing breast cancer relative to the population at large is a female subject with a family history of breast cancer and a germ-line or spontaneous mutation in BRCA1 or BRCA2, or both. A subject with an increased risk of developing breast cancer relative to the population at large is a female who is greater than 30 years old, greater than 40 years old, greater than 50 years old, greater than 60 years old, greater than 70 years old, greater than 80 years old, or greater than 90 years old. A subject with an increased risk of developing breast cancer relative to the population at large is a subject with atypical hyperplasia of the breast, ductal carcinoma in situ (DCIS), intraductal carcinoma, lobular carcinoma in situ (LCIS), lobular neoplasia, or a stage 0 growth or lesion of the breast (e.g., stage 0 or grade 0 breast cancer, or carcinoma in situ).

A breast cancer that is to be treated can histologically graded according to the Scarff-Bloom-Richardson system, wherein a breast tumor has been assigned a mitosis count score of 1, 2, or 3; a nuclear pleiomorphism score of 1, 2, or 3; a tubule formation score of 1, 2, or 3; and a total Scarff-Bloom-Richardson score of between 3 and 9. A breast cancer that is to be treated can be assigned a tumor grade according to the International Consensus Panel on the Treatment of Breast Cancer selected from the group consisting of grade 1, grade 1-2, grade 2, grade 2-3, or grade 3.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “candidate compound” refers to a compound of the disclosure, or a pharmaceutically acceptable salt, polymorph or solvate thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the disclosure, or a pharmaceutically acceptable salt, polymorph or solvate thereof. The biological or medical response can be the treatment of cancer. The biological or medical response can be treatment or prevention of a cell proliferative disorder. In vitro or in vivo biological assays can include, but are not limited to, enzymatic activity assays, electrophoretic mobility shift assays, reporter gene assays, in vitro cell viability assays, and the assays described herein.

For example, an in vitro biological assay that can be used includes the steps of (1) mixing a histone substrate (e.g., an isolated histone sample for a histone or modified histone of interest, or an isolated oligonucleosome substrate) with recombinant DOT1L enzyme (e.g., recombinant protein containing amino acids 1-416); (2) adding a candidate compound of the disclosure to this mixture; (3) adding non-radioactive and ³H-labeled S-Adenosyl methionine (SAM) to start the reaction; (4) adding excessive amount of non-radioactive SAM to stop the reaction; (4) washing off the free non-incorporated ³H-SAM; and (5) detecting the quantity of ³H-labeled histone substrate by any methods known in the art (e.g., by a PerkinElmer TopCount platereader).

For example, an in vitro cell viability assay that can be used includes the steps of (1) culturing cells (e.g., EOL-1, KOPM-88, Molm13, MV411, LOUCY, SemK2, Reh, HL60, BV173, or Jurkat cells) in the presence of increasing concentration of candidate compound (e.g., Compound A2, Compound D16); (2) determining viable cell number every 3-4 days by methods known in the art (e.g., using the Millipore Guava Viacount assay); (3) plotting concentration-dependence growth curves; and optionally (4) calculating IC₅₀ values from the concentration-dependence growth curves using methods known in the art (e.g., using GraphPad Prism Software).

For example, a histone methylation assay that can be used includes the steps of (1) culturing cells (e.g., EOL-1, KOPM-88, Molm13, MV411, LOUCY, SemK2, Reh, HL60, BV173, or Jurkat cells) in the presence of candidate compound (e.g., Compound A2 or Compound D16); (2) harvesting the cells; (3) extracting histone proteins, using methods known in the art (e.g., sulfuric acid precipitation); (4) fractionating histone extracts by SDS-PAGE electrophoresis and transferring to a filter; (5) probing the filter with antibodies specific to a protein or methylated-protein of interest (e.g., H3K79me2-specific antibody and total histone H3-specific antibody); and (6) detecting the signal of the antibodies using methods known in the art (e.g., Li-cor Odyssey infrared imager).

For example, a gene expression assay that can be used includes the steps of (1) culturing cells (e.g., EOL-1, KOPM-88, Molm13, MV411, LOUCY, SemK2, Reh, HL60, BV173, or Jurkat cells) in the presence or absence of candidate compound (e.g., Compound A2 or Compound D16); (2) harvesting the cells; (3) extracting the RNA using methods known in the art (e.g., Qiagen RNeasy Kit); (4) synthesizing cDNA from the extracted RNA (e.g., Applied Biosystems reverse transcriptase kit); (5) preparing qPCR reactions using, for example, primers and probes (e.g., predesigned labeled primer and probe sets for HOXA9, FLT3, MEIS1, MEIS2, TBP, BCL, DOT1L, and β2-microglobulin from Applied Biosystems), synthesized sample cDNA, and qPCR master mix reagent (e.g., Applied Biosystems Taqman universal PCR master mix); (6) running samples on PCR machine (e.g., Applied Biosystems); (7) analysis of the data and calculation of relative gene expression.

As used herein, “monotherapy” refers to the administration of a single active or therapeutic compound to a subject in need thereof. Preferably, monotherapy will involve administration of a therapeutically effective amount of a single active compound. For example, cancer monotherapy with one of the compound of the disclosure, or a pharmaceutically acceptable salt, analog or derivative thereof, to a subject in need of treatment of cancer. In one aspect, the single active compound is a compound of the disclosure, or a pharmaceutically acceptable salt, polymorph or solvate thereof.

As used herein, “treating” or “treat” describes the management and care of a patient for the purpose of combating a disease, condition, or disorder and includes the administration of a compound of the disclosure, or a pharmaceutically acceptable salt, polymorph or solvate thereof, to alleviate the symptoms or complications of a disease, condition or disorder, or to eliminate the disease, condition or disorder.

A compound of the disclosure, or a pharmaceutically acceptable salt, polymorph or solvate thereof, can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, at the World Wide Web (www) cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, at the World Wide Web (www) cancer.gov). Furthermore, severity describes a nuclear grade, which refers to the size and shape of the nucleus in tumor cells and the percentage of tumor cells that are dividing (see, National Cancer Institute, at the World Wide Web (www) cancer.gov).

In another aspect of the invention, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

But sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage.

A cancer may also cause symptoms such as fever, fatigue, or weight loss. This may be because cancer cells use up much of the body's energy supply or release substances that change the body's metabolism. Or the cancer may cause the immune system to react in ways that produce these symptoms.

Sometimes, cancer cells release substances into the bloodstream that cause symptoms not usually thought to result from cancers. For example, some cancers of the pancreas can release substances which cause blood clots to develop in veins of the legs. Some lung cancers make hormone-like substances that affect blood calcium levels, affecting nerves and muscles and causing weakness and dizziness.

Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma.

Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease.

Along with cancers of the skin (see next section), some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the instant disclosure.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the disclosure, or a pharmaceutically acceptable salt, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the disclosure, or a pharmaceutically acceptable salt, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

As used herein, the term “selectively” means tending to occur at a higher frequency in one population than in another population. The compared populations can be cell populations. Preferably, a compound of the disclosure, or a pharmaceutically acceptable salt, polymorph or solvate thereof, acts selectively on a cancer or precancerous cell but not on a normal cell. Preferably, a compound of the disclosure, or a pharmaceutically acceptable salt, polymorph or solvate thereof, acts selectively to modulate one molecular target (e.g., a target protein methyltransferase) but does not significantly modulate another molecular target (e.g., a non-target protein methyltransferase). The disclosure also provides a method for selectively inhibiting the activity of an enzyme, such as a protein methyltransferase. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

A composition of the disclosure e.g., a composition comprising a compound of Formula (I) (e.g., EPZ-5676 or EPZ-4777) or a pharmaceutically acceptable salt, polymorph or solvate thereof and one or more therapeutic agents, can modulate the activity of a molecular target (e.g., a target protein methyltransferase). Modulating refers to stimulating or inhibiting an activity of a molecular target. Preferably, a composition of the disclosure modulates the activity of a molecular target if it stimulates or inhibits the activity of the molecular target by at least 2-fold relative to the activity of the molecular target under the same conditions but lacking only the presence of said compound. More preferably, a composition of the disclosure modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

As used herein, the term “isozyme selective” means preferential inhibition or stimulation of a first isoform of an enzyme in comparison to a second isoform of an enzyme (e.g., preferential inhibition or stimulation of a protein methyltransferase isozyme alpha in comparison to a protein methyltransferase isozyme beta). Preferably, a composition of the disclosure demonstrates a minimum of a fourfold differential, preferably a tenfold differential, more preferably a fifty fold differential, in the dosage required to achieve a biological effect. Preferably, a composition of the disclosure demonstrates this differential across the range of inhibition, and the differential is exemplified at the IC₅₀, i.e., a 50% inhibition, for a molecular target of interest.

Administering a composition of the disclosure to a cell or a subject in need thereof can result in modulation (i.e., stimulation or inhibition) of an activity of a protein methyltransferase of interest. Several intracellular targets can be modulated with the compounds of the disclosure, including, but not limited to, protein methyltransferase.

As used herein, “a cell cycle checkpoint pathway” refers to a biochemical pathway that is involved in modulation of a cell cycle checkpoint. A cell cycle checkpoint pathway may have stimulatory or inhibitory effects, or both, on one or more functions comprising a cell cycle checkpoint. A cell cycle checkpoint pathway is comprised of at least two compositions of matter, preferably proteins, both of which contribute to modulation of a cell cycle checkpoint. A cell cycle checkpoint pathway may be activated through an activation of one or more members of the cell cycle checkpoint pathway. Preferably, a cell cycle checkpoint pathway is a biochemical signaling pathway.

As used herein, “cell cycle checkpoint regulator” refers to a composition of matter that can function, at least in part, in modulation of a cell cycle checkpoint. A cell cycle checkpoint regulator may have stimulatory or inhibitory effects, or both, on one or more functions comprising a cell cycle checkpoint. A cell cycle checkpoint regulator can be a protein or not a protein.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

Preferably, an effective amount of a composition of the disclosure is not significantly cytotoxic to normal cells. A therapeutically effective amount of a composition is not significantly cytotoxic to normal cells if administration of the composition in a therapeutically effective amount does not induce cell death in greater than 10% of normal cells. A therapeutically effective amount of a composition does not significantly affect the viability of normal cells if administration of the composition in a therapeutically effective amount does not induce cell death in greater than 10% of normal cells. In an aspect, cell death occurs by apoptosis.

Contacting a cell with a composition of the disclosure can induce or activate cell death selectively in cancer cells. Administering to a subject in need thereof a composition of the disclosure can induce or activate cell death selectively in cancer cells. Contacting a cell with a composition of the disclosure can induce cell death selectively in one or more cells affected by a cell proliferative disorder. Preferably, administering to a subject in need thereof a composition of the disclosure induces cell death selectively in one or more cells affected by a cell proliferative disorder.

The disclosure relates to a method of treating or alleviating a symptom of cancer by administering a composition of the disclosure to a subject in need thereof, where administration of the composition results in one or more of the following: accumulation of cells in G1 and/or S phase of the cell cycle, cytotoxicity via cell death in cancer cells without a significant amount of cell death in normal cells, antitumor activity in animals with a therapeutic index of at least 2, and activation of a cell cycle checkpoint. As used herein, “therapeutic index” is the maximum tolerated dose divided by the efficacious dose.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Penn., 18^{th} edition (1990). These texts can, of course, also be referred to in making or using an aspect of the invention

The composition of the disclosure can also be utilized to treat or alleviate a symptom of neurologic diseases or disorders. Neurologic diseases or disorders that may be treated with the compounds of this disclosure include epilepsy, schizophrenia, bipolar disorder or other psychological and/or psychiatric disorders, neuropathies, skeletal muscle atrophy, and neurodegenerative diseases, e.g., a neurodegenerative disease. Exemplary neurodegenerative diseases include: Alzheimer's, Amyotrophic Lateral Sclerosis (ALS), and Parkinson's disease. Another class of neurodegenerative diseases includes diseases caused at least in part by aggregation of poly-glutamine. Diseases of this class include: Huntington's Diseases, Spinalbulbar Muscular Atrophy (SBMA or Kennedy's Disease) Dentatorubropallidoluysian Atrophy (DRPLA), Spinocerebellar Ataxia 1 (SCA1), Spinocerebellar Ataxia 2 (SCA2), Machado-Joseph Disease (MJD; SCA3), Spinocerebellar Ataxia 6 (SCA6), Spinocerebellar Ataxia 7 (SCAT), and Spinocerebellar Ataxia 12 (SCA12).

Any other disease in which epigenetic methylation, which is mediated by DOT1, plays a role may be treatable or preventable using compounds and methods described herein.

The disclosure provides use of a composition disclosed herein for inhibiting DOT1L activity in a cell. Still another aspect of the invention relates to a use of a composition disclosed herein for reducing the level of methylation of histone H3 lysine residue 79 (H3-K79) in a cell.

Any of the above aspects and embodiments can be combined with any other aspect or embodiment.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLE 1

### DOT1L Combination Studies in MLL-Rearranged Cell Lines Methods

The acute myelogenous leukemia cell lines MV4-11 (MLL-AF4) and MOLM-13 (MLL-AF9) were obtained from American Type Culture Collection (ATCC; Rockville, Md.) and Deutsche Sammlung von Mikroorganismen and Zellkulturen (DSMZ; Braunschweig, Germany) respectively. MV4-11 cells were maintained in IMDM (Invitrogen, supplemented with 10% heat inactivated fetal bovine serum (Life Technologies, Grand Island, N.Y.). MOLM-13 cells were maintained in RPMI-1640 supplemented with 10% fetal bovine serum (Life Technologies, Grand Island, N.Y.). Cultures were maintained in a humidified atmosphere including 5% CO₂.

Studies were performed using MLL-rearranged cell lines in vitro to evaluate the anti-proliferative effect of a combination of two agents together on cell growth. Initial proliferation studies were performed to determine the IC₅₀ of a given compound in each cell line. The cell counts were measured by ATP quantitation using the Promega Cell Titer Glo kit and luminescence values corresponded to the amount of ATP in a given well.

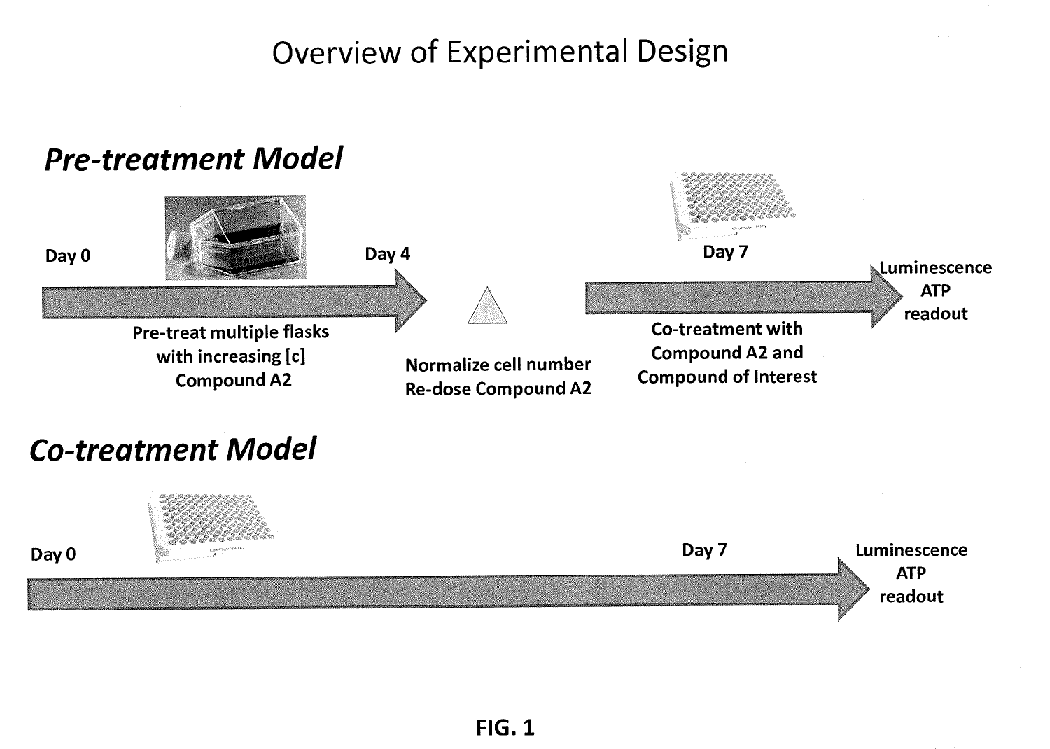

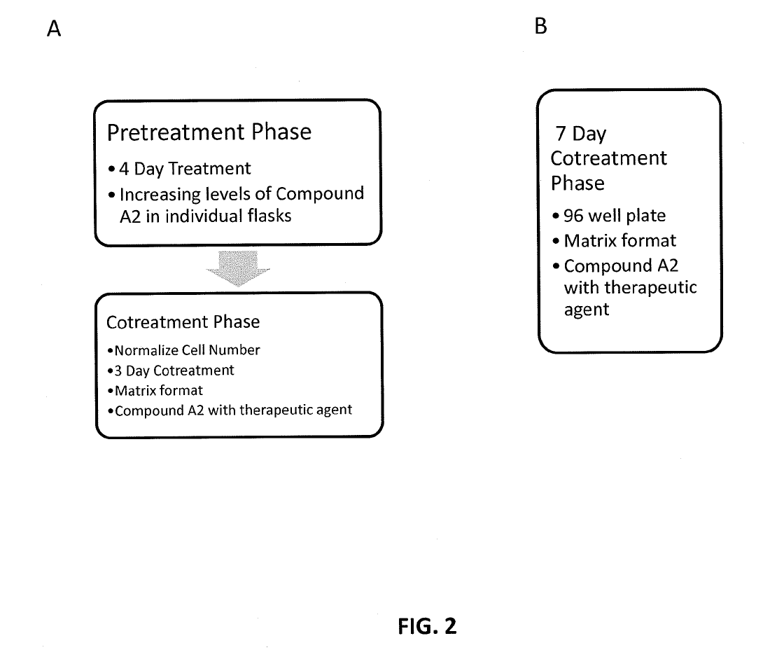

Compounds were tested in combination with Compound A2 to study their effect on cell proliferation in either a 4+3 model (cells were pretreated with increasing concentrations of Compound A2 for 4 days, followed by a co-treatment with Compound A2 with test article for 3 days) or a 7 day co-treatment model (FIGS. 1 and 2).

### Results

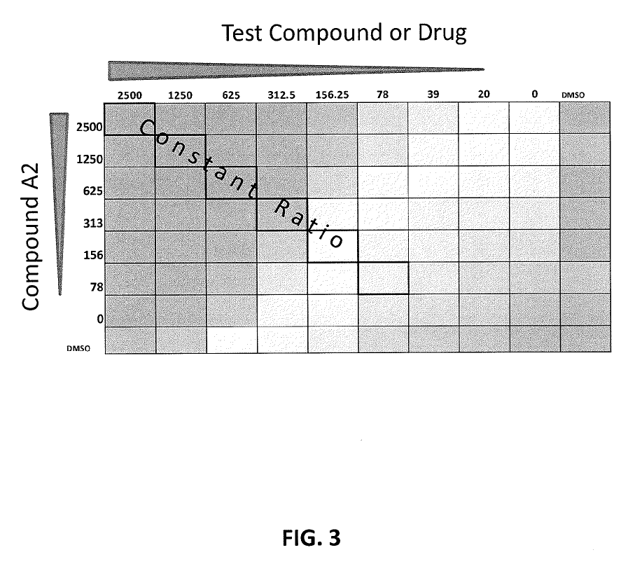

Compounds were evaluated for synergy in the co-treatment phase by testing the compounds in a concentration range which was bracketed around their IC₅₀ values. The compounds were plated to a 96 well plate in a matrix format (FIG. 3) which includes increasing concentrations of each drug in the combination in a constant ratio, in addition to the effect of each compound alone in the study. Cells were seeded and grown in the log-linear phase for 3 or 7 days in the co-treatment phase. Minimum inhibition (DMSO alone) controls were used in each plate to calculate fraction affected (Fa) of a test well. DMSO concentration was kept at 0.1% v/v.

The drug combination analysis was performed utilizing the Chou-Talalay method Synergy was determined using the software package Calcusyn by Biosoft. The combination index (CI) is a quantitative term used to describe the level of synergy or antagonism in a given test system. A combination index less than one indicates synergy, and a CI greater than one indicates antagonism. Further, strong synergism is achieved when the CI value falls below 0.3.

Pretreatment with Compound A2 followed by cotreatment with either Ara-C or Daunorubicin demonstrated synergy in both MV4-11 and MOLM-13 cell lines.

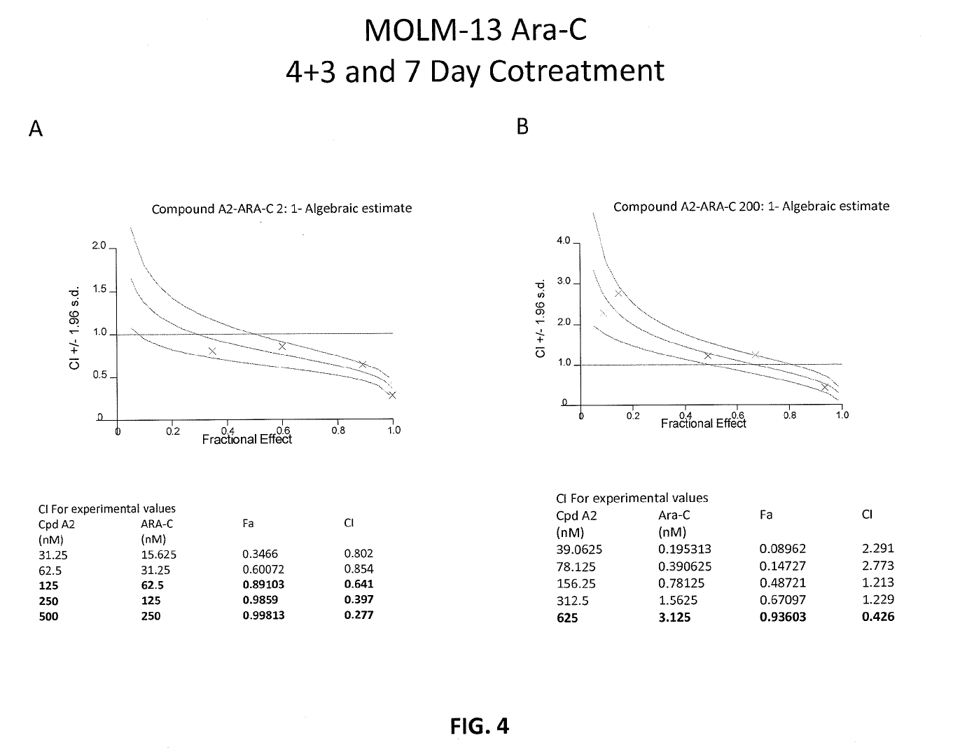

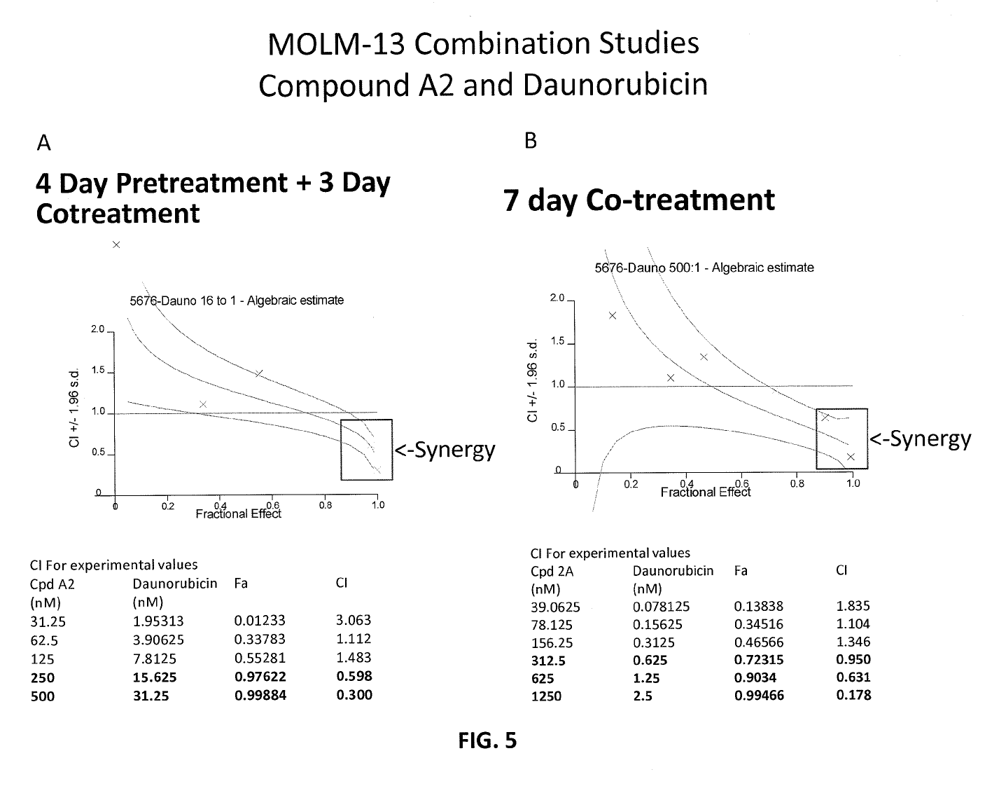

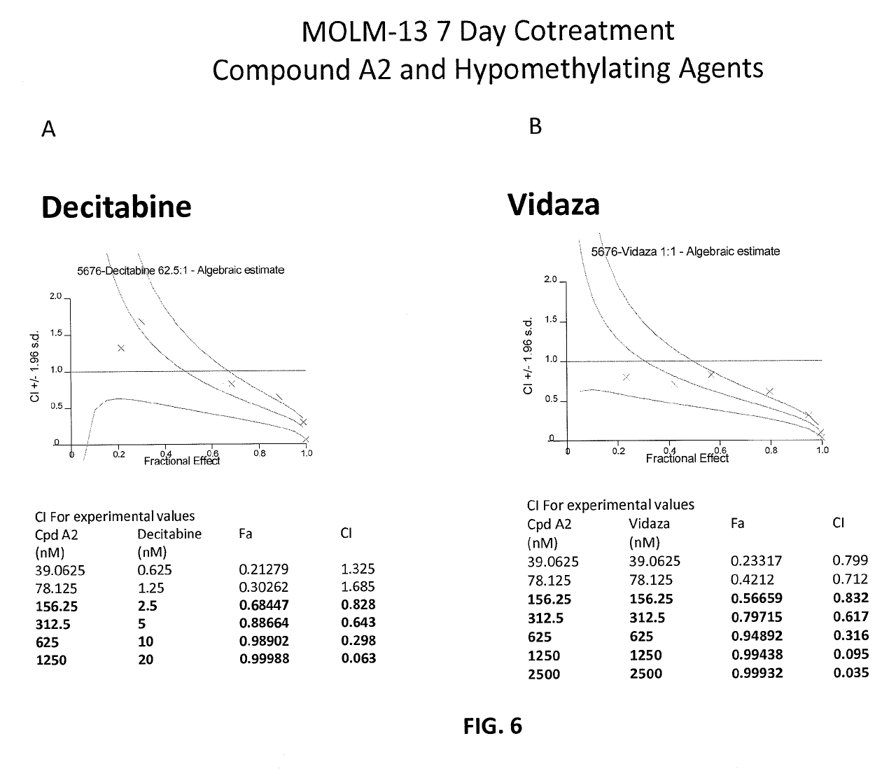

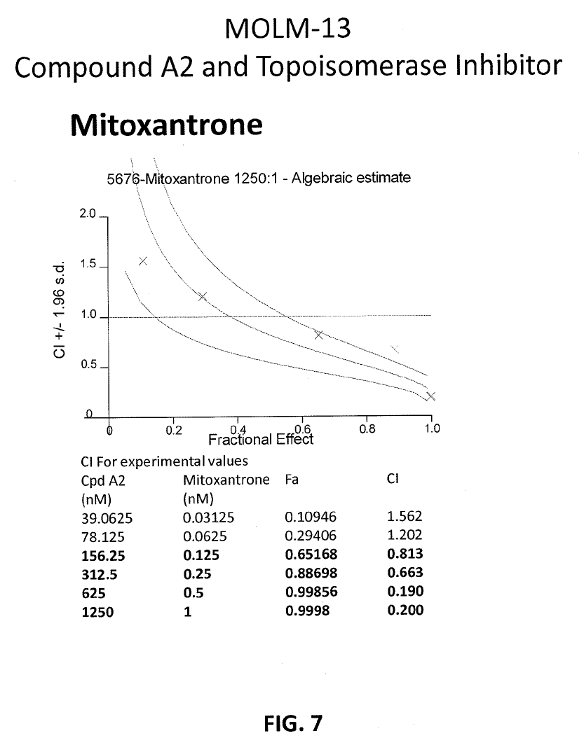

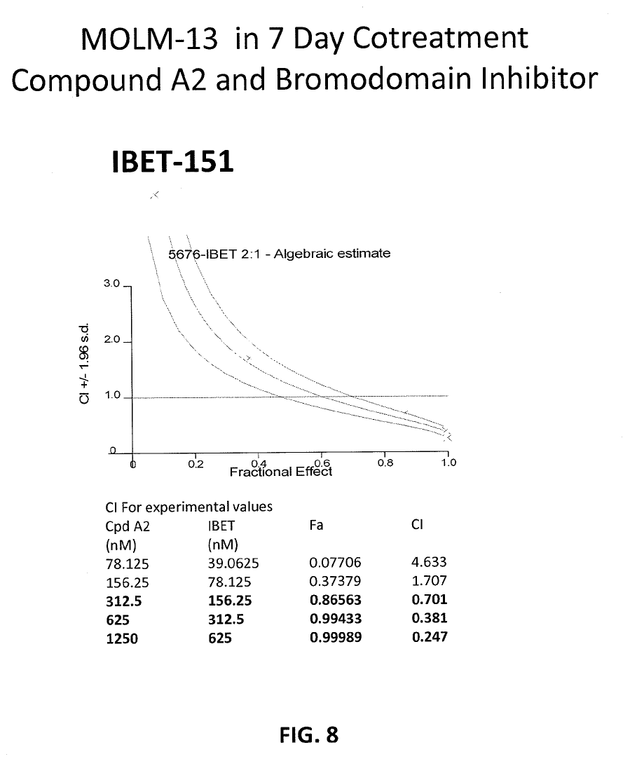

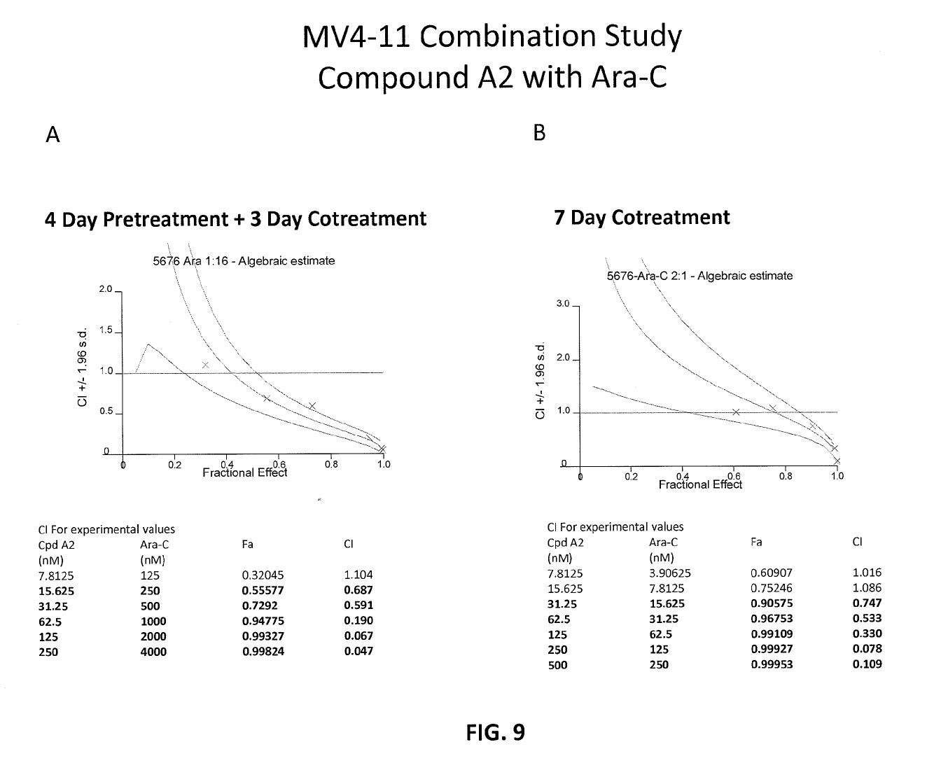

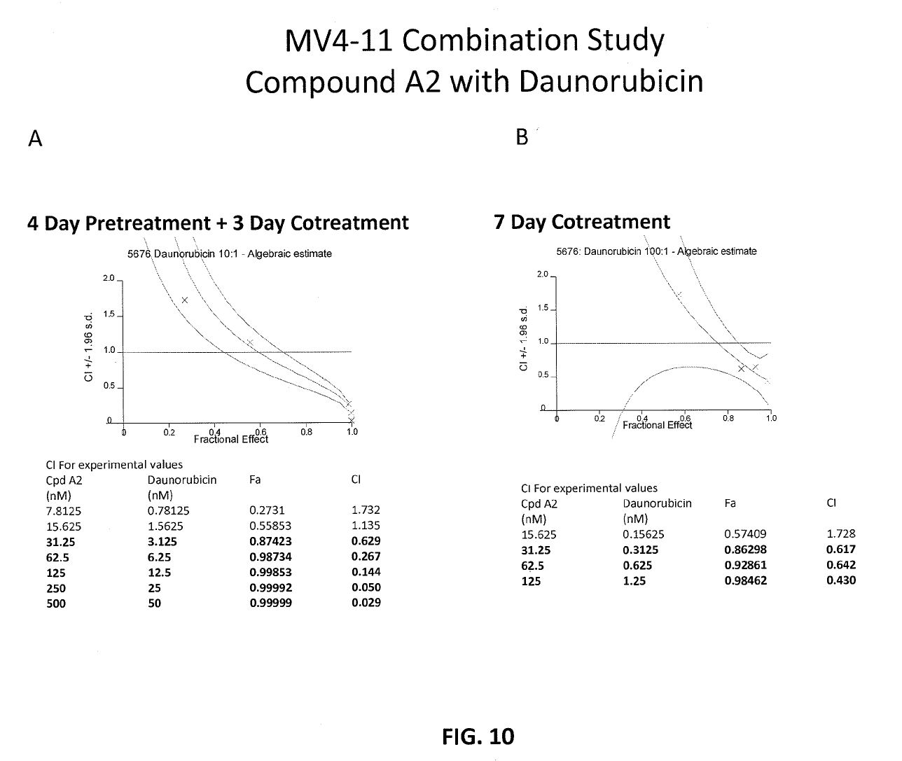

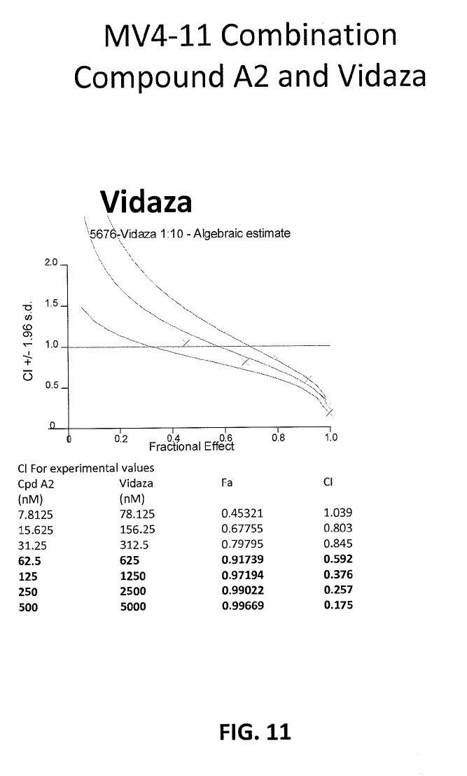

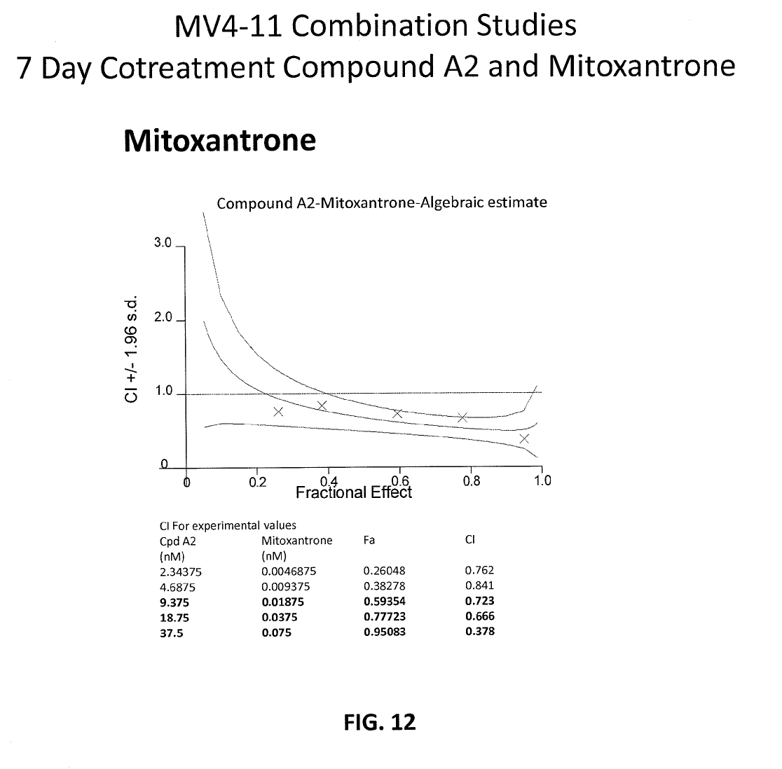

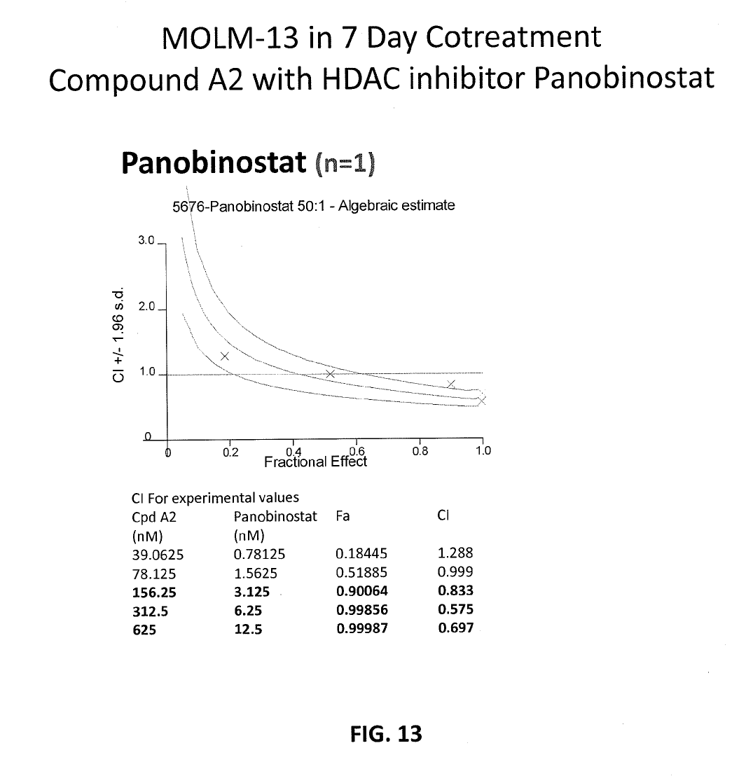

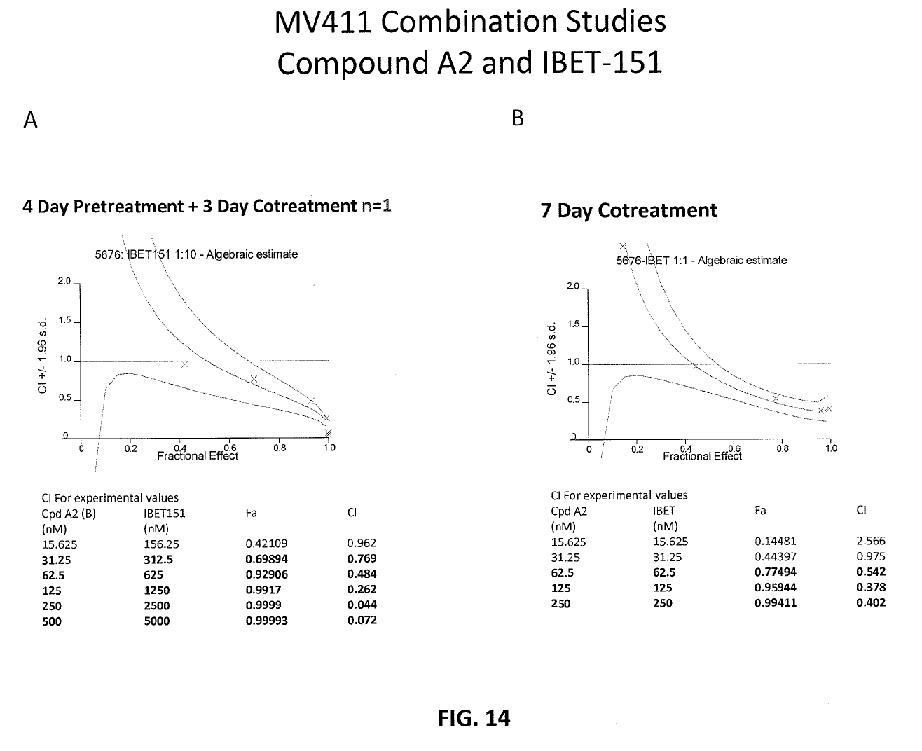

In a seven day cotreatment model, synergy with Compound A2 has been shown with the following drugs in the MOLM-13 (MLL-AF9 rearranged) cell line: Ara-C (FIG. 4), Daunorubicin (FIG. 5) Decitabine (strong) (FIG. 6), Vidaza (strong) (FIG. 6), Mitoxantrone (FIG. 7), IBET-151 (FIG. 8). Synergy with Compound A2 has been shown with the following drugs in MV4-11 (MLL-AF4) cell line: Ara-C (FIG. 9), Daunorubicin (FIG. 10), Vidaza (FIG. 11), Mitoxantrone (FIG. 12), IBET-151 (FIG. 14).

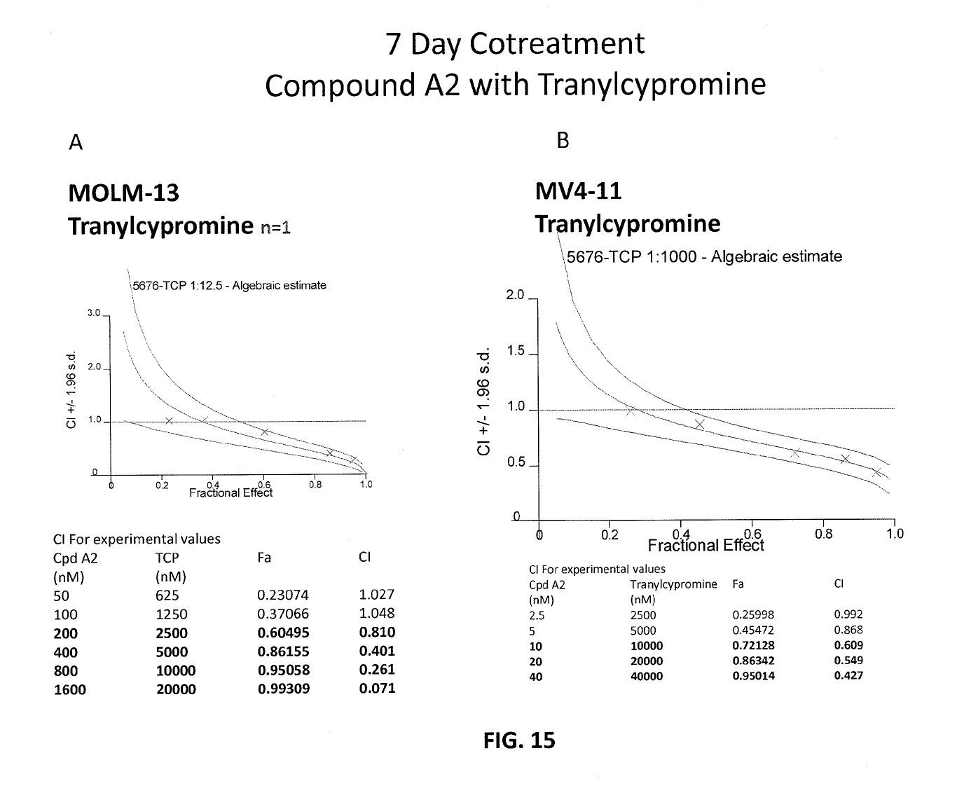

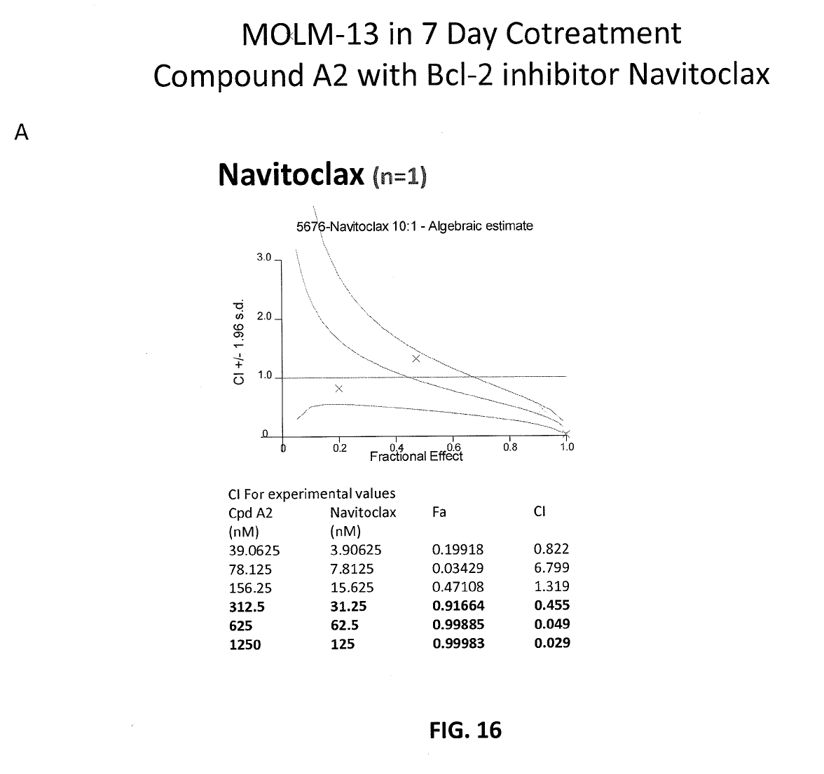

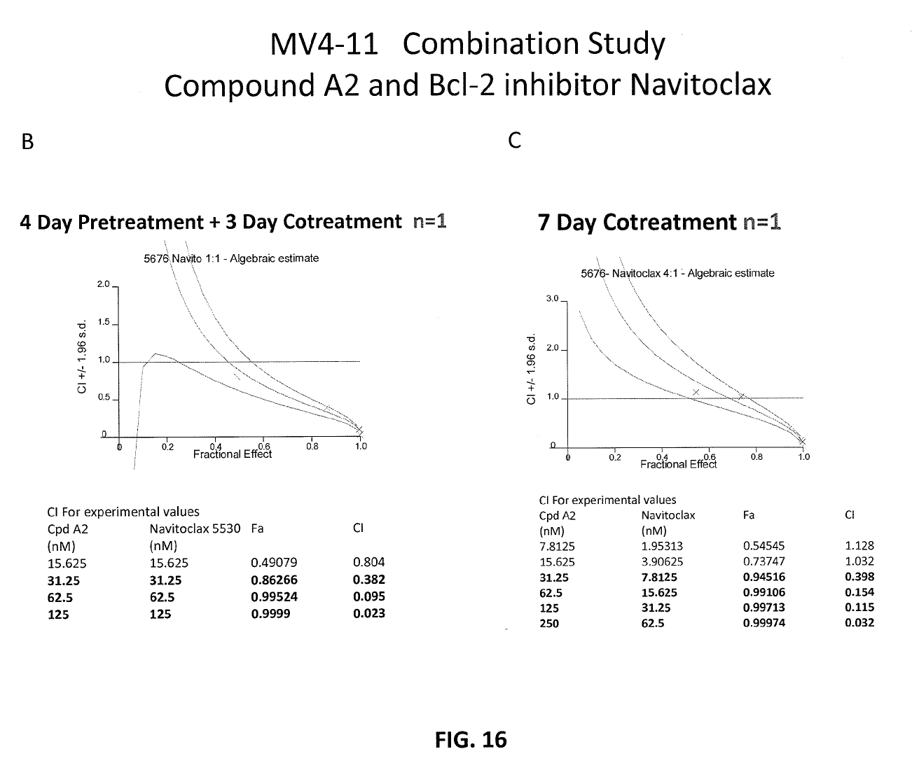

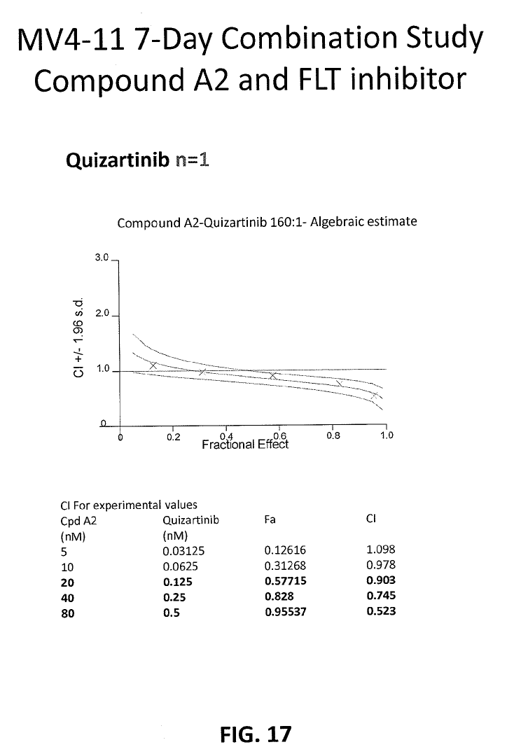

To this end, it has been demonstrated that LSD1 inhibitor, Tranylcypromine (FIG. 15) and Bcl-2 inhibitor, Navitoclax (FIG. 16) show synergy with Compound A2 in both MOLM (FIGS. 15 and 16) and MV4-11 cell lines (FIGS. 15 and 16). Quizartinib (FIG. 17), a FLT inhibitor has also shown synergy in MV4-11 cells.

TABLE 1
Summary table for combination studies of Compound A2 and
exemplary anti-cancer agents.

| | | MOLM-13 | MV4-11 |
|---|---|---|---|
| 4 + 3 Model | Ara-C | Synergy | Synergy |
| | Daunorubicin | Synergy | Synergy |
| 7 Day Cotreatment | Ara-C | Synergy | Synergy |
| | Daunorubicin | Synergy | Synergy |
| | Decitabine | Strong Synergy | Additive |
| | | | (no data shown) |
| | Vidaza | Strong Synergy | Synergy |
| | Mitoxantrone | Synergy | Synergy |
| | IBET-151 | Synergy | Synergy |

### EXAMPLE 2

### DOT1L Inhibitor Compound A2 Displays Synergistic Antiproliferative Activity in Combination with Standard of Care Drugs or DNA Hypomethylating Agents in MLL-Rearranged Leukemia Cells

The activity of Compound A2 in combination with current standard of care agents for acute leukemias as well as other chromatin modifying drugs was evaluated in cell proliferation assays with three human acute leukemia cell lines; Molm-13 (MLL-AF9 expressing acute myeloid leukemia (AML)), MV4-11 (MLL-AF4 expressing acute biphenotypic leukemia cell line) and SKM-1 (non-MLL-rearranged AML). A high density combination platform suitable for testing the antiproliferative activity of a complete titration matrix of two agents with multiple replicate points was established to enable generation of statistically meaningful results. This platform was used to evaluate the anti-proliferative effects of Compound A2 combinations tested in a co-treatment model in which the second agent was added along with Compound A2 at the beginning of the assay, or in a pre-treatment model in which cells were incubated for several days in the presence of Compound A2 prior to the addition of the second agent. The drug combination analysis was performed using the Chou-Talalay method [Chou TC Pharmacological Reviews 2006]. Graphs representing values of combination index (CI) versus Fractional effect (Fa) known as Fa-CI plots were generated and synergy was evaluated. Drug synergy was statistically defined by CI values less than 1, antagonism by CI>1 and additive effect by CI equal to 1.

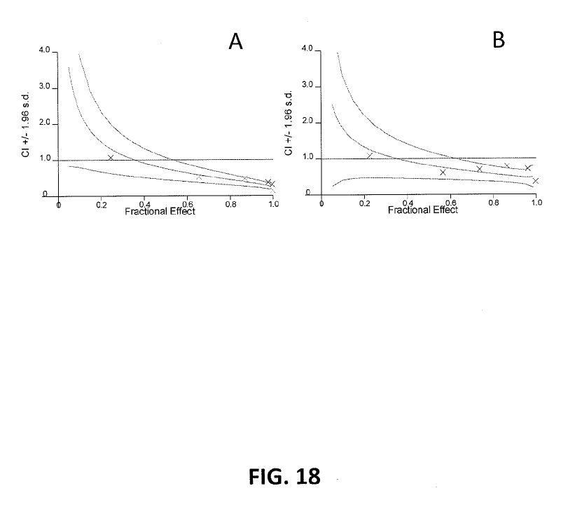

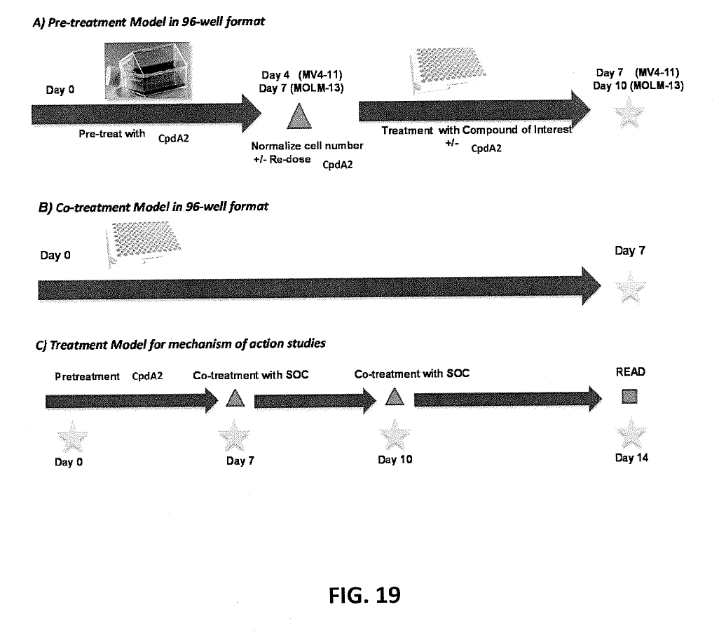

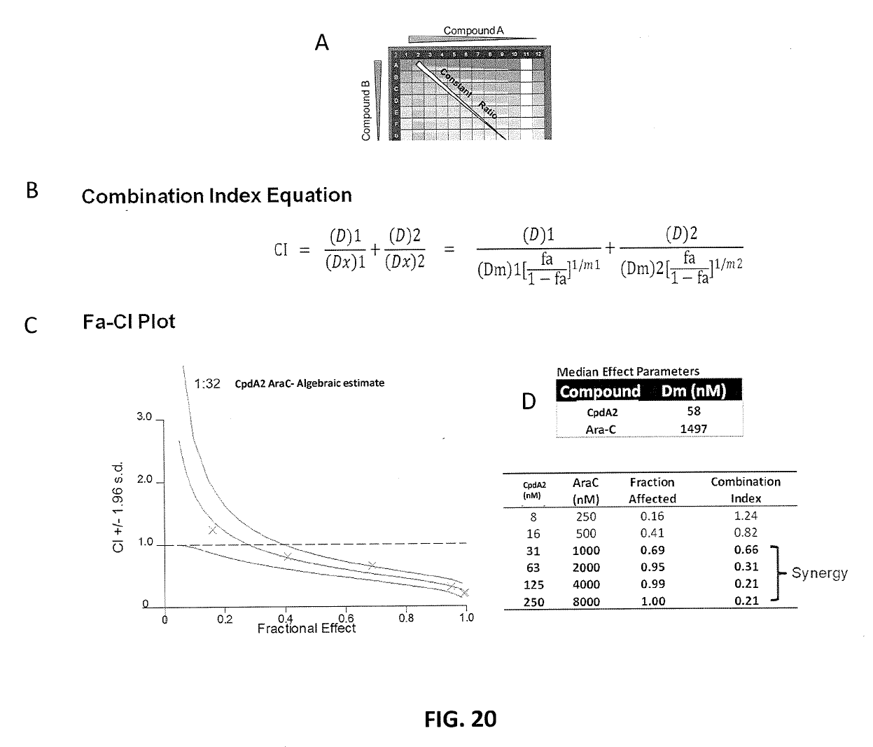

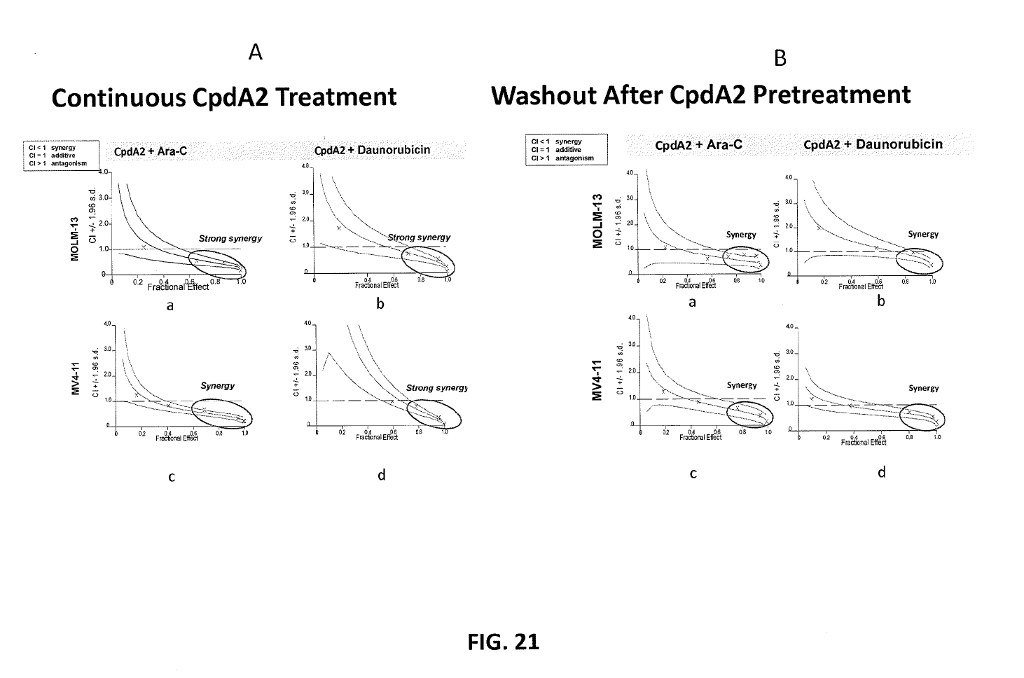

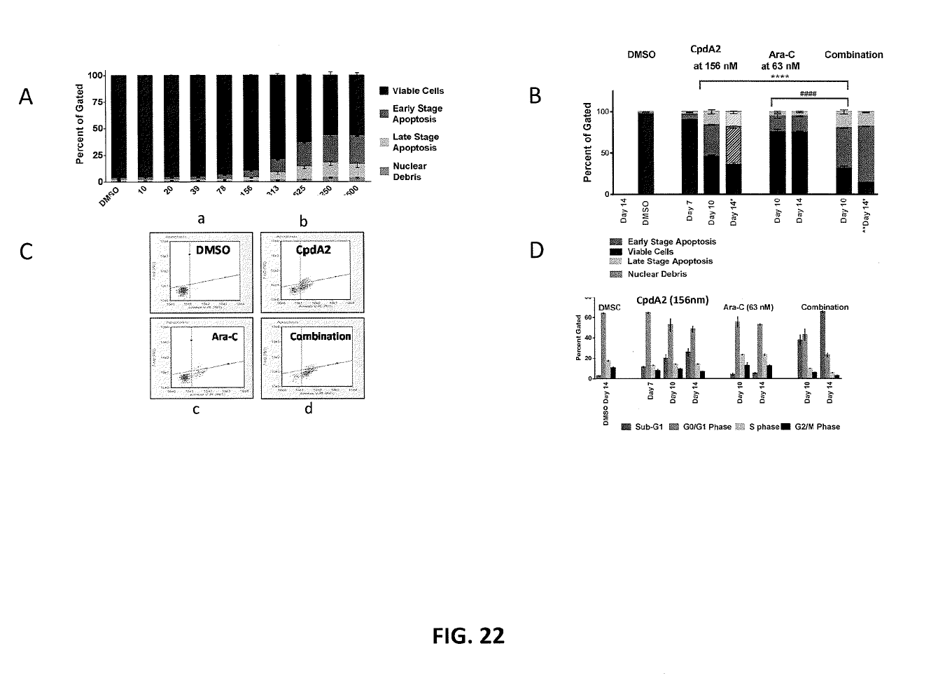

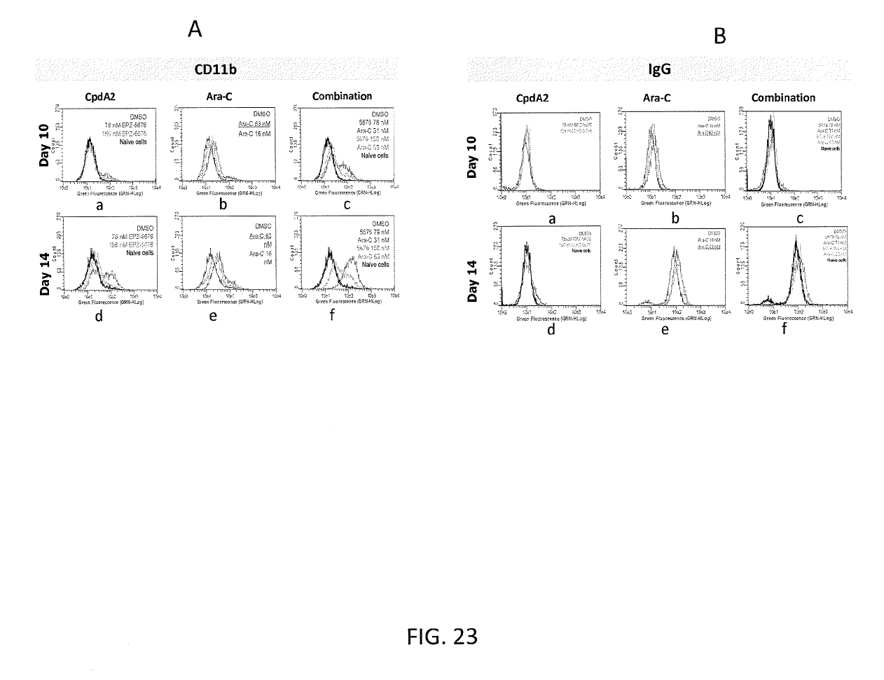

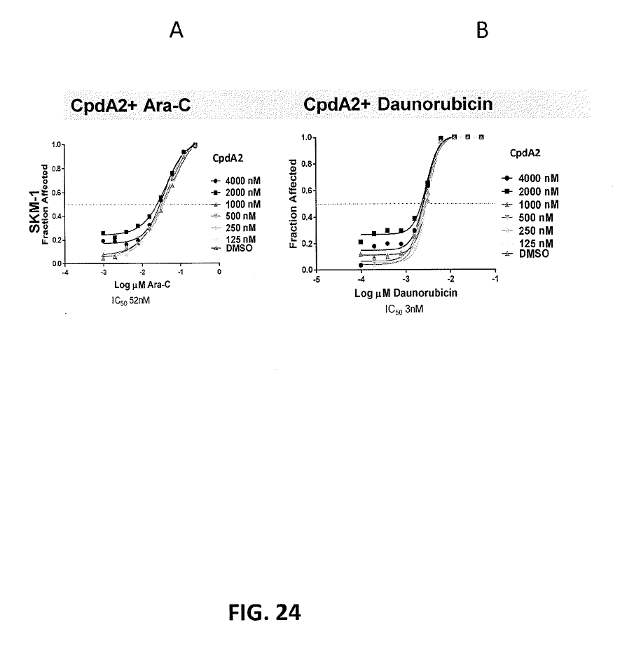

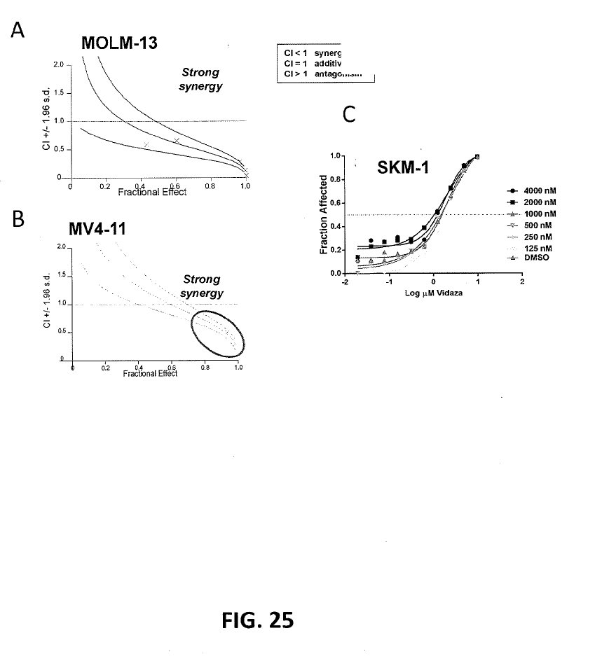

The results showed that Compound A2 acts synergistically with the AML standard of care agents cytarabine and Daunorubicinin Molm-13 and MV4-11 MLL-rearranged cell lines. Moreover, a persistent combination benefit was observed even when Compound A2 was washed out prior to the addition of the standard of care agents (FIG. 18), suggesting that Compound A2 sets up a durable altered chromatin state that enhances the effect of chemotherapeutic agents in MLL-rearranged cells. The combination of Compound A2 with other chromatin modifying drugs also revealed a consistent combination benefit including synergy with DNA hypomethylating agents.

In summary, the results indicate that Compound A2 is highly efficacious as a single agent and is synergistic with other anticancer agents including AML standard of care drugs and DNA hypomethylating agents in MLL-rearranged cells.

### EXAMPLE 3

### Example DOT1L Inhibitor Compound A2 Displays Synergistic Antiproliferative Activity in Combination with Standard of Care Drugs or DNA Hypomethylating Agents in MLL-Rearranged Leukemia Cells

Compound A2 is a small molecule inhibitor of the histone methyltransferase DOT1L that is currently under clinical investigation as a potential therapy for acute leukemias bearing MLL-rearrangements. Gene knockout and small molecule inhibitor studies have demonstrated that DOT1L is required for MLL-fusion protein—mediated leukemogenesis in model systems. In preclinical studies Compound A2 promoted cell killing of acute leukemia lines bearing MLL translocations in vitro while sparing those without MLL gene translocations and also caused sustained tumor regressions in a rat xenograft model of MLL-rearranged leukemia [Daigle et al. Blood 2013]. To support potential future clinical scenarios, the activity of Compound A2 in combination with current standard of care agents for acute leukemias as well as other chromatin modifying drugs was evaluated in cell proliferation assays with three human acute leukemia cell lines; Molm-13 (MLL-AF9 expressing acute myeloid leukemia (AML)), MV4-11 (MLL-AF4 expressing acute biphenotypic leukemia cell line) and SKM-1 (non-MLL-rearranged AML). Here is established a high density combination platform suitable for testing the anti-proliferative activity of a complete titration matrix of two agents with multiple replicate points to enable generation of statistically meaningful results. This platform was used to evaluate the anti-proliferative effects of Compound A2 combinations tested in a co-treatment model in which the second agent was added along with Compound A2 at the beginning of the assay, or in a pre-treatment model in which cells were incubated for several days in the presence of Compound A2 prior to the addition of the second agent. The drug combination analysis was performed using the Chou-Talalay method [Chou TC Pharmacological Reviews 2006]. Graphs representing values of combination index (CI) versus Fractional effect (Fa) known as Fa-CI plots were generated and synergy was evaluated. Drug synergy was statistically defined by CI values less than 1, antagonism by CI>1 and additive effect by CI equal to 1.

The results showed that Compound A2 acts synergistically with the AML standard of care agents cytarabine or daunorubicin in Molm-13 and MV4-11 MLL-rearranged cell lines. However, in the non-rearranged SKM-1 cell line Compound A2 had no effect alone and did not act synergistically with cytarabine or daunorubicin.

Moreover, a persistent combination benefit was observed even when Compound A2 was washed out prior to the addition of the standard of care agents suggesting that Compound A2 sets up a durable altered chromatin state that enhances the effect of chemotherapeutic agents in MLL-rearranged cells.

Evaluation of Compound A2 in conjunction with other chromatin modifying drugs also revealed a consistent combination benefit including synergy with DNA hypomethylating agents.

In summary, the results presented herein indicate that Compound A2 is highly efficacious as a single agent and is synergistic with other anticancer agents including AML standard of care drugs and DNA hypomethylating agents in MLL-rearranged cells.

### Methods:

### A) Pre-Treatment Model in 96-Well Format:

Human leukemia cell lines were pretreated in flasks with 7 concentrations of Compound A2 or DMSO for 4 (MV4-11 cells) or 7 days (MOLM-13 cells). Cells were then counted and reseeded with, or without Compound A2 (Compound A2 washout) in 96-well plates at a constant cell density in the presence of increasing concentrations of a second agent for an additional 3 days. The HP-D300 digital dispenser (Tecan) was used to dispense compounds in a combinatorial matrix. Cells were treated with concentrations of Compound A2 and standard of care agent which were bracketed above and below the IC₅₀ of each compound alone. Cell viability was measured via ATP content using CellTiter-Glo® (Promega).

### B) Co-Treatment Model in 96-Well Format:

Human leukemia cell lines were treated with matrix of 7 concentrations of Compound A2 and 9 concentrations of compound of interest for 7 days. Viability was determined using CellTiter-Glo® (Promega).

### C) Pre-Treatment Model for Mechanism of Cell Death Studies:

MOLM-13 cells were pretreated in flasks with 7 concentrations of Compound A2 or DMSO vehicle control for 7 days. Cells were then counted and reseeded in 96-well plates at a constant cell density in the presence of Compound A2 and Ara-C at concentrations previously demonstrated to give synergistic cell killing activity and incubated for an additional 3 or 7 days. A Guava EasyCyte HT™ flow cytometer was used to measure DNA content, Annexin V staining and cell surface expression of CD14 and CD11b markers on Days 10 and 14.

TABLE 2
Summary of Combination Studies with Compound A2 in AML Cell Lines

| | | | | SKM-1 |
|---|---|---|---|---|
| | | | MOLM- | AML: |
| | | MV4-11 | 13 | Non- |
| | | MLL-AF4 | MLL-AF9 | rearranged |
| AML Standard of | Ara-C | Strong | Synergy | No |
| Care Agents | | Synergy | | Combination |
| | | | | Benefit |
| | Daunorubicin | Synergy | Synergy | No |
| | | | | Combination |
| | | | | Benefit |
| DNA | Azacitidine | Synergy | Synergy | No |
| Methyltransferase | | | | Combination |
| Inhibitors | | | | Benefit |
| | Decitabine | Synergy | Synergy | No |
| | | | | Combination |
| | | | | Benefit |
| Bromodomain | IBET-151 | Synergy | Synergy | IC50 not |
| Inhibitors | | | | achieved |
| | JQ1 | Additive | Additive | TBD |

TABLE 3
Summary of Combination Studies with Compound A2 in AML Cell Lines

| | | EOL-1 | KOPM-88 |
|---|---|---|---|
| | | (MLL- | (MLL- |
| | | PTD) | PTD) |
| AML Standard | Ara-C/Cytarabine | Synergy | Synergy |
| of Care Agents | Daunorubicin | Synergy | Synergy |
| Hypomethlyating | Azacitidine | Synergy | Synergy |
| Agent | | | |

Combination benefit with Compound A2 is achieved with all drugs tested in MLL-rearranged leukemia cell lines Molm-13 and MV4-11 and MLL-PTD cell lines EOL-1 and KOPM-88 sparing the non-rearranged SKM-1 cell line.

In summary, the present study demonstrates that:
(1) Compound A2 acts synergistically with the AML SOC drugs Ara-C and daunorubicin to induce a strong antiproliferative response that is selective for MLL-rearranged leukemia cells;
(2) Synergy is observed even when Compound A2 is washed out prior to the addition of Ara-C and daunorubicin;
(3) Initial studies suggest that the concurrent induction of apoptosis and differentiation underlies the combination benefit observed with SOC drugs in the MLL-rearranged leukemia cell line MOLM-13; and
(4) Synergistic anti-proliferative activity in MLL-rearranged leukemia cell lines is also observed when Compound A2 is used in combination with several chromatin modifying agents, including the DNA-methyltransferase inhibitors azacytidine and decitabine and the bromodomain inhibitor i-BET.

Taken together these studies suggest that Compound A2 sets up an altered chromatin and/or gene expression state in MLL-rearranged cells that dramatically potentiates the cytotoxic effects of current AML SOC drugs.

### EXAMPLE 4

### Synergistic Activity of Ara-C and Compound A2

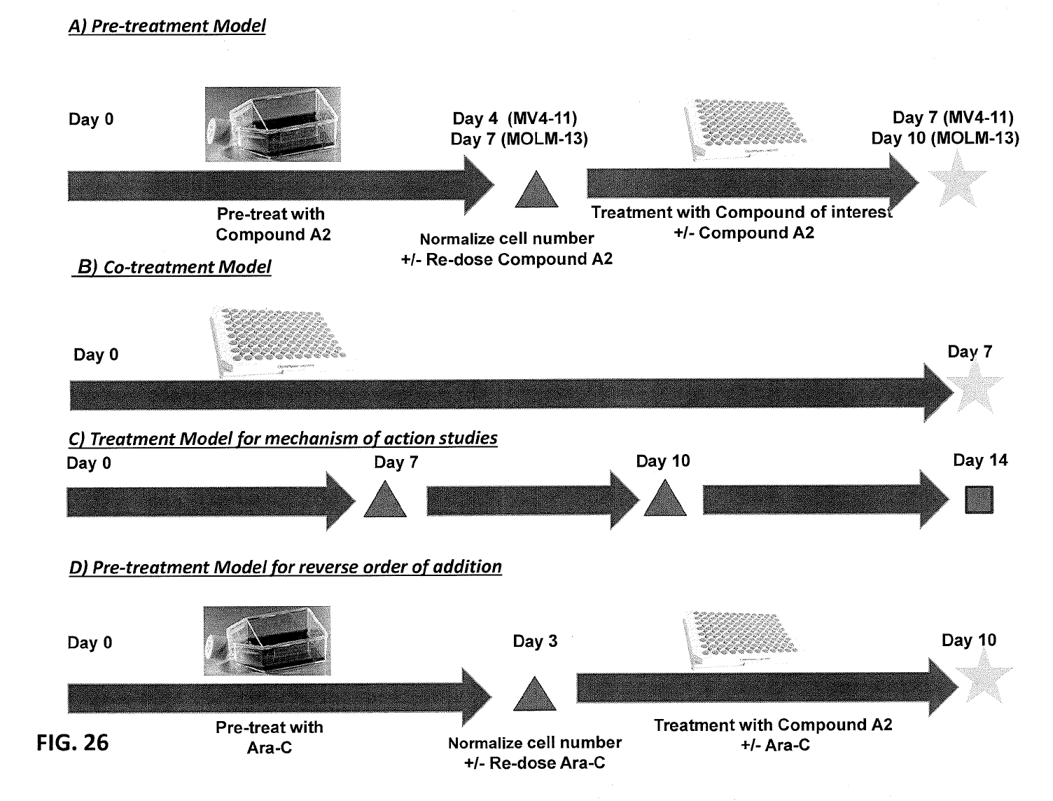

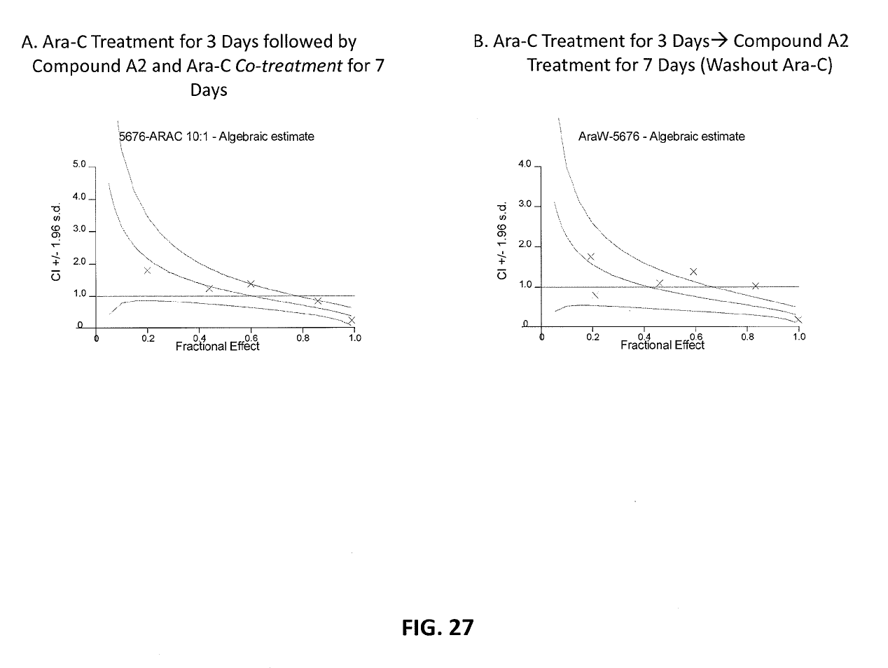

As shown in FIG. 26D, pre-treatment model with reverse order of addition in 96-well format is carried out as follows.

MOLM-13 cells were pretreated with 9 concentrations of Ara-C or DMSO for 3 days. Cells were then counted and reseeded with or without Ara-C (Ara-C washout) in 96-well plates at a constant cell density in the presence of increasing concentrations of Compound A2 for an additional 7 days.

The HP-D300 digital dispenser (Tecan) was used to dispense Compound A2 and Ara-C in a combinatorial matrix. Cells were treated with concentrations of Compound A2 and Ara-C bracketed above and below the IC₅₀ of each compound alone. Cell viability was measured via ATP content using CellTiter-Glo® (Promega).

### Results:

Synergy is observed when cells are pretreated with Ara-C followed by cotreatment with Compound A2. Combination benefit is maintained when Ara-C is washed out prior to treatment with Compound A2.

### EXAMPLE 5

### Compound A2 Induces a Synergistic and Durable Antiproliferative Effect in Combination with AML Standard of Care Drugs

Materials and methods

### Cell Lines

The acute myelogenous leukemia cell line MV4-11 (MLL-AF4) (CRL-9591) was obtained from American Type Culture Collection (ATCC), Manassas, Va. and both MOLM-13 (MLL-AF9) (ACC 554) and SKM-1 (ACC 547) cells were obtained from Leibniz Institute DSMZ-German Collection of Microorganisms and Cell Cultures, Braunschweig, Germany. MV4-11 cells were maintained in IMDM supplemented with 10% fetal bovine serum. MOLM-13 and SKM-1 cells were maintained in Roswell Park Memorial Institute medium (RPMI) supplemented with 10% fetal bovine serum. They were cultured in flasks or plates in a humidified 5% CO₂ atmosphere.

### Proliferation Assays and Calculation of Synergism

Proliferation studies were performed using MOLM-13, MV4-11 and SKM-1 cell lines in vitro to evaluate the cancer cell killing effect of a combination of two agents together on cell growth. Initial proliferation studies were performed to determine the IC₅₀ values of a given compound in each cell line. The cell counts were measured by ATP quantitation using the Promega Cell Titer Glo kit and luminescence values correspond to the amount of ATP in a given well.

These studies were performed to evaluate both the combinatorial effect of compounds on cell killing and the durability of the effect by washing out one of the agents. Compounds were tested in combination with Compound A2 to study their effect on cell proliferation in either a 4+3 model where cells were pretreated with increasing concentrations of Compound A2 for 4 days, followed by a co-treatment with Compound A2 with test article for 3 days or a 7 day co-treatment model.

In addition, the effect of sequence of addition of compounds was studied by measuring the ten day proliferation of cells pretreated with Ara-C in a 3+7 model. This experiment was performed by first pretreating MOLM-13 cells with increasing concentrations of Ara-C for 3 days. Ara-C was then washed out, the cell numbers were normalized and either Compound A2 alone or Compound A2 cotreatment in a matrix format with Ara-C was performed. The cells were then normalized on day 3, followed by washout of Ara-C or cotreatment of cells with Compound A2 and Ara-C for 7 days.

Compounds evaluated for synergy in the co-treatment phase were tested in a range which was bracketed around the IC₅₀'s. The compounds were plated to a 96 well plate in a matrix format which includes increasing concentrations of each drug in the combination in a constant ratio, in addition to the effect of each compound alone in the study. Cells were seeded and grown in the log-linear phase for 3 or 7 days in the cotreatment phase. Maximum and minimum inhibition (DMSO alone) controls were used in each plate to calculate fraction affected (Fa) of a test well. DMSO concentration was kept at 0.1% v/v. The drug combination analysis was performed utilizing the Chou-Talalay method. Synergy was determined using the software package Calcusyn by Biosoft. The combination index (CI) is the term used to describe the level of synergy or antagonism in a given test system. A combination index less than one indicates synergy, and a CI greater than one indicates antagonism.

### Cell Treatment for Analysis of Mechanism of Cell Death Studies

On Day 0 MOLM-13 cells are seeded at 3,000 cells/mL. On Day 7 and Day 10 MOLM-13 cells are counted and reseeded at 50,000 cells/mL. MOLM-13 cells were treated with various concentrations of compounds as a single agent or in combination with AraC or Daunorubicin. Day 1-7 cells were only treated with Compound A2. On Day 7 cells reseeded and redosed with Compound A2 alone or in combination with AraC or Daunorubicin as described below. On Day 10 They were redosed again. On Day 14 the experiment was terminated. Cells were sampled for CD14 and CD11b analysis on Days 7, 10 and 14.

### Flow Cytometric Analysis of Cell Cycle and Annexin V

To evaluate the fraction of cells in each cell cycle, flow cytometric analysis was performed. FACS analysis for detection of cell death by apoptosis, and cell cycle was performed. Cells were treated alone with Compound A2 or in combination. To allow for simultaneous analysis of cell cycle and apoptosis, cells were treated alone or in combination with Compound A2.

Cells were harvested on days 7, 10 and 14 and split to allow simultaneous analysis of cell cycle and Annexin V staining. Apoptosis was determined using the Guava Nexin Assay (Millipore 4500-0450) and cells were prepared according to the manufacturer's recommendations. Samples were analyzed using the Guava EasyCyte Plus System (Millipore). Cells for cell cycle analysis were pelleted by centrifugation at 200×g for 5 minutes at 4° C., washed twice with ice cold PBS then fixed with 70% ice cold ethanol. All samples were analyzed together at end of experiment. Following fixation cells were washed with PBS and stained with the Guava cell cycle reagent (Millipore 4500-0220) for 30 minutes. Samples were analyzed using the Guava EasyCyte Plus System (Millipore).

### Analysis of CD11b and CD14 Expression by Flow Cytometry

To analyze the degree of differentiation, MOLM-13 cells were incubated in the presence of 0.1% DMSO or previously stated concentrations of Compound A2, Ara-C, Daunorubicin or in combination. On day 7, 10, and 14, cells were collected for analysis. The cells were prepared by washing twice in PBS, followed by fixation in 4% formaldehyde for ten minutes at 37° C. After fixation cells were washed and blocked with blocking buffer for 10 minutes at room temperature. Cells were then incubated in presence of anti-CD14, anti-CD11b or anti-IgG antibody for 1 hour at room temperature while rotating. Cells were washed, re-suspended in PBS and 5,000 events were analyzed using ExpressPro software on the GuavaCyte Plus System.

### Analysis of CD11b and Caspase Cleavage by High Content Screening

To further analyze the cell population for differentiation or markers of apoptotic cell death, MOLM-13 cells were collected on days 5, 7, 8, 9, 10, 11, 12 and 14 for imaging. Cells were incubated with test articles, and at each time point, cells were collected, washed once in PBS and re-suspended in 0.5% BSA+PBS blocking buffer. CD11b antibody, at a dilution of 1:12.5, was incubated with the cells for 15 minutes at 37° C. in the dark at room temperature while rotating. Medium A was added and the cells were incubated for an additional 15 minutes. After one wash with PBS+0.1% NaN₃+5% FBS cells were re-suspended in Medium B from the Fix and Perm kit. DAPI at a 1:100,000 dilution and second antibody (Caspase-3 or H2A.X) at a 1:50 dilution were added and cells incubated for 20 minutes at room temperature in the dark. After the last incubation, cells were washed one time in PBS+0.1% NaN₃+5% FBS and re-suspended in 150 μL of PBS, allowed to settle on the plate for about 30-60 minutes then imaged.

The drug combination analysis was performed using the Chou-Talalay method. Graphs representing values of combination index (CI) versus Fractional effect (Fa) known as Fa-CI plots were generated and synergy was evaluated. Drug synergy was statistically defined by CI values less than 1, antagonism by CI>1 and additive effect by CI equal to 1.

### Results

Compound A2 Induces a Synergistic and Durable Antiproliferative Effect in Combination with AML Standard of Care Drugs

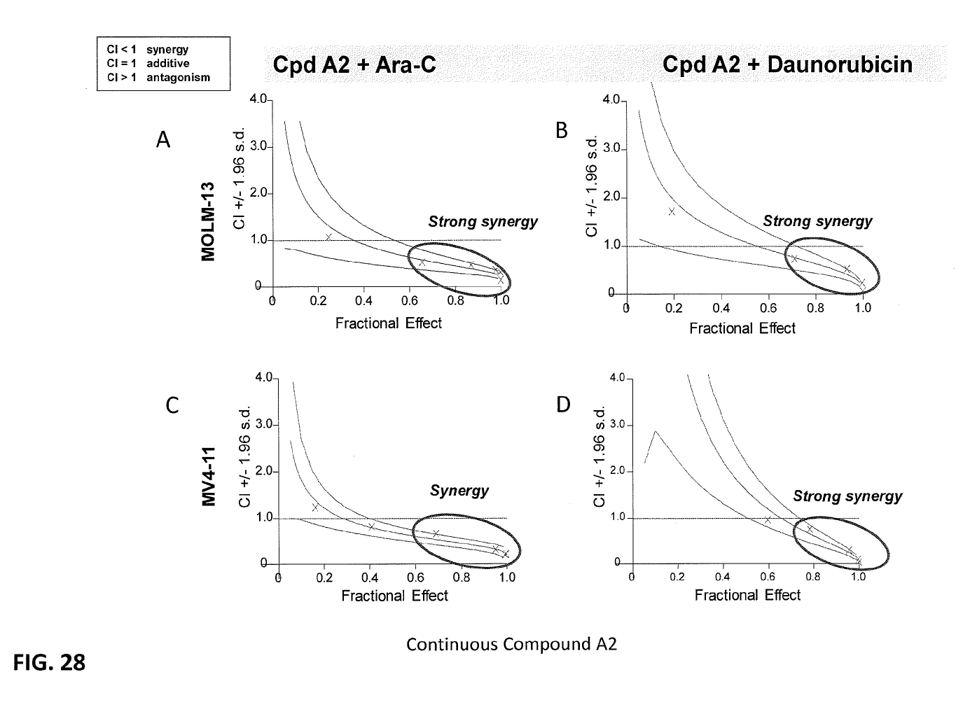

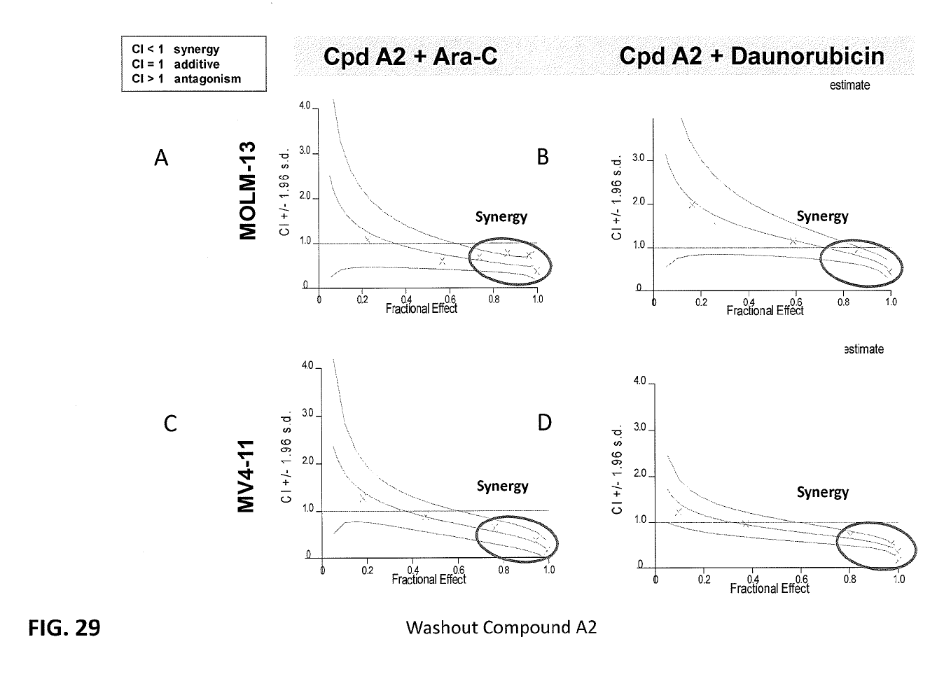

Compound A2 demonstrates synergistic antiproliferative activity in combination with two standard of care (SOC) drugs for AML, cytarabine and daunorubicin in the MLL-rearranged leukemia cell lines MOLM-13 and MV4-11 (FIG. 28). Cells were treated according to the pre-treatment model described in above (i.e., no Compound A2 washout). The synergistic anti-proliferative activity of Compound A2 in combination with AML SOC agents was also observed when cells were treated according to the co-treatment model. Intriguingly, this synergistic anti-proliferative activity was maintained in MOLM-13 and MV4-11MLL-rearranged cells even when Compound A2 is removed (i.e., washed out) prior to the addition of the SOC agent (FIG. 29). These data are remarkable in that they imply a durable reprogramming of the epigenetic status of these cells by Compound A2 that renders them more acutely sensitive to chemotherapeutic agents, even when the DOT1L inhibitor has been removed from the cellular environment. This result is consistent with the kinetics of Compound A2 effect on histone methylation at the DOT1L substrate site, H3K79 (Daigle et al, 2013). In previous studies, it was shown that four days of treatment with Compound A2 is sufficient to deplete cellular levels of H3K79me2 by ≥80%. When Compound A2 was then removed, by wash out from these cells, no recovery of H3K79 methylation was observed for 3 days after wash out. After this 3-day latency period, the level of H3K79me2 slowly returned to pretreatment levels over the course of an additional 4 days. Hence, treatment of MLL-rearranged cells with Compound A2 results in durable inhibition of H3K79 methylation which in turn results in sensitization of these cells to chemotherapy-induced cell killing. These results offer the possibility of a highly flexible dosing schedule for combinations of Compound A2 and chemotherapies.

The synergistic effects of Compound A2 and chemotherapeutic agents were very similar in both MLL-rearranged cells tested (MV4-11 and MOLM-13). In the interest of clarity and brevity, below is presented representative data for MOLM-13 cells only. In all cases, similar results were observed in the MV4-11 cell line as well.

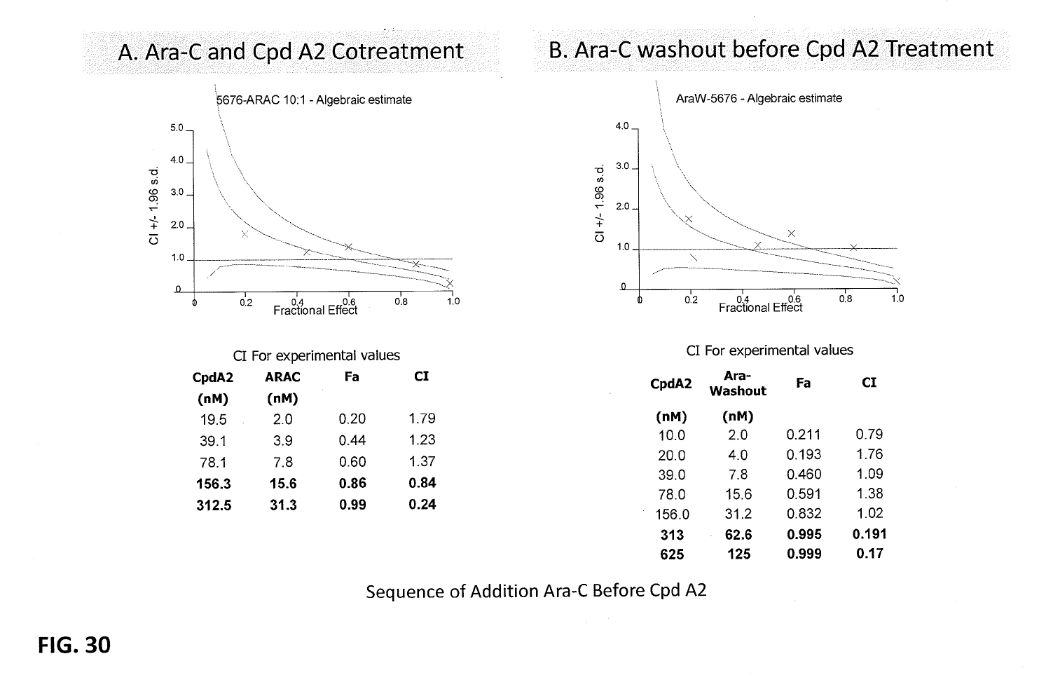

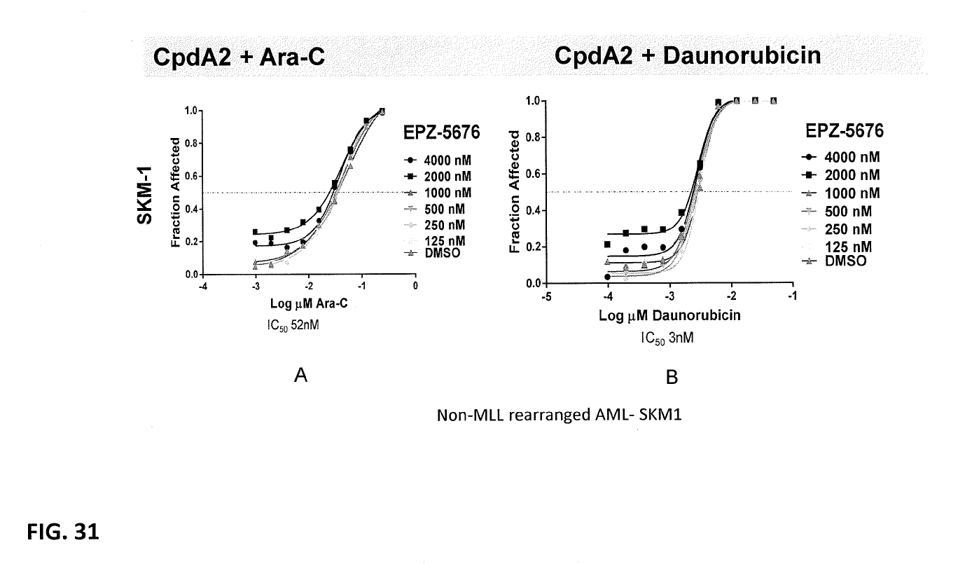

To test further the flexibility of dosing schedules that might afford synergistic cell killing, MOLM-13 cells were pretreated with the chemotherapeutic agent cytarabine for 3 days, washed this drug out and then treated the cells with Compound A2 for an additional 7 days. As illustrated in FIG. 30, this sequential treatment schedule resulted in essentially the same level of synergistic cell killing as seen when both drugs were co-administered to cells simultaneously.

While both single agent activity and strong synergy with cytarabine and daunorubicin were seen for Compound A2 in the MLL-rearranged cell lines MV4-11 and MOLM-13, no effect of Compound A2 was observed in the non-MLL-rearranged leukemia cell line SKM-1. Compound A2 showed no single agent activity in this latter cell line and did not affect the antiproliferative activity of either chemotherapeutic agent in this cell line either (data not shown). The lack of activity of Compound A2 in SKM-1 cells is completely consistent with the proposed mechanism of action of this drug. In previous studies it was demonstrated that while Compound A2 inhibits intracellular DOT1L activity—as evidenced by concentration-dependent inhibition of H3K79 methylation—across a spectrum of AML cell lines, this enzyme inhibition only translates into an antiproliferative effect for those leukemia cells bearing an 11q23 chromosomal translocation.

Compound A2 Increases Expression of Differentiation Markers and Apoptosis as Single Agent and in Combination with AML Standard of Care Drugs

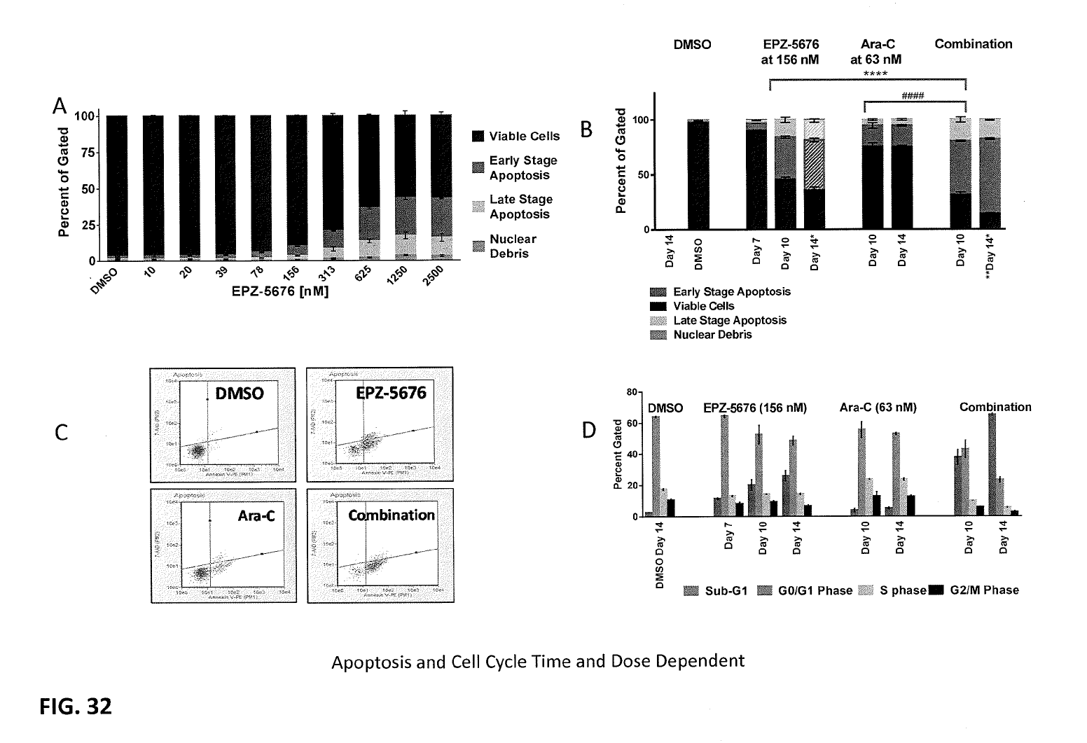

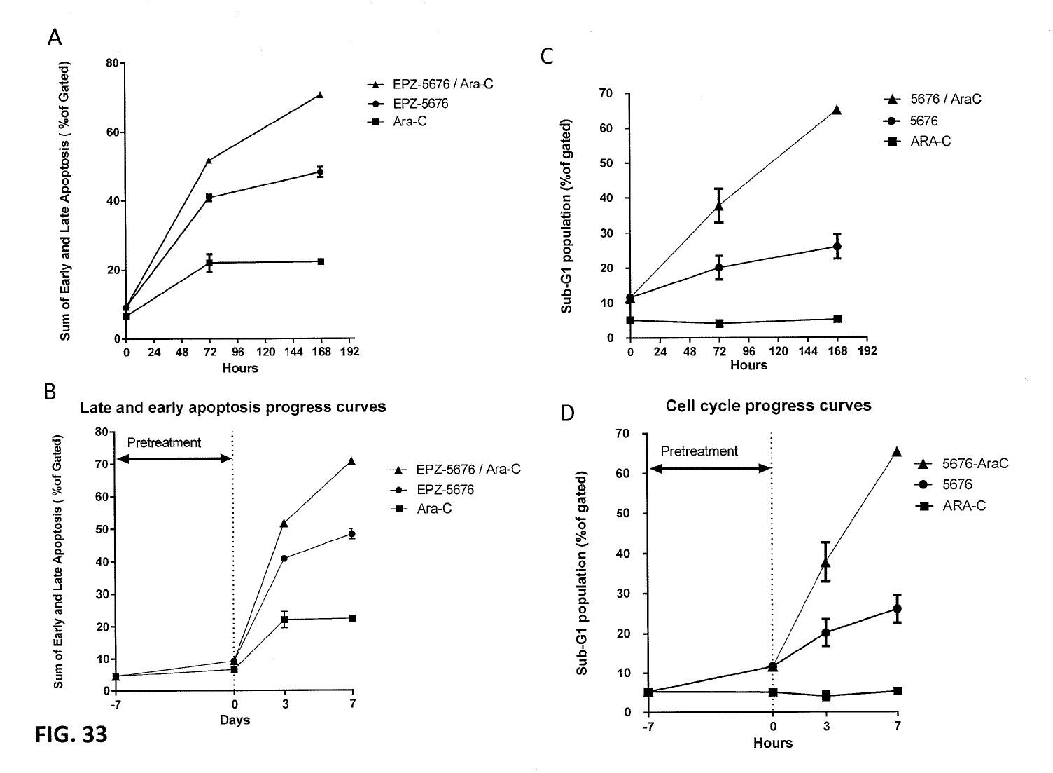

Compound A2 induces a concentration-dependent increase in apoptotic cells (as measured by Annexin-V staining) after 7 days of treatment of MOLM-13 cells as a single agent. As illustrated in FIG. 32A, the total content of viable cells decreases with Compound A2 concentration according to a classic Langmuir isotherm, with a midpoint value (EC₅₀) of 364±18 nM and this trend is exactly mirrored by the increasing content of apoptotic cells (sum of early and late stage apoptosis). The kinetics apoptosis induction was measured at fixed time points over a 14 day course of treatment for MOLM-13 cells treated with DMSO (as a control), 156 nM Compound A2, 63 nM cytarabine (Ara-C) or a combination of Compound A2 and Ara-C (at the same concentrations as for the single agent treatments). Ara-C by itself induced a modest increase in apoptotic cell population over the 14 day treatment period, while Compound A2 lead to much more robust induction of apoptosis over the same time course. The combination of the two drugs led to enhance apoptosis in the MOLM-13 cells (FIG. 32B). Apoptotic cell content was also assessed by measuring the percent of cells in the sub-G1 phase of the cell cycle. FIG. 32C illustrates the distribution of cell cycle stages at various time points for MOLM-13 cells treated with DMSO (control), 156 nM Compound A2, 63 nM Ara-C or a combination of Compound A2 and Ara-C. The data for the sub-G1 cell population is also graphed as a kinetic plot in FIG. 32D. This plot makes clear that Ara-C treatment alone has minimal effect of the sub-G1 population of MOLM-13 cells over the 14 day treatment course, while treatment with Compound A2 leads to a moderate, time-dependent increase in sub-G1 population. When Compound A2 and Ara-C are combined, a significant increase in the population of sub-G1 cells at 10 and 14 days is realized with a concomitant increase in the rate of sub-G1 population growth as well. Similar results were observed when Compound A2 was combined with daunorubicin.

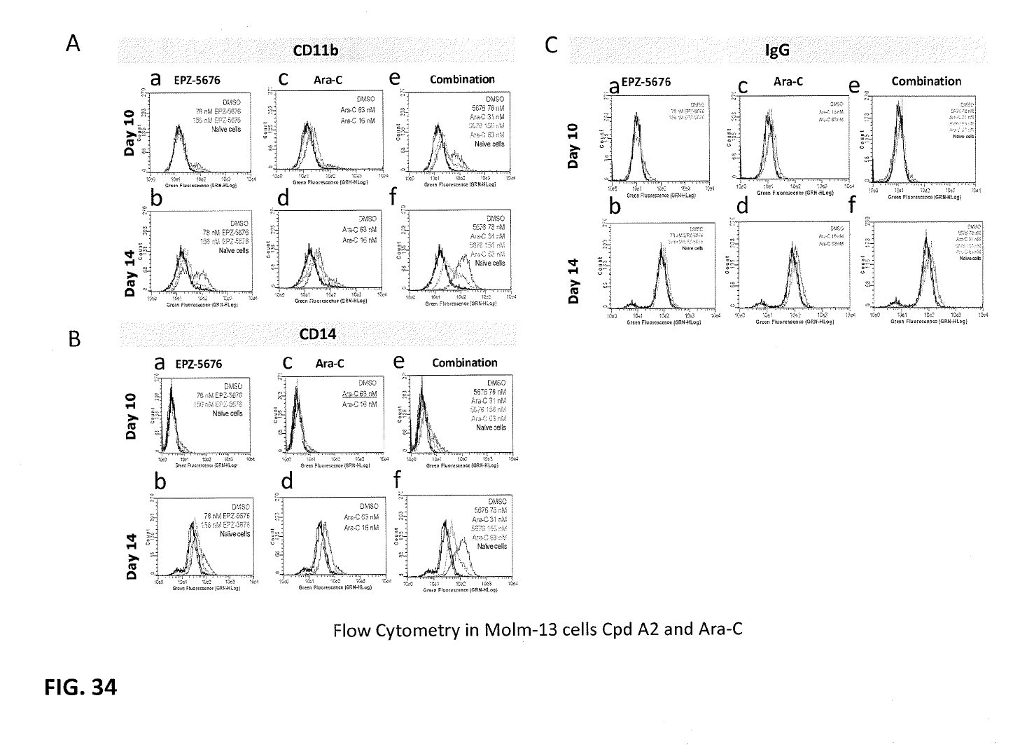

In addition to driving apoptotic cell death, Compound A2, Ara-C as single agents and in combination promote time and concentration dependent up-regulation of the differentiation markers CD11b and CD14 (Figure. 34) in MLL-rearranged MOLM-13 cells. The same effect was observed with daunorubicin as a single agent and in combination with Compound A2.

The degree of differentiation marker upregulation was greater with the combination of agents than with either agent alone. This significant upregulation was also shown by gene expression analysis of differentiation markers in MOLM-13 cells treated alone or in combination with Compound A2 and either Ara-C or daunorubicin. Without being bound by theory, these results demonstrate that the synergistic antiproliferative activity observed by combining Compound A2 with AML SOC agents is due to an enhanced ability of drug combinations over single agents to induce apoptosis and differentiation in MLL-r cells.

Compound A2 Demonstrates Combination Benefit with Acute Lymphoblastic Leukemia Standard of Care Drugs

MLL-r is also found in acute lymphoblastic leukemia (ALL) and is primarily associated with infants (children younger than 12 months). This subset of ALL has a poor prognosis when compared with the ALL patients without the 11q23 translocation. Long-term event-free survival in infants harboring MLL-r has been reported to be between 28 and 45%. These rates are much lower than non-MLL-r patients who have survival rates approaching 90% (Pieters et al., Lancet 370:240-250, 2007; Bhojwani et al., Clin Lymphoma Myeloma 9 (Suppl 3):S222-S230 10.3816/CLM.2009.s.016, 2009; Inaba et al., Lancet 381:1943-1955, 2013). Similar to the AML SOC, experiments were performed to evaluate the combination of Compound A2 with current ALL therapies that include mitoxantrone, methotrexate, mafosfamide, prednisolone, and vincristine (Pieters et al., 2007; Inaba et al., 2013). The results of these combinations are summarized in Table 4. Synergism or additive effects were observed with all of the ALL SOC agents in combination with Compound A2 with the exception of prednisolone, where antagonism was observed in MLL-r cell lines. No enhancement of the antiproliferative single-agent activity of ALL SOC drugs was seen when combined with Compound A2 in the non-MLL-r cell line SKM-1 with the exception of prednisolone, where enhanced antiproliferative activity was observed in the presence of Compound A2 concentrations greater than 1000 nM. The basis for this enhancement in prednisolone activity is unknown; however, it is noteworthy that these Compound A2 concentrations used are much higher than those required for maximal efficacy in preclinical MLL-r models.

Compound A2 Demonstrates Strong Synergy with DNMT Inhibitors in MLL-Rearranged Cell Lines

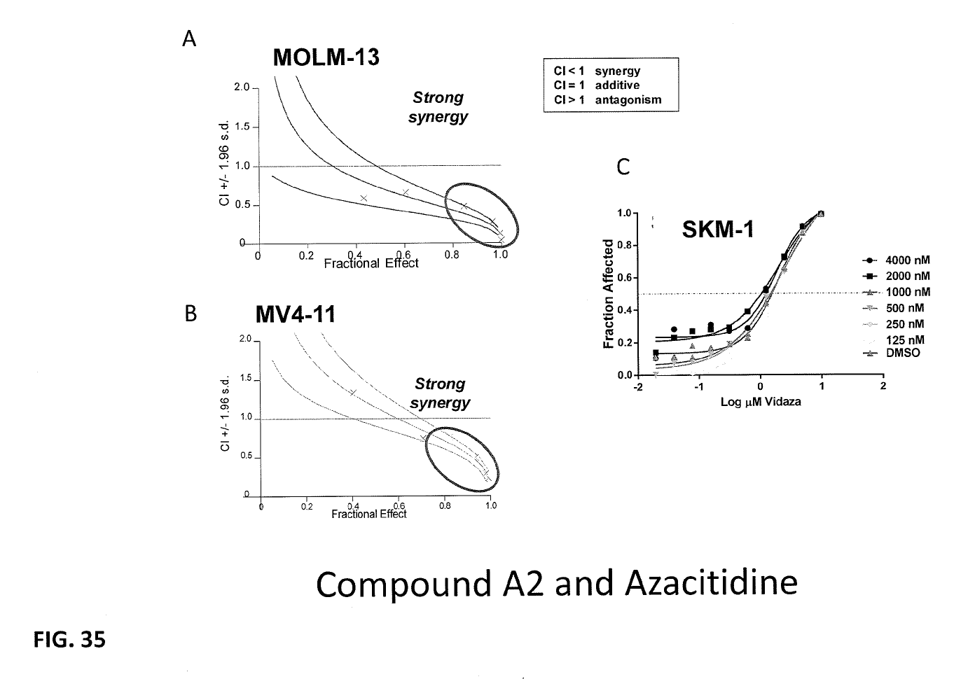

Compound A2 represents the first protein methyltransferase (PMT) inhibitor to be tested in human clinical trials. The PMT target class effects chromatin remodeling and gene transcriptional programming by site-specific methylation of lysine residues on histones H3 and H4; in the case of DOT1L, the enzyme uniquely catalyzes the methylation of a single histone site, H3K79. There is considerable evidence that epigenetic regulation of gene transcriptional results from the combinatorial effects of distinct covalent modifications of chromatin components, including histone methylation, histone acetylation, other covalent histone modifications and direct methylation of chromosomal DNA at CpG islands by the DNA methyltransfersases (DNMTs). Next, the impact of combining the PMT inhibitor Compound A2 in combination with other compounds that affect their pharmacology was tested by inhibition of other chromatin modifying enzymes, such as histone deacetylases (HDAC) histone demethylases (HDMs), acetyl-lysine reader domains (bromodomains) and DNA methyltransferases (DNMTs). The results of these combinations are summarized in Table 4 and demonstrate a range of effects from antagonism with some HDAC inhibitors in the context of MV4-11 cells to synergy. Among these other chromatin modifying enzyme inhibitors, the DNMT inhibitors decitabine and azacytidine demonstrated synergistic anti-proliferative activity in MLL-rearranged cells when combined with Compound A2. In contrast, and again consistent with the mechanism of action of Compound A2, this compound had no impact on the antiproliferative activity of either DNMT inhibitor when tested in the non-MLL-rearranged leukemia cell line SKM-1 (Table 4). FIG. 35 illustrates representative data for the strong synergistic effects of combining Azacitidine and Compound A2 in MV4-11 and MOLM-13 cell lines. Similar synergy was also seen in these cell lines when Compound A2 was combined with another DNMT inhibitor, decitabine (Table 4).

TABLE 4
Summary of Combinations Evaluated in 7 Day Cotreatment Model

| Rationale/ | | MOLM-13 | MV4-11 |
|---|---|---|---|
| Class | Compound | (MLL-AF9) | (MLL-AF4) |
| AML SOC | Ara-C | Strong Synergy | Strong |
| | | | Synergy |
| | Daunorubicin | Synergy | Strong |
| | | | Synergy |
| | Mercaptopurine | Additive--> Synergy | Additive |
| DNMTi | Azacitidine | Strong Synergy | Synergy |
| | Decitabine | Synergy | Synergy |
| HDACi | Vorinostat | Additive/Synergy | Antagonistic |
| | Panobinostat | Synergy | Antagonistic |
| HDMi | Tranylcypromine | Strong Synergy | Synergy |
| | LSD1 inhibitor II | Nearly Additive | Synergy |
| BRDi | IBET-151 | Synergy | Strong |
| | | | Synergy |
| | JQ1 | Additive | Additive |
| ALL SOC | Mitoxantrone | Synergy | Synergy |
| | Methotrexate | Additive | Additive |
| | Mafosfamide | Strong Synergy | Strong |
| | | | Synergy |
| | Prednisolone | Antagonistic | Antagonistic |
| | Vincristine | Additive | Additive |
| AML/ALL | Fludarabine | Synergy | Additive--> |
| SOC | | | Synergy |
| Immunomod. | Lenalidomide | Combination Benefit | Combination |
| Agent | | | Benefit |
| Bcl-2i | Navitoclax | Synergy | Synergy |
| | Obatoclax | Additive | |
| | ABT-199 | Additive--> Synergy | Synergy |
| MEK1/2i | Trametinib | Synergy | Additiveà |
| | | | Synergy |
| FLTi | Quizartinib | Synergy | |
| | Midostaurin | Additive--> Synergy | Synergy |
| Proteasome i | Velcade | Combination Benefit | Combination |
| | | | Benefit |
| Antimetabolite | Hydroxyurea | Synergy | Synergy |
| | Clofarabine | Synergy | Synergy |
| MLL-Binding | Menin-MLL | Synergy | Synergy |
| Partner | inhibitor MI-2 | | |
| Inhibitor | | | |
| SIRT1 | SRT-1720 | n/a | Synergy |
| activator | | | |
| BTKi | Ibrutinib | Antagonistic/Additive | Synergy |
| CDK4/6 | Palbociclib | Synergy | Synergy |
| Nitrofuran | Furazolidone | Combination Benefit | Synergy |

Compound A2 in combination with other compounds that affect their pharmacology was tested. The results of these combinations are summarized in Table 5 below.

TABLE 5
Summary of Combinations Evaluated in 7 Day Cotreatment Model

| Modality | Compound | MOLM13 | MV4-11 |
|---|---|---|---|
| ATRi | AZ20 | Synergy | |
| AKTi Pan | MK2206 | Additive | |
| allosteric | | | |
| Dual | BEZ235 | Additive | |
| PI3k/MTORi | | | |
| PPAR antagonist | T0070907 | Additive | |
| EZH2i | Compound E10 | Synergy | Combination |
| | | | Benefit |
| | Romidepsin | | No change IC50 |
| Farnesyl | Tipifarnib | | Synergy |
| Transferase | | | |
| inhibitor | | | |
| PLK1i | Volasertib | — | Antagonstic |

Further aspects, embodiments, and elements of the disclosure are described in Klaus et al “DOT1L Inhibitor EPZ-5676 Displays Synergistic Antiproliferative Activity in Combination with Standard of Care Drugs and Hypomethylating Agents in MLL-Rearranged Leukemia Cells” J Pharmacol Exp Ther 350:1-11, September 2014, the contents of which are hereby incorporated by reference in its entirety.

### EXAMPLE 6

### Additional Combination Studies

### Methods

MOLM-13 cells or SKM-1 cells were pre-treated with 300 nM of EPZ-5676 (i.e., Compound A2) or DMSO in T175 flasks for a 4-day pre-treatment time. Cells were split using EPZ-5676 or DMSO containing growth media and further incubated for an additional 3-day pre-treatment time. Cells were finally seeded in growth media containing EPZ-5676 or DMSO in 384-well plates at 500 cell/well density. Cells were then equilibrated in incubators for 24 hours before treatment with a second compound. Treated assay plates were incubated with a second compound for 72 hours. After this time, plates were developed for endpoint analysis using ATPLite to measure ATP content, which is used as an indicator of cell viability.

A combination of EPZ-5676 and a second compound was considered synergistic if the IC₅₀ value of the second compound decreased by 2-fold or more when EPZ-5676 was added as compared to the DMSO control.

The results of these combinations are summarized in Table 6 below. “N/D” means that IC₅₀ of the second compound could not be determined for both conditions.

TABLE 6
Summary of Combination Studies

| 2nd Compound | Mechanism | Target | MOLM-13 | SKM-1 |
|---|---|---|---|---|
| 10-Hydroxycamptothecin | DNA_function | TOP2A inhib | Synergy | No |
| | | | | combination |
| | | | | benefit |
| 17-DMAG | Protein_proc | HSP90 inhib | No | No |
| | | | combination | combination |
| | | | benefit | benefit |
| 1-Azakenpaullone | Signal_apop | GSK3b inhib | No | N/D |
| | | | combination | |
| | | | benefit | |
| 6-Aminonicotinamide | Metabolism | NAD related | No | N/D |
| | | | combination | |
| | | | benefit | |
| 6-mercaptopurine | Metabolism | Purine | No | No |
| monohydrate | | | combination | combination |
| | | | benefit | benefit |
| A 769662 | Signal_kinase | AMP kinase active | N/D | N/D |
| ABT-737 | Signal_apop | BCL-2 antag | No | Synergy |
| | | | combination | |
| | | | benefit | |
| ABT-888 | DNA_repair | PARP inhib | N/D | N/D |
| AC-220 | Signal_kinase | IKK1/IKK2 inhib | No | No |
| | | | combination | combination |
| | | | benefit | benefit |
| Alitretinoin | Regulation | RAR/RXR bind | Synergy | No |
| | | | | combination |
| | | | | benefit |
| AMI-5 | Chromatin | HMT inhib | N/D | N/D |
| AP24534 | Signal_kinase | BCR-ABL1/Src inhib | No | No |
| | | | combination | combination |
| | | | benefit | benefit |
| AP26113 | Signal_kinase | ALK; FER; ROS/ROS1; FLT3; FES/FPS | No | No |
| | | | combination | combination |
| | | | benefit | benefit |
| Arsenic Trioxide | Signal_apop | PML/RARa inhib | No | 2.0 |
| | | | combination | |
| | | | benefit | |
| AST-1306 | Signal_grow | ErbB1; ErbB4 | N/D | N/D |
| AT7519 | Signal_cell cycle | CDK inhib | No | No |
| | | | combination | combination |
| | | | benefit | benefit |
| AT7867 | Signal_kinase | AKT; p70 S6K inhib | Synergy | No |
| | | | | combination |
| | | | | benefit |
| Auranofin | Metals | thioredoxin reductase | Synergy | No |
| | | | | combination |
| | | | | benefit |
| AVL-292 | Signal_kinase | BTK inhib | No | N/D |
| | | | combination | |
| | | | benefit | |
| AZD 5582 | Signal_apop | SMAC | No | N/D |
| dihydrochloride | | | combination | |
| | | | benefit | |
| AZD 8055 | Signal_kinase | MTOR inhib | No | Synergy |
| | | | combination | |
| | | | benefit | |
| AZD1152- | Signal_cell cycle | Aurora inhib | No | N/D |
| HQPA(Barasertib) | | | combination | |
| | | | benefit | |
| AZD6244 | Signal_kinase | MEK inhib | Synergy | No |
| | | | | combination |
| | | | | benefit |
| AZD7762 | Signal_cell cycle | Chk1; CHK2 inhib | No | No |
| | | | combination | combination |
| | | | benefit | benefit |
| Baricitinib | Signal_kinase | JAK inhib | Synergy | N/D |
| Bay 11-7082 | Signal_inflam | IKKA inhib | No | No |
| | | | combination | combination |
| | | | benefit | benefit |
| Bay 41-2272 | Cytoskeleton | sarcoglycan | No | N/D |
| | | | combination | |
| | | | benefit | |
| BAY 61-3606 | Signal_kinase | SYK inhib | No | No |
| Hydrochloride | | | combination | combination |
| | | | benefit | benefit |
| Belinostat | DNA_function | HDAC inhib | No | No |
| | | | combination | combination |
| | | | benefit | benefit |
| Bendamustine | DNA_damage | DNA alkylator | N/D | N/D |
| Hydrochloride | | | | |
| BEP800 | Protein_proc | HSP90 | Synergy | No |
| | | | | combination |
| | | | | benefit |
| Bexarotene | Regulation | RXRB bind | Synergy | Synergy |
| BGJ398 | Signal_grow | FGFR1; FGFR2; FGFR3; FGFR4 | No | No |
| | | | combination | combination |
| | | | benefit | benefit |
| BI 2536 | Signal_kinase | Plk1 inhib | No | No |
| | | | combination | combination |
| | | | benefit | benefit |
| BIIB021 | Protein_proc | HSP90 inhib | No | No |
| | | | combination | combination |
| | | | benefit | benefit |
| BIX 02189 | Signal_kinase | MEK5; ERK5 | No | N/D |
| | | | combination | |
| | | | benefit | |
| BIX01294 | Chromatin | G9a histone lysine methyltransferases | Synergy | No |
| | | | | combination |
| | | | | benefit |
| Bleomycin Sulfate | DNA_damage | DNA ligase inhib | Synergy | N/D |
| BML-275 | Signal_kinase | AMP kinase inhib | No | No |
| | | | combination | combination |
| | | | benefit | benefit |
| BMN 673 | DNA_repair | PARP inhib | Synergy | No |
| | | | | combination |
| | | | | benefit |
| BMS 345541 | Signal_inflam | IKK2 inhib | Synergy | No |
| | | | | combination |
| | | | | benefit |
| BMS-708163 | Protein_proc | gSecretase inhib; Regulation; Notch | N/D | N/D |
| BMS-754807 | Signal_grow | IGF-R1 inhib | Synergy | No |
| | | | | combination |
| | | | | benefit |
| BX-912 | Signal_kinase | PDK1 inhib | No | Synergy |
| | | | combination | |
| | | | benefit | |
| C 646 | Regulation | CREB binding protein Inhibitor | Synergy | N/D |
| CAL-101 | Signal_kinase | PI3K inhib | Synergy | No |
| | | | | combination |
| | | | | benefit |
| CAPE | Regulation | NFKB1; NFKB2; REL; RELA; RELB | Synergy | Synergy |
| Carboplatin | DNA_damage | DNA linker | No | No |
| | | | combination | combination |
| | | | benefit | benefit |
| Carfilzomib | Protein_proc | Proteasome | No | No |
| | | | combination | combination |
| | | | benefit | benefit |
| Cerivastatin Sodium | Metabolism | HMGCR | Synergy | Synergy |
| Cerulenin | Metabolism | FASN; HMGCS1; HMGCS2 | No | No |
| | | | combination | combination |
| | | | benefit | benefit |
| CGK 733 | Signal_DNA | ATM; ATR Inhibitor | No | No |
| | repair | | combination | combination |
| | | | benefit | benefit |
| CGP53353 | Signal_kinase | PKCb2 inhib | No | N/D |
| | | | combination | |
| | | | benefit | |
| CHIR 98014 | Signal_apop | GSK3b inhib | No | No |
| | | | combination | combination |
| | | | benefit | benefit |
| Chlorambucil | DNA_damage | DNA alkylator | Synergy | No |
| | | | | combination |
| | | | | benefit |
| Cisplatin | DNA_damage | DNA linker | Synergy | No |
| | | | | combination |
| | | | | benefit |
| Cladribine | DNA_synth | DNApol/PNP inhib | No | No |
| | | | combination | combination |
| | | | benefit | benefit |
| CP-690550 | Signal_kinase | JAK inhib | N/D | N/D |
| CPI-203 | Chromatin | BRD4 | Synergy | Synergy |
| Crizotinib | Signal_grow | ALK inhib | No | No |
| | | | combination | combination |
| | | | benefit | benefit |
| CX-4945 | Signal_cell cycle | CK2 inhib | No | No |
| | | | combination | combination |
| | | | benefit | benefit |
| CZC24832 | Signal_kinase | PI3K inhib | N/D | N/D |
| D609 | Signal | PLC inhib | N/D | N/D |
| Dabrafenib | Signal_kinase | BRAF; CRAF | Synergy | No |
| | | | | combination |
| | | | | benefit |
| Dacarbazine | DNA_damage | DNA alkylator | No | No |
| | | | combination | combination |
| | | | benefit | benefit |
| Dactinomycin | DNA_damage | DNA intercal | No | No |
| | | | combination | combination |
| | | | benefit | benefit |
| DAG Inhibitor II | Signal_kinase | PKC inhib | N/D | N/D |
| Daptomycin | Bacteria_wall | bact nmpC inhib | N/D | N/D |
| Dasatinib | Signal_kinase | BCR-ABL1/Src inhib | No | No |
| | | | combination | combination |
| | | | benefit | benefit |
| Dexamethasone | Regulation | NR3C1 | N/D | N/D |
| Dinaciclib | Signal_cell cycle | CDK inhib | No | No |
| | | | combination | combination |
| | | | benefit | benefit |
| Docetaxel | Cytoskeleton | TUBB1 stab; BCL2 inhib | No | No |
| | | | combination | combination |
| | | | benefit | benefit |
| Dovitinib | Signal_kinase | VEGFR/FGFR/PDGFRB/KIT inhib | No | No |
| | | | combination | combination |
| | | | benefit | benefit |
| Doxorubicin Hcl | DNA_function | TOP2A inhib | No | No |
| | | | combination | combination |
| | | | benefit | benefit |
| Droxinostat | DNA_function | HDAC inhib | No | N/D |
| | | | combination | |
| | | | benefit | |
| Elesclomol | Protein_proc | HSP70 | No | No |
| | | | combination | combination |
| | | | benefit | benefit |
| GSK-LSD1 | Chromatin | LSD1 | Synergy | N/D |
| Epothilone B | Cytoskeleton | TUBB stab | No | No |
| | | | combination | combination |
| | | | benefit | benefit |
| ER 27319 maleate | Signal_kinase | Syk inhib | No | No |
| | | | combination | combination |
| | | | benefit | benefit |
| Erlotinib | Signal_grow | EGFR inhib | No | Synergy |
| Hydrochloride | | | combination | |
| | | | benefit | |
| Etoposide | DNA_function | TOP2A inhib | Synergy | No |
| | | | | combination |
| | | | | benefit |
| ETP-46464 | Signal_DNA | ATR Inhibitor | No | No |
| | repair | | combination | combination |
| | | | benefit | benefit |
| Everolimus | Signal_kinase | mTOR inhib | Synergy | N/D |
| EX 527 | Protein_proc | SIRT1 | N/D | N/D |
| Fingolimod | Sphingosine-1- | | No | No |
| Hydochloride | phosphate | | combination | combination |
| | receptor | | benefit | benefit |
| FK-866 | Metabolism | NMPRTase inhib | No | No |
| | | | combination | combination |
| | | | benefit | benefit |
| Floxuridine | DNA_metab | TYMS inhib | No | No |
| | | | combination | combination |
| | | | benefit | benefit |
| Fluorouracil | DNA_metab | TYMS inhib | No | N/D |
| | | | combination | |
| | | | benefit | |
| Fostamatinib | Signal_kinase | SYK inhib | Synergy | No |
| disodium | | | | combination |
| | | | | benefit |
| Fulvestrant | Hormone_sex | ESR1 inhib | No | N/D |
| | | | combination | |
| | | | benefit | |
| GDC-0449 | Signal_grow | SHH/SMO inhib | N/D | N/D |
| GDC-0879 | Signal_kinase | BRAF inhib | N/D | N/D |
| GDC-0941 | Signal_kinase | PI3K inhib | Synergy | Synergy |
| Gemcitabine | DNA_metab | RRM1/TYMS/POLA inhib | No | No |
| Hydrochloride | | | combination | combination |
| | | | benefit | benefit |
| GF 109203X | Signal_kinase | PKC inhib | No | No |
| | | | combination | combination |
| | | | benefit | benefit |
| Go 6976 | Signal_kinase | PKCa/b1 inhib | Synergy | No |
| | | | | combination |
| | | | | benefit |
| GSK1059615 | Signal_kinase | mTOR; PI3K inhib | No | No |
| | | | combination | combination |
| | | | benefit | benefit |
| GSK1904529A | Signal_grow | IGF-1R inhib | N/D | N/D |
| GSK2656157 | Protein_proc | PERK | Synergy | N/D |
| GSK429286A | Signal_kinase | ROCK inhib | N/D | N/D |
| GSK-J1 | Protein_proc | histone demethylase | N/D | N/D |
| GW2580 | Signal_kinase | c-FMS inhib | N/D | N/D |
| HMN-214 | Signal_cell cycle | PLK inhib | N/D | N/D |
| ICG 001 | Regulation | CREB inhib | No | No |
| | | | combination | combination |
| | | | benefit | benefit |
| IKK 16 | Signal_inflam | IKK1; IKK2 inhibitor | No | No |
| | | | combination | combination |
| | | | benefit | benefit |
| IKK-2 Inhibitor VIII | Signal_inflam | IKK1; IKK2 inhibitor | No | Synergy |
| | | | combination | |
| | | | benefit | |
| Imatinib Mesylate | Signal_kinase | BCR-ABL1/KIT/PDGFRB inhib | N/D | N/D |
| IMD-0354 | Signal_inflam | IKKA inhib | No | No |
| | | | combination | combination |
| | | | benefit | benefit |
| INCB28060 | Signal_grow | c-MET inhib | N/D | N/D |
| IOX1 | Chromatin | JMJD3; JMJD1A; JMJD2A; JMJD2E; JMJD2C; UTX | No | N/D |
| | | | combination | |
| | | | benefit | |
| IPI-145 | Signal_kinase | PI3Kd/g inhib | No | No |
| | | | combination | combination |
| | | | benefit | benefit |
| Irinotecan | DNA_function | TOP1 inhib | Synergy | No |
| Hydrochloride | | | | combination |
| | | | | benefit |
| Ispinesib | Signal_cell cycle | Eg5 inhib | No | No |
| | | | combination | combination |
| | | | benefit | benefit |
| IWP-2 | Signal | WNT inhib | N/D | N/D |
| JIB-04 | Chromatin | JARID1A; JMJD2E; JMJD3; JMJD2A; JMJD2B; JMJD2C; JMJD2D | No | No |
| | | | combination | combination |
| | | | benefit | benefit |
| JNJ 26854165 | Signal_cell cycle | MDM2-p53 inhib | Synergy | No |
| | | | | combination |
| | | | | benefit |
| JNJ-26481585 | DNA_function | HDAC inhib | No | No |
| | | | combination | combination |
| | | | benefit | benefit |
| JNJ-38877605 | Signal_grow | c-Met inhib | N/D | N/D |
| JNK-IN-8 | Signal_kinase | JNK | N/D | N/D |
| Juglone | Protein_proc | PIN4 Pase inhib | No | No |
| | | | combination | combination |
| | | | benefit | benefit |
| KU 0063794 | Signal_kinase | mTOR inhib | Synergy | Synergy |
| KU-55933 | Signal_DNA | ATM Inhibitor | No | N/D |
| | repair | | combination | |
| | | | benefit | |
| KU-60019 | Signal_DNA | ATM Inhibitor | No | N/D |
| | repair | | combination | |
| | | | benefit | |
| Lapatinib | Signal_grow | EGFR/ErbB2 inhib | Synergy | N/D |
| LB42708 | farnesyltransferase | | Synergy | Synergy |
| LDE225 | Signal_grow | SHH; SMO | N/D | N/D |
| LDN 57444 | Protein_proc | UCH-L1 inhib | Synergy | N/D |
| LDN193189 | Signal_grow | ALK2; ALK3 | No | No |
| | | | combination | combination |
| | | | benefit | benefit |
| LEE011 | Signal_cell cycle | CDK4/CDK6 inhib | Synergy | Synergy |
| LIMKi 3 | Signal_kinase | LIMK1; LIMK2 | No | N/D |
| | | | combination | |
| | | | benefit | |
| LY 364947 | Signal_kinase | TGFb | N/D | N/D |
| LY2109761 | Signal_kinase | TGFb | N/D | N/D |
| LY2228820 | Signal_kinase | MAPK14 | No | N/D |
| | | | combination | |
| | | | benefit | |
| LY2603618 | Signal_cell cycle | CHK1 inhib | Synergy | No |
| | | | | combination |
| | | | | benefit |
| Masitinib | Signal_grow | c-Kit; PDGFR; FGFR3; FAK | No | N/D |
| | | | combination | |
| | | | benefit | |
| MC1568 | DNA_function | HDAC inhib | N/D | N/D |
| Mechlorethamine | DNA_damage | DNA alkylator | No | No |
| Hydrochloride | | | combination | combination |
| | | | benefit | benefit |
| Melphalan | DNA_damage | DNA alkylator | Synergy | Synergy |
| Menadione | Signal_cell cycle | CDC25 inhib | Synergy | No |
| | | | | combination |
| | | | | benefit |
| Methylprednisolone | Regulation | GCR ag | N/D | Synergy |
| MGCD-265 | Signal_grow | c-MET; VEGFR; RON; TIE2 | No | N/D |
| | | | combination | |
| | | | benefit | |
| Mifepristone | Regulation | GC-NR3C1 antag | No | No |
| | | | combination | combination |
| | | | benefit | benefit |
| Mitomycin C | DNA_damage | DNA crosslink | Synergy | No |
| | | | | combination |
| | | | | benefit |
| MK 1775 | Signal_cell cycle | Wee1 inhib | No | No |
| | | | combination | combination |
| | | | benefit | benefit |
| MK-2206 | Signal_kinase | AKT inhib | Synergy | Synergy |
| MLN2238 | Protein_PTS | PTS 26S/PSMD2/PSMD1 inhib | No | Synergy |
| | | | combination | |
| | | | benefit | |
| MLN-4924 | Protein_proc | Nedd8 inhibitor | No | No |
| | | | combination | combination |
| | | | benefit | benefit |
| MLN8237 | Signal_cell cycle | Aurora inhib | No | No |
| | | | combination | combination |
| | | | benefit | benefit |
| MLN9708 | Protein_proc | Proteasome | No | No |
| | | | combination | combination |
| | | | benefit | benefit |
| MS 436 | Chromatin | BRD1; BRD2 | Synergy | No |
| | | | | combination |
| | | | | benefit |
| MS-275 | DNA_function | HDAC inhib | Synergy | No |
| | | | | combination |
| | | | | benefit |
| Mycophenolate | DNA_metab | IMPDH inhib | No | No |
| Mofetil | | | combination | combination |
| | | | benefit | benefit |
| Nilotinib | Signal_kinase | BCR-ABL1 inhib | No | No |
| | | | combination | combination |
| | | | benefit | benefit |
| NKH 477 | Signal_gpcr | cAMP activ | No | Synergy |
| | | | combination | |
| | | | benefit | |
| NU 7441 | DNA_repair | DNA-PK | Synergy | No |
| | | | | combination |
| | | | | benefit |
| Nutlin-3 | Signal_cell cycle | MDM2-p53 inhib | Synergy | N/D |
| Olaparib | DNA_repair | PARP inhib | Synergy | No |
| | | | | combination |
| | | | | benefit |
| OTX015 | Chromatin | BRD2; BRD3; BRD4 | Synergy | Synergy |
| Oxaliplatin | DNA_damage | DNA linker | Synergy | N/D |
| PAC 1 | Signal_apop | Procaspase-3 activator | No | No |
| | | | combination | combination |
| | | | benefit | benefit |
| Paclitaxel | Cytoskeleton | TUBB1 stab; BCL2 inhib | No | No |
| | | | combination | combination |
| | | | benefit | benefit |
| Papaverine | Signal_gpcr | PDE6; PDE7; PDE10 | No | Synergy |
| Hydrochloride | | | combination | |
| | | | benefit | |
| Parthenolide | Regulation | NFkB inhib | Synergy | No |
| | | | | combination |
| | | | | benefit |
| Pemetrexed | DNA_metab | TYMS/DHFR/GARFT inhib | No | No |
| | | | combination | combination |
| | | | benefit | benefit |
| Perfosfamide | DNA_damage | DNA alkylator | No | N/D |
| | | | combination | |
| | | | benefit | |
| Perifosine | Signal_kinase | AKT inhib | No | No |
| | | | combination | combination |
| | | | benefit | benefit |
| PF-04217903 | Signal_grow | MET inhib | N/D | N/D |
| PF-04620110 | Metabolism | DGAT1 | N/D | N/D |
| PF-562271 | Signal_kinase | FAK; PYK2 inhib | No | No |
| | | | combination | combination |
| | | | benefit | benefit |
| PFI-3 | Chromatin | polybromol; SMARCA4 | N/D | N/D |
| PHA-793887 | Signal_cell cycle | CDK inhib | No | Synergy |
| | | | combination | |
| | | | benefit | |
| PIK-93 | Signal_kinase | PI3K inhib | No | No |
| | | | combination | combination |
| | | | benefit | benefit |
| PLX-4032 | Signal_kinase | BRAF inhib | No | N/D |
| | | | combination | |
| | | | benefit | |
| Pomalidomide | Signal_inflam | immunosupp | Synergy | N/D |
| RAF265 | Signal_kinase | BRAF inhib | No | No |
| | | | combination | combination |
| | | | benefit | benefit |
| Raloxifene | Hormone_sex | ESR1 antag | Synergy | No |
| Hydrochloride | | | | combination |
| | | | | benefit |
| Resveratrol | Signal_gpcr | PDE4 inhib | No | No |
| | | | combination | combination |
| | | | benefit | benefit |
| RN-486 | Signal_kinase | BTK inhib | N/D | N/D |
| Rosiglitazone | Regulation | PPARg agon | N/D | N/D |
| Ruxolitinib | Signal_kinase | JAK1/JAK2 inhib | No | N/D |
| | | | combination | |
| | | | benefit | |
| SB 415286 | Signal_apop | GSK3b inhib | No | N/D |
| | | | combination | |
| | | | benefit | |
| SB-216763 | Signal_apop | GSK3b inhib | No | N/D |
| | | | combination | |
| | | | benefit | |
| SB-505124 | Signal_grow | ALK4; ALK5 inhib | Synergy | No |
| | | | | combination |
| | | | | benefit |
| SC 514 | Signal_kinase | IKK1/IKK2 inhib | N/D | N/D |
| SC75741 | Signal_inflam | NK-kB | No | No |
| | | | combination | combination |
| | | | benefit | benefit |
| SCH772984 | Signal_kinase | ERK1/2 inhib | Synergy | Synergy |
| SGC-CBP30 | Chromatin | CREBBP/EP300 | No | Synergy |
| | | | combination | |
| | | | benefit | |
| SGI-1776 | Signal_kinase | PIM1 | No | No |
| | | | combination | combination |
| | | | benefit | benefit |
| Sildenafil Citrate | Signal_gpcr | PDE5 | N/D | N/D |
| SMER 3 | Protein_proc | SCF family E3 ubiquitin ligase inhib | Synergy | No |
| | | | | combination |
| | | | | benefit |
| SNS-032 | Signal_cell cycle | CDK inhib | No | No |
| | | | combination | combination |
| | | | benefit | benefit |
| Sorafenib | Signal_kinase | RAF inhib | Synergy | N/D |
| SP 600125 | Signal_kinase | JNK | No | No |
| | | | combination | combination |
| | | | benefit | benefit |
| SRT1720 | Protein_proc | SIRT activ | Synergy | No |
| | | | | combination |
| | | | | benefit |
| Suberoylanilide | DNA_function | HDAC inhib | No | No |
| Hydroxamic Acid | | | combination | combination |
| | | | benefit | benefit |
| Sunitinib Malate | Signal_grow | VEGF2 inhib | No | 2.0 |
| | | | combination | |
| | | | benefit | |
| Tacrolimus (FK-506) | Signal_inflam | FKBP1 inhib | N/D | N/D |
| TAK-715 | Signal_kinase | MAPK14 | No | N/D |
| | | | combination | |
| | | | benefit | |
| Tamoxifen Citrate | Hormone_sex | ESR1 antag | No | No |
| | | | combination | combination |
| | | | benefit | benefit |
| TANSHINONE IIA | Regulation | Nrf2 activ | Synergy | N/D |
| Temozolomide | DNA_damage | DNA alkylator | No | N/D |
| | | | combination | |
| | | | benefit | |
| Temsirolimus | Signal_kinase | mTOR inhib | Synergy | N/D |
| Teniposide | DNA_function | TOP2A inhib | No | No |
| | | | combination | combination |
| | | | benefit | benefit |
| Tenovin-1 | Protein_proc | SIRT1; SIRT2; TP53 | No | No |
| | | | combination | combination |
| | | | benefit | benefit |
| TG101209 | Signal_kinase | JAK2; JAK3; FLT3; RET inhib | No | No |
| | | | combination | combination |
| | | | benefit | benefit |
| Thioguanine | DNA_metab | PPAT/HPRT1/IMPDH1 inhib | No | No |
| | | | combination | combination |
| | | | benefit | benefit |
| Thiostrepton | Regulation | FOXM1 | Synergy | No |
| | | | | combination |
| | | | | benefit |
| Thiotepa | DNA_damage | DNA alkylation | Synergy | No |
| | | | | combination |
| | | | | benefit |
| Tipifarnib | farnesyltransferase | | Synergy | No |
| | | | | combination |
| | | | | benefit |
| Topotecan | DNA_function | TOP1 inhib | Synergy | No |
| Hydrochloride | | | | combination |
| | | | | benefit |
| Trequinsin | Signal_gpcr | PDE2; PDE3; PDE4 | No | N/D |
| Hydrochloride | | | combination | |
| | | | benefit | |
| Tretinoin | Regulation | RAR bind | Synergy | Synergy |
| Triciribine | Signal_kinase | AKT inhib | Synergy | N/D |
| Tubastatin A | Protein_proc | HDAC6 | No | No |
| hydrochloride | | | combination | combination |
| | | | benefit | benefit |
| TW-37 | Signal_apop | BCL-2 antag | No | No |
| | | | combination | combination |
| | | | benefit | benefit |
| UNC 0646 | Chromatin | G9a and GLP histone lysine methyltransferases | Synergy | Synergy |
| UNC1215 | Chromatin | L3MBTL3 | N/D | N/D |
| UNC1999 | Chromatin | EZH1; EZH2 | No | N/D |
| | | | combination | |
| | | | benefit | |
| VE-821 | Signal_DNA | ATR Inhibitor | Synergy | Synergy |
| | repair | | | |
| Vinblastine Sulfate | Cytoskeleton | TUBB2 destab | No | No |
| | | | combination | combination |
| | | | benefit | benefit |
| WIKI4 | Signal | WNT inhib | N/D | N/D |
| XAV-939 | Regulation | TNKS1; TNKS2 | No | N/D |
| | | | combination | |
| | | | benefit | |
| XL147 | Signal_kinase | PI3K inhib | Synergy | N/D |
| XL184 | Signal_kinase | VEGFR2; c-MET; RET; KIT; Flt1/3/4; Tie2; AXL | No | No |
| | | | combination | combination |
| | | | benefit | benefit |
| YM155 | Signal_apop | Survivin inhib | No | No |
| | | | combination | combination |
| | | | benefit | benefit |

### EXAMPLE 7

### Additional Combination Studies

### Methods

For the primary screen, MOLM-13 cells, OCI-AML-4 cells, ML-2 cells, THP-1 cells, RS4-11 cells, or SKM-1 cells were pre-treated with 300 nM of EPZ-5676 (i.e., pinometostat or Compound A2) or DMSO in T175 flasks for a 4-day pre-treatment time. Cells were split using EPZ-5676 or DMSO containing growth media and further incubated for an additional 3-day pre-treatment time. Cells were finally seeded in growth media containing EPZ-5676 or DMSO in 384-well plates at 500 cell/well density. Cells were then equilibrated in incubators for 24 hours before treatment with a second compound. Treated assay plates were incubated with a second compound for 72 hours. After this time, plates were developed for endpoint analysis using ATPLite to measure ATP content, which is used as an indicator of cell viability.

For the retest, MOLM-13, ML-2, THP-1, RS4-11, SKM-1 and OCI-AML-4 cells were pretreated with EPZ-5676 (i.e., Compound A2; 150 nM for MOLM-13 and 300 nM for ML-2, THP-1, RS4-11, SKM1 and OCI-AML-4) or DMSO in T175 flasks for a 4-day pretreatment time. Cells were split using EPZ-5676 or DMSO containing growth media and further incubated for an additional 3-day pre-treatment time. Cells were finally seeded in growth media containing EPZ-5676 or DMSO in 96-well plates. Cells were then treated with a second compound for 72 hours. After this time, plates were developed for endpoint analysis using Cell Titer Glo to measure ATP content, which is used as an indicator of cell viability.

A combination of EPZ-5676 and a second compound was considered synergistic if the GI100 (Growth inhibition 100) value of the second compound decreased by 2-fold or more when EPZ-5676 was added as compared to the DMSO control.

The results of these combinations are summarized in Tables 7 and 8 below. “N/D” means the GI100 of the second compound could not be determined for both conditions and “-” means not tested.

TABLE 7

| | | MOLM-13 (MLL-AF9) | | OCI-AML-4 (MLL-ENL) | | SKM-1 (non-MLLr) | |
|---|---|---|---|---|---|---|---|
| Drug | Target | Primary screen | Retest | Primary screen | Retest | Primary screen | Retest |
| Trametinib | MEK | Synergy | Synergy | Synergy | Synergy | Synergy | Synergy |
| Selumetinib (AZD6244) | | Synergy | Synergy | Synergy | Synergy | Synergy | Synergy |
| Binimetinib (MEK-162) | | — | Synergy | — | Synergy | — | Synergy |
| SCH772984 | ERK | Synergy | Synergy | Synergy | Synergy | Synergy | Synergy |
| GDC0994 | | — | No combination | — | Synergy | — | Synergy |
| | | | benefit | | | | |
| Ulixertinib | | — | Synergy | — | Synergy | — | Synergy |
| RAF265 | RAF | Synergy | — | No combination | — | N/D | — |
| | | | | benefit | | | |
| Sorafenib | | Synergy | Synergy | No combination | No combination | N/D | N/D |
| | | | | benefit | benefit | | |
| GDC-0879 | | N/D | — | N/D | — | N/D | — |
| PLX-4032 | | No combination | — | N/D | — | N/D | — |
| | | benefit | | | | | |
| Dabrafenib | | N/D | — | N/D | — | N/D | — |
| EPZ-5676 10-day (Retest) or 11-day | | 0.15 | 0.075 | >3 | >3 | >3 | >3 |
| (Primary screen) IC₅₀ [uM] | | | | | | | |

TABLE 8

| | Ras | | | | OCI-AML-4 | | | | | |
|---|---|---|---|---|---|---|---|---|---|---|
| | Mutation | MOLM-13 (MLL-AF9) | | | (MLL-ENL) | | | THP-1 (MLL-AF9) | | |
| | Status | None known | | | NRAS Q61R (het) | | | NRAS G12D | | |
| Drug | Target | GI₅₀ | GI₁₀₀ | Result | GI₅₀ | GI₁₀₀ | Result | GI₅₀ | GI₁₀₀ | Result |
| Trametinib | MEK | 0.01 | 0.03 | Synergy | 0.002 | 0.009 | Synergy | 0.002 | N/D | No |
| | | | | | | | | | | combination |
| | | | | | | | | | | benefit |
| Selumetinib | | 1.25 | 7.28 | Synergy | 0.116 | 3.169 | Synergy | 0.16 | >10 | No |
| (AZD6244) | | | | | | | | | | combination |
| | | | | | | | | | | benefit |
| Binimetinib (MEK- | | 0.72 | 3.96 | Synergy | 0.059 | 1.504 | Synergy | 0.09 | >10 | No |
| 162) | | | | | | | | | | combination |
| | | | | | | | | | | benefit |
| SCH772984 | ERK | 0.21 | 0.53 | Synergy | 0.070 | 0.134, | Synergy | 0.06 | >10 | No |
| | | | | | | 0.087 | | | | combination |
| | | | | | | | | | | benefit |
| GDC0994 | | 3.08 | 4.26 | No | 0.904 | 3.068 | Synergy | 2.85 | >10 | No |
| | | | | combination | | | | | | combination |
| | | | | benefit | | | | | | benefit |
| Ulixertinib | | 1.67 | 3.96 | Synergy | 2.105 | 7.128, | Synergy | 0.81 | 3.28, | No |
| | | | | | | >10 | | | >10 | combination |
| | | | | | | | | | | benefit |

| | | | | |
|---|---|---|---|---|
| EPZ-5676 10-day | DOT1L | 0.075 | >3 | >3 |
| IC50 μM | | | | |

| | Ras | | | | | | | | | |
|---|---|---|---|---|---|---|---|---|---|---|
| | Mutation | ML-2 (MLL-AF6) | | | RS4-11 (MLL-AF4) | | | SKM-1 (non-MLLr) | | |
| | Status | KRAS A146T (het) | | | None known | | | KRAS K117N (homo) | | |
| Drug | Target | GI₅₀ | GI₁₀₀ | Result | GI₅₀ | GI₁₀₀ | Result | GI₅₀ | GI₁₀₀ | Result |
| Trametinib | MEK | 0.001 | 0.001 | Synergy | >10 | >10 | N/D | 0.001 | 0.004 | Synergy |
| Selumetinib | | 0.04 | 0.93 | No | >10 | >10 | N/D | 0.02 | 0.12 | Synergy |
| (AZD6244) | | | | combination | | | | | | |
| | | | | benefit | | | | | | |
| Binimetinib | | 0.03 | 0.50 | Synergy | >10 | >10 | N/D | 0.01 | 0.07 | Synergy |
| (MEK-162) | | | | | | | | | | |
| SCH772984 | ERK | 0.04 | 0.26 | Synergy | 7.243 | >10 | N/D | 0.02 | 0.086 | Synergy |
| GDC0994 | | 0.59 | 2.55 | No | 1.299 | 2.505 | No | 0.27 | 1.84 | Synergy |
| | | | | combination | | | combination | | | |
| | | | | benefit | | | benefit | | | |
| Ulixertinib | | 0.63 | 2.711 | Synergy | >10 | >10 | N/D | 0.44 | 1.500 | Synergy |

| | | | | |
|---|---|---|---|---|
| EPZ-5676 10-day | DOT1L | >3 | >3 | >3 |
| IC50 μM | | | | |

Growth Inhibition (GI) as a measure of cell viability: The cell viability of vehicle was measured at the time of dosing the second agent (T₀) and after seventy-two hours (T₇₂). A GI reading of 0% represents no growth inhibition—cells treated with test compound as compared to T₇₂ vehicle signals were measured. A GI 100% represents complete growth inhibition—cells treated with test compound as compared to T₀ vehicle signals were measured. Cell numbers have not increased during the treatment period in wells with GI 100% and may suggest a cytostatic effect for compounds reaching a plateau at this effect level. A GI 200% represents complete death of all cells in the culture well. Compounds reaching an activity plateau of GI 200% are considered cytotoxic. GI is calculated by applying the following test and equation:

$$if T < {V}_{0} \text{:} 100 * ( 1 - \frac{T - {V}_{0}}{{V}_{0}} )$$

$$if T \geq {V}_{0} \text{:} 100 * ( 1 - \frac{T - {V}_{0}}{V - {V}_{0}} ) ,$$

where T is the signal measure for a test article, V is the vehicle-treated control measure, and Vₒ is the vehicle control measure at time zero.

As shown in Tables 7 and 8, among the most compelling findings was synergistic activity of pinometostat with several modulators of the MAP kinase pathway (e.g., trametinib, an approved MEK inhibitor) in multiple MLL-r cell lines. Study of dosing schedule of the combination of pinometostat with trametinib revealed that all schedules, no matter the order of compound addition, demonstrated combination benefit. Pretreatment with the DOT1L inhibitor, however, elicited dramatic cell killing at physiologically achievable concentrations.

When compared to monotherapy, the results indicate that combinatorial treatment of pinometostat with trametinib boosts the inhibitory effect on cell lines sensitive and resistant to DOT1L inhibition. Taken together, these findings imply that suppression of DOT1L activity prior to MEK inhibition may have advantages over monotherapy of either agent.

### EXAMPLE 8

### DOT1L Inhibitor Compound A2 Displays Synergistic Antiproliferative Activity in Combination with PPAR Antagonist in MLL-Rearranged Leukemia Cells

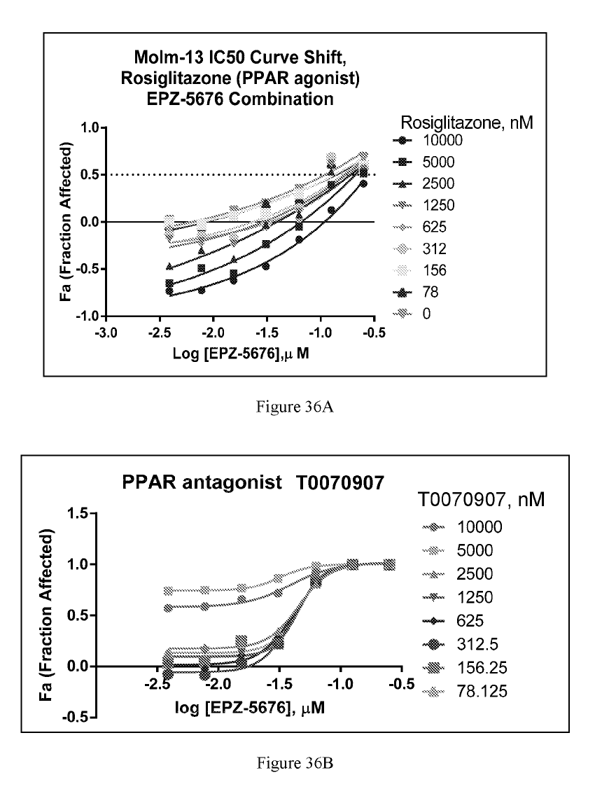

Compound A2 and a second therapeutic agent (Rosiglitazone or T0070907) were administered to the MOLM-13 cells according to the methods disclosed in previous examples, such as Example 3, co-treatment model. Results from the combinational administration were listed in the table below and illustrated in FIGS. 36A and 36B.

| | Rosiglitazone | T0070907 PPAR |
|---|---|---|
| | PPAR agonist | antagonist |
| EPZ-5676 | Antagonistic effect - | Synergy |
| | Increased | |
| | proliferation | |

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A combination comprising:
i) a compound selected from Compound A2, Compound D16, and pharmaceutically acceptable salts thereof; and
ii) one or more therapeutic agents.

2. The combination of claim 1, comprising Compound A2 or a pharmaceutically acceptable salt thereof, and one or more therapeutic agents.

3. The combination of claim 1, comprising Compound D16 or pharmaceutically acceptable salt thereof, and one or more therapeutic agents.

4. The combination of claim 1, wherein the one or more therapeutic agents are anti-cancer agents.

5. The combination of claim 1, wherein the one or more therapeutic agents are selected from Ara-C, Daunorubicin, Azacitidine, Decitabine, Panobinostat, Vidaza, Mitoxantrone, Methotrexate, Mafosfamide, Prednisolone, Vincristine, Lenalidomide, Hydroxyurea, Menin-MLL inhibitor MI-2, JQ1, IBET151, Vorinostat, Quizartinib, Midostaurin, Tranylcypromine, LSD1 inhibitor II, Navitoclax, Velcade, SRT-1720, Furazolidone, Fludarabine, Mercaptopurine, Obatoclax, ABT-199, Trametinib, Clofarabine, Ibrutinib, Palbociclib, AZ20, MK2206, BEZ235, T0070907, Romidepsin, Tipifarnib, Volasertib, Compound E10, 10-Hydroxycamptothecin, ABT-737, Alitretinoin, AT7867, Auranofin, AZD 8055, AZD6244, Baricitinib, BEP800, Bexarotene, BIX01294, Bleomycin Sulfate, BMN 673, BMS 345541, BMS-754807, BX-912, C 646, CAL-101, CAPE, Cerivastatin Sodium, Chlorambucil, Cisplatin, CPI-203, Dabrafenib, GSK-LSD1, Erlotinib Hydrochloride, Etoposide, Everolimus, Fostamatinib disodium, GDC-0941, Go 6976, GSK2656157, IKK-2 Inhibitor VIII, Irinotecan Hydrochloride, JNJ 26854165, KU 0063794, Lapatinib, LB42708, LDN 57444, LEE011, LY2603618, Melphalan, Menadione, Methylprednisolone, Mitomycin C, MK-2206, MLN2238, MS 436, MS-275, NKH 477, NU 7441, Nutlin-3, Olaparib, OTX015, Oxaliplatin, Papaverine Hydrochloride, Parthenolide, PHA-793887, Pomalidomide, Raloxifene Hydrochloride, SB-505124, SCH772984, SGC-CBP30, SMER 3, Sorafenib, SRT1720, TANSHINONE IIA, Temsirolimus, Thiostrepton, Thiotepa, Topotecan Hydrochloride, Tretinoin, Triciribine, UNC 0646, VE-821, XL147, and analogs, derivatives, or combinations thereof.

6. The combination of claim 1, wherein the one or more therapeutic agents are selected from Ara-C, Daunorubicin, Decitabine, Vidaza, Mitoxantrone, JQ1, IBET151, Panobinostat, Vorinostat, Quizartinib, Midostaurin, Tranylcypromine, LSD1 inhibitor II, Navitoclax, and analogs, derivatives, or combinations thereof.

7. The combination of claim 1, wherein the therapeutic agent is Ara-C, Daunorubicin, Vidaza, a PPAR antagonist or an analog or derivative thereof.

8. The combination of claim 1, wherein the therapeutic agent is Ara-C, Daunorubicin, or an analog or derivative thereof.

9. The combination of claim 1, wherein the therapeutic agent is Vidaza or an analog or derivative thereof.

10. The combination of claim 1, wherein the therapeutic agent is a MEK1 inhibitor, a MEK2 inhibitor, an ERK inhibitor, a RAF inhibitor or a RAS inhibitor.

11. The combination of claim 1, wherein the therapeutic agent is trametinib or an analog or derivative thereof.

12. A pharmaceutical composition comprising a therapeutically effective amount of the combination of claim 1 and a pharmaceutically acceptable carrier.

13. A method of treating or alleviating a symptom of a disease comprising administering to a subject in need thereof a therapeutically effective amount of the combination of claim 1.

14-16. (canceled)

17. A method of treating or alleviating a symptom of cancer comprising administering to a subject in need thereof a therapeutically effective dose of a compound selected from Compound A2, Compound D16, and pharmaceutically acceptable salts thereof, and one or more therapeutic agents, wherein the compound and the one or more therapeutic agents are administered simultaneously or sequentially.

18. (canceled)

19. A method of treating or alleviating a symptom of cancer comprising administering to a subject in need thereof a therapeutically effective dose of a compound selected from Compound A2, Compound D16, and pharmaceutically acceptable salts thereof, prior to administering a therapeutically effective dose of the combination of claim 1.

20-42. (canceled)

43. A method of inhibiting cancer cell proliferation comprising contacting a cancer cell with a compound selected from Compound A2, Compound D16, and pharmaceutically acceptable salts thereof, and one or more therapeutic agents, wherein the compound and the therapeutic agents are delivered simultaneously or sequentially.

44. (canceled)

45. A method of inhibiting cancer cell proliferation comprising administering to a subject in need thereof a therapeutically effective dose of a compound selected from Compound A2, Compound D16, and pharmaceutically acceptable salts thereof, prior to administering a therapeutically effective dose of the combination of claim 1.

46-52. (canceled)

53. A method of treating or alleviating a symptom of a disease comprising administering to a subject in need thereof a therapeutically effective amount of a compound selected from Compound A2, Compound D16, and pharmaceutically acceptable salts thereof, wherein the therapeutically effective amount is an amount sufficient to sensitize the subject to subsequent treatment with a therapeutic agent.

54-69. (canceled)

70. A method of treating or alleviating a symptom of cancer comprising administering to a subject in need thereof a therapeutically effective dose of one or more therapeutic agents prior to administering a therapeutically effective dose of the combination of claim 1.

71-72. (canceled)

73. A method of treating or alleviating a symptom of a disease comprising administering to a subject in need thereof a therapeutically effective amount of one or more therapeutic agents, wherein the therapeutically effective amount is an amount sufficient to sensitize the subject to subsequent treatment with a compound selected from Compound A2, Compound D16, and pharmaceutically acceptable salts thereof, or combination of claim 1.

74-95. (canceled)
